(19) United States
(12) Patent Application Publication
(10) Pub. No.: US 2024/0132577 A1
(43) Pub. Date: Apr. 25, 2024

(54) SINGLE DOMAIN ANTIBODIES THAT NEUTRALIZE SARS-CoV-2

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Rafael Cristian CASELLAS, Kensington, MD (US); Jianliang XU, North Potomac, MD (US)

(21) Appl. No.: 18/546,933

(22) PCT Filed: Feb. 18, 2022
(86) PCT No.: PCT/US2022/016986

§ 371 Date: Aug. 16, 2023

Related U.S. Application Data
(60) Provisional application No. 63/151,530, filed on Feb. 19, 2021.

(51) Int. Cl. C07K 16/10; G01N 33/569

(52) U.S. Cl. CPC C07K 16/1003; G01N 33/56983; C07K 2317/22; C07K 2317/569; C07K 2317/76; C07K 2317/92; G01N 2333/165; G01N 2469/10

(57) ABSTRACT

Single-domain antibodies against SARS-CoV-2 are provided. The single-domain antibodies have been shown to have neutralizing activity against SARS-CoV-2 and can be used as a diagnostic and/or therapeutic in patients with coronavirus infection, such as COVID-19; and in diseases and disorders related to, or resulting from, coronavirus infection.

### GOVERNMENT INTERESTS—NIH GRANT INFO

This invention was made partly using funds from the following United States Federal agencies: The Intramural Programs of the Division of Intramural Research, NIAMS, NIH. The US Federal Government has certain rights to this invention.

### REFERENCE TO SEQUENCE LISTING SUBMITTED ELECTRONICALLY

Pursuant to the EFS-Web legal framework and 37 CFR §§ 1.821-825 (see MPEP § 2442.03(a)), a Sequence Listing in the form of an ASCII-compliant text file (entitled “Sequence_Listing_3000093-005977_ST25.txt” created on 17 Feb. 2022, and 132,895 bytes in size) is submitted concurrently with the instant application, and the entire contents of the Sequence Listing are incorporated herein by reference.

### BACKGROUND

### 1. Field

The present disclosure relates to single domain antibodies against SARS-CoV-2. The single domain antibodies block SARS-CoV-2 infection, and can be used as a therapeutic, prophylactic, and/or diagnostic in patients.

### 2. Description of Related Art

Viral infections are a continued problem for public health. In the 20th and 21st centuries, pandemics have been caused by novel viruses.

Coronavirus disease (COVID-19) is an infectious disease caused by a newly discovered coronavirus, Severe Acute Respiratory Syndrome CoronaVirus 2 (SARS-CoV-2). SARS-CoV-2 was discovered in Wuhan Viral Pneumonia cases in late 2019, and was named by the World Health Organization on Jan. 12, 2020. It belongs to the beta genera of the Coronaviridae family identified in 2003, together with SARS coronavirus (SARS CoV), identified in 2003 and MERS coronavirus (MERS CoV) identified in 2012. The SARS-CoV-2 genome shares about 70% sequence identity with the SARS CoV virus and about 40% sequence similarity with the MERS CoV virus. WHO website (2020).

Most people infected with the SARS-CoV-2 virus will experience mild to moderate respiratory illness and recover without requiring special treatment. Older patients, e.g., over 60 years of age, and those with underlying medical problems such as cardiovascular disease, diabetes, chronic respiratory disease, and cancer, are more likely to develop serious illness. Centers for Disease Control website (2020). In susceptible populations, severe acute respiratory syndrome coronavirus 2 (SARS-CoV-2) may cause fatal human respiratory disease. Patients with COVID-19 often display the characteristics of acute lung injury (ALI), including diffuse alveolar damage (DAD), epithelial necrosis, and fibrin and hyaline deposition. Many patients who die of COVID-19 develop acute respiratory distress syndrome (ARDS), a severe form of acute lung injury. Li & Ma Critical Care (2020) 24:198. Outbreaks of severe acute respiratory infections of emerging viruses, including Middle Eastern respiratory syndrome CoVs (MERS-CoV) and the novel SARS-CoV-2 show a need in the art for effective agents for the treatment and/or prevention of coronavirus infections.

Similar to other coronaviruses, the spike glycoprotein (S) homotrimer on SARS-CoV-2 plays a pivotal role in receptor binding and viral entry. The spike glycoprotein is segregated into two functional subunits, termed S1 and S2. The S1 subunit is responsible for the binding of host cell receptor via the interaction between its C-terminal receptor-binding domain (RBD) and human angiotensin converting enzyme 2 (ACE2). S2 subunit plays an important role in fusion of the viral and cellular membranes.

Several drugs and vaccines are under development for the prevention and treatment of COVID-19. Biologic drugs neutralizing SARS-CoV-2-RBD/ACE2 interaction, especially, are of interest. There are at least 9 SARS-CoV-2-RBD antibodies or combination of antibodies being evaluated in clinical trials, with REGN-COV2 being the most advanced (NCT04452318, Phase 3).

There exists a need in the art for anti-COVID-19 therapeutics.

### BRIEF SUMMARY

The present disclosure provides single-domain antibodies (including but not limited to antigen-binding molecules comprising, or alternatively consisting of, antibody fragments or variants thereof) that immunospecifically bind to SARS-CoV-2, compositions, and methods of use thereof. For example, the disclosure provides single-domain antibodies (including antigen-binding molecules comprising, or alternatively consisting of, antibody fragments or variants thereof) that immunospecifically bind to the receptor binding domain (RBD) of SARS-CoV-2. The present invention also provides methods and compositions for detecting, diagnosing, or prognosing diseases or disorders associated with SARS-CoV-2 in an animal, preferably a mammal, and most preferably a human, comprising, or alternatively consisting of, use of single-domain antibodies (including, but not limited to antigen-binding molecules comprising, or alternatively consisting of, antibody fragments or variants thereof) that immunospecifically bind to SARS-CoV-2. Diseases and disorders which can be detected, diagnosed, or prognosed with the disclosed single-chain antibodies (including, but not limited to antigen-binding molecules comprising, or alternatively consisting of, antibody fragments or variants thereof) include, but are not limited to, COVID-19. The present disclosure further provides methods and compositions for preventing, treating or ameliorating diseases or disorders associated with SARS-CoV-2 in an animal, preferably a mammal, and most preferably a human, comprising, or alternatively consisting of, administering to said animal an effective amount of one or more single-domain antibodies (including antigen-binding molecules comprising, or alternatively consisting of, antibody fragments or variants thereof) that immunospecifically bind to SARS-CoV-2. Diseases and disorders which can be prevented, treated or ameliorated by administering an effective amount of an antibody described herein include, but are not limited to, COVID-19.

In an embodiment, the single-domain antibodies described herein immunospecifically bind to SARS-CoV-2, in particular, to the RBD of SARS-CoV-2. The single-domain antibodies described herein may be an amino acid sequence that comprises an immunoglobulin fold or may be an amino acid sequence that, under suitable conditions (e.g., physiological conditions), is capable of forming an immunoglobulin fold (e.g., by folding). Preferably, when properly folded so as to form an immunoglobulin fold, such an amino acid sequence is capable of immunospecifically binding to SARS-CoV-2; and more preferably capable of immunospecifically binding to the RBD of SARS-CoV-2.

In an embodiment, the antigen-binding molecules described herein comprise, or alternatively consist of, fragments or variants of these single-domain antibodies (e.g., including an amino acid sequence of any one of those described herein), that immunospecifically binds to SARS-CoV-2, preferably the RBD of SARS-CoV-2. In an embodiment, nucleic acid molecules that encode the single-domain antibodies described herein, vectors and host cells comprising the same, and/or antigen-binding molecules are also provided.

In one embodiment, single-domain antibodies described herein comprise amino acid sequences that consist essentially of four framework regions (FR1, FR2, FR3, FR4) and three complementarity determining regions (CDR1, CDR2, CDR3); or any suitable fragment of such an amino acid sequence (which will usually comprise at least some of the amino acid residues that form at least one of the CDRs, as further described herein).

In an embodiment, the single-domain antibodies described herein may be an immunoglobulin sequence or a suitable fragment thereof, and, in particular, may be an immunoglobulin variable domain sequence, immunoglobulin single variable domain sequences or a suitable fragment thereof, such as light chain variable domain sequence (e.g., a VL domain sequence) or a suitable fragment thereof; or a heavy chain variable domain sequence (e.g., a VH domain sequence) or a suitable fragment thereof. When the single-domain antibody described herein is a heavy chain variable domain sequence, it may be a heavy chain variable domain sequence that is derived from a conventional four-chain antibody (e.g., a VH domain sequence that is derived from a human antibody) or be a VHH sequence (as described herein) that is derived from a heavy chain antibody (as described herein).

However, it should be noted that the single-domain antibodies described herein are not limited as to the origin of the amino acid sequences described herein (or of the nucleotide sequences described herein used to express them), nor as to the way that the amino acid sequences or nucleotide sequences described herein are (or have been) generated or obtained. Thus, the amino acid sequences described herein may be naturally-occurring amino acid sequences (from any suitable species) or synthetic or semi-synthetic amino acid sequences. In a specific, but non-limiting embodiment, the amino acid sequences described herein are naturally-occurring immunoglobulin sequences (from any suitable species) or synthetic or semi-synthetic immunoglobulin sequences, including, but not limited to, “humanized” immunoglobulin sequences (including but not limited to partially or fully humanized mouse or rabbit immunoglobulin sequences, and in particular partially or fully humanized VHH sequences), “camelized” immunoglobulin sequences, as well as immunoglobulin sequences that have been obtained by techniques including but not limited to affinity maturation (e.g., starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, the nucleotide sequences described herein may be naturally-occurring nucleotide sequences or synthetic or semi-synthetic sequences, and may, for example, be sequences that are isolated by PCR from a suitable naturally-occurring template (e.g., DNA or RNA isolated from a cell), nucleotide sequences that have been isolated from a library (e.g., an expression library), nucleotide sequences that have been prepared by introducing mutations into a naturally-occurring nucleotide sequence (using any suitable technique such as mismatch PCR), nucleotide sequences that have been prepared by PCR using overlapping primers, or nucleotide sequences that have been prepared using any known techniques for DNA synthesis.

In an embodiment, the single-domain antibodies described herein comprise, or alternatively consist of, an immunoglobulin single variable domain sequence such as a domain antibody (or an amino acid sequence that is suitable for use as a domain antibody), a single-domain antibody (or an amino acid sequence that is suitable for use as a single domain antibody), a “dAb” (or an amino acid sequence that is suitable for use as a dAb) or a VHH sequence; other single variable domains, or any suitable fragment of any one thereof. For a general description of (single) domain antibodies, reference is made to EP 0 368 684. For the term “dAb's”, reference is for example made to Ward et al. (Nature 1989 Oct. 12; 341(6242): 544-6), Holt et al., Trends Biotechnol., 2003, 21(11):484-490; as well as to, for example, WO 2006/030220 and WO 2006/003388. It should also be noted that, although less preferred in the context described herein because they are not of mammalian origin, single-domain antibodies or single variable domains can be derived from certain species of shark (e.g., “IgNAR domains”, see for example WO 2005/18629).

In an embodiment, the single-domain antibodies described herein comprise, or alternatively consist of, amino acid sequences with the structure: FR1-CDR1-FR2-CDR2-FR3-CDR3-FR4, in which FR1 to FR4 refer to framework regions 1 to 4, respectively, and CDR1 to CDR3 refer to complementarity determining regions 1 to 3, respectively.

In an embodiment, the single-domain antibodies described herein comprise, or alternatively consist of, an amino acid sequence with the structure: FR1-CDR1-FR2-CDR2-FR3-CDR3-FR4, in which the amino acid sequence has at least 80% identity to at least one of the amino acid sequences of SEQ ID NOS: 1 to 15.

Antigen-binding molecules comprising, or alternatively consisting of, fragments or variants of these single-domain antibodies (e.g., including one or more CDRs having an amino acid sequence of any one of those described herein) that immunospecifically bind SARS-CoV-2 are also provided by the disclosure, as are nucleic acid molecules that encode these antibodies, vectors and host cells comprising the same, and/or antigen-binding molecules.

In an embodiment, the amino acid sequence described herein may be an amino acid sequence that comprises at least one amino acid sequence that is chosen from the group consisting of the CDR1 sequences, CDR2 sequences and CDR3 sequences described herein (or any suitable combination thereof). In particular, an amino acid sequence described herein may be an amino acid sequence that comprises at least one antigen binding site, wherein said antigen binding site comprises at least one amino acid sequence that is chosen from the group consisting of the CDR1 sequences, CDR2 sequences and CDR3 sequences described herein (or any suitable combination thereof).

In an embodiment, the amino acid sequence described herein may be any amino acid sequence that comprises at least one stretch of amino acid residues, in which the stretch of amino acid residues has an amino acid sequence that corresponds to the sequence of at least one of the CDR sequences described herein. Such an amino acid sequence may or may not comprise an immunoglobulin fold. In a non-limiting example, such an amino acid sequence may be a suitable fragment of an immunoglobulin sequence that comprises at least one CDR sequence, but that is not large enough to form a (complete) immunoglobulin fold (see, e.g., “Expedite fragments” described in WO 2003/050531 or WO 2009/127691). Alternatively, such an amino acid sequence may be a suitable “protein scaffold” that comprises at least one stretch of amino acid residues that corresponds to a CDR sequence (e.g., as part of its antigen binding site). Suitable scaffolds for presenting amino acid sequences will be clear to the skilled person, for example, binding scaffolds based on or derived from immunoglobulins (e.g., other than the immunoglobulin sequences described herein), protein scaffolds derived from protein A domains (e.g., Affibodies®), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9(8):619-32).

Any amino acid sequence described herein comprising one or more of the CDR sequences described herein preferably immunospecifically binds to SARS-CoV-2, more preferably to the RBD of SARS-CoV-2.

In an embodiment, the amino acid sequences according to the present disclosure may be any amino acid sequence that comprises at least one antigen binding site, wherein said antigen binding site comprises at least two amino acid sequences that are selected from the group consisting of the CDR1 sequences described herein, the CDR2 sequences described herein, and the CDR3 sequences described herein, such that (i) when the first amino acid sequence is selected from the CDR1 sequences described herein, the second amino acid sequence is selected from the CDR2 sequences described herein or the CDR3 sequences described herein; (ii) when the first amino acid sequence is selected from the CDR2 sequences described herein, the second amino acid sequence is selected from the CDR1 sequences described herein or the CDR3 sequences described herein; or (iii) when the first amino acid sequence is selected from the CDR3 sequences described herein, the second amino acid sequence is selected from the CDR1 sequences described herein or the CDR3 sequences described herein.

In an embodiment, the amino acid sequences described herein may be amino acid sequences that comprise at least one antigen binding site, wherein said antigen binding site comprises at least three amino acid sequences that are selected from the group consisting of the CDR1 sequences described herein, the CDR2 sequences described herein and the CDR3 sequences described herein, such that the first amino acid sequence is selected from the CDR1 sequences described herein, the second amino acid sequence is selected from the CDR2 sequences described herein, and the third amino acid sequence is selected from the CDR3 sequences described herein.

Combinations of CDR1, CDR2 and CDR3 sequences are described herein. The amino acid sequence comprising CDR1, CDR2, and CDR3 is preferably an immunoglobulin sequence (as further described herein), but it may for example also be any other amino acid sequence that comprises a suitable scaffold for presenting said CDR sequences.

In one embodiment, single-domain antibodies (including molecules comprising, or alternatively consisting of, antibody fragments or variants thereof) may immunospecifically bind to a polypeptide or a polypeptide fragment of SARS-CoV-2, said antibodies comprising, or alternatively consisting of, a polypeptide having the amino acid sequence of any one, two, or three of the CDRs (e.g., CDR1, CDR2, or CDR3) of a VHH domain having an amino acid sequence of any one of SEQ ID NOS: 1 to 15. In one embodiment, single-domain antibodies described herein comprise a polypeptide having the amino acid sequence of any one of the CDR1 s of a VHH domain having an amino acid sequence of any one of SEQ ID NOS: 1 to 15. In an embodiment, single-domain antibodies described herein comprise a polypeptide having the amino acid sequence of any one of the CDR2s of a VHH domain having an amino acid sequence of any one of SEQ ID NOS: 1 to 15. In an embodiment, single-domain antibodies described herein comprise a polypeptide having the amino acid sequence of any one of the CDR3s of a VHH domain having an amino acid sequence of any one of SEQ ID NOS: 1 to 15. Molecules comprising, or alternatively consisting of, fragments or variants of the VHH domains of any one of SEQ ID NOS: 1 to 15, (e.g., CDRs) that immunospecifically bind to SARS-CoV-2, preferably to the RBD of SARS-CoV-2, are also provided by the disclosure, as are nucleic acid molecules that encode these antibodies, and/or molecules.

In an embodiment, single-domain antibodies described herein (including molecules comprising, or alternatively consisting of, antibody fragments or variants thereof) immunospecifically bind to a polypeptide or polypeptide fragment of SARS-CoV-2, and comprise, or alternatively consist of, a polypeptide having the amino acid sequence of any one of the CDR1 s of the VHH domains of any one of SEQ ID NOS: 1 to 15, any one of the CDR2s of the VHH domains of any one of SEQ ID NOS: 1 to 15, and/or any one of the CDR3s of the VHH domains of any one of SEQ ID NOS: 1 to 15. In an embodiment, single-domain antibodies described herein comprise amino acid sequences of CDR1, CDR2, and CDR3 of the VHH domain of any one of SEQ ID NOS: 1 to 15.

In an embodiment, the single-domain antibodies described herein immunospecifically binds to SARS-CoV-2 and comprise one or more amino acid sequences selected from the group consisting of:
a) an amino acid sequence of any one of SEQ ID NOS: 27 to 40;
b) an amino acid sequence having at least 80% identity to at least one of the amino acid sequences of SEQ ID NOS: 27 to 40;
c) an amino acid sequence having 3, 2, or 1 amino acid differences with at least one of the amino acid sequences of SEQ ID NOS: 27 to 40;
d) an amino acid sequence of any one of SEQ ID NOS: 49 to 62;
e) an amino acid sequence having at least 80% identity to at least one of the amino acid sequences of SEQ ID NOS: 49 to 62;
f) an amino acid sequence having 3, 2, or 1 amino acid differences with at least one of the amino acid sequences of SEQ ID NOS: 49 to 62;
g) an amino acid sequence of any one of SEQ ID NOS: 77 to 91;
h) an amino acid sequence having at least 80% identity to at least one of the amino acid sequences of SEQ ID NOS: 77 to 91;
i) an amino acid sequence having 3, 2, or 1 amino acid differences with at least one of the amino acid sequences of SEQ ID NOS: 77 to 91;
   or any suitable combination thereof.

When a single-domain antibody comprises one or more amino acid sequences according to b) and/or c):
i) any amino acid substitution in such an amino acid sequence according to b) and/or c) is preferably a conservative amino acid substitution compared to the corresponding amino acid sequence according to a); and/or
ii) the amino acid sequence according to b) and/or c) preferably only contains amino acid substitutions, and no amino acid deletions or insertions, compared to the corresponding amino acid sequence according to a); and/or
iii) the amino acid sequence according to b) and/or c) may be an amino acid sequence that is derived from an amino acid sequence according to a) by means of affinity maturation using one or more known techniques of affinity maturation.

Similarly, when a single-domain antibody comprises one or more amino acid sequences according to e) and/or f):
i) any amino acid substitution in such an amino acid sequence according to e) and/or f) is preferably a conservative amino acid substitution compared to the corresponding amino acid sequence according to d); and/or
ii) the amino acid sequence according to e) and/or f) preferably only contains amino acid substitutions, and no amino acid deletions or insertions, compared to the corresponding amino acid sequence according to d); and/or
iii) the amino acid sequence according to e) and/or f) may be an amino acid sequence that is derived from an amino acid sequence according to d) by means of affinity maturation using one or more known techniques of affinity maturation.

Also, similarly, when a single-domain antibody comprises one or more amino acid sequences according to h) and/or i):
i) any amino acid substitution in such an amino acid sequence according to h) and/or i) is preferably a conservative amino acid substitution compared to the corresponding amino acid sequence according to g); and/or
ii) the amino acid sequence according to h) and/or i) preferably only contains amino acid substitutions, and no amino acid deletions or insertions, compared to the corresponding amino acid sequence according to g); and/or
iii) the amino acid sequence according to h) and/or i) may be an amino acid sequence that is derived from an amino acid sequence according to g) by means of affinity maturation using one or more known techniques of affinity maturation.

It should be understood that the preceding paragraphs also generally apply to any single-domain antibodies comprising one or more amino acid sequences according to b), c), e), f), h) or i), respectively.

In an embodiment, the single-domain antibody described herein preferably comprises one or more amino acid sequences selected from the group consisting of:
i) an amino acid sequence of any one of SEQ ID NOS: 27 to 40;
ii) an amino acid sequence of any one of SEQ ID NOS: 49 to 62; and
iii) an amino acid sequence of any one of SEQ ID NOS: 77 to 91;
   or any suitable combination thereof.

Also, preferably, in such a single-domain antibody, at least one of the amino acid sequences forms part of the antigen binding site for binding to SARS-CoV-2.

In an embodiment, the present disclosure relates to a single-domain antibody comprising two or more amino acid sequences selected from the group consisting of:
a) an amino acid sequence of any one of SEQ ID NOS: 27 to 40;
b) an amino acid sequence having at least 80% identity to at least one of the amino acid sequences of SEQ ID NOS: 27 to 40;
c) an amino acid sequence having 3, 2, or 1 amino acid differences with at least one of the amino acid sequences of SEQ ID NOS: 27 to 40;
d) an amino acid sequence of any one of SEQ ID NOS: 49 to 62;
e) an amino acid sequence having at least 80% identity to at least one of the amino acid sequences of SEQ ID NOS: 49 to 62;
f) an amino acid sequence having 3, 2, or 1 amino acid differences with at least one of the amino acid sequences of SEQ ID NOS: 49 to 62;
g) an amino acid sequence of at least one of SEQ ID NOS: 77 to 91;
h) an amino acid sequence having at least 80% identity to at least one of the amino acid sequences of SEQ ID NOS: 77 to 91;
i) an amino acid sequence having 3, 2, or 1 amino acid differences with at least one of the amino acid sequences of SEQ ID NOS: 77 to 91;
   such that (i) when the first amino acid sequence corresponds to one of the amino acid sequences according to a), b) or c), the second amino acid sequence corresponds to one of the amino acid sequences according to d), e), f), g), h) or i); (ii) when the first amino acid sequence corresponds to one of the amino acid sequences according to d), e) or f), the second amino acid sequence corresponds to one of the amino acid sequences according to a), b), c), g), h) or i); or (iii) when the first amino acid sequence corresponds to one of the amino acid sequences according to g), h) or i), the second amino acid sequence corresponds to one of the amino acid sequences according to a), b), c), d), e) or f).

In an embodiment, the single-domain antibody described herein preferably comprises two or more amino acid sequences selected from the group consisting of:
i) an amino acid sequence of any one of SEQ ID NOS: 27 to 40;
ii) an amino acid sequence of any one of SEQ ID NOS: 49 to 62; and
iii) an amino acid sequence of any one of SEQ ID NOS: 77 to 91;
   such that, (i) when the first amino acid sequence corresponds to one of the amino acid sequences of SEQ ID NOS: 27 to 40, the second amino acid sequence corresponds to one of the amino acid sequences of SEQ ID NOS: 49 to 62 or of SEQ ID NOS: 77 to 91; (ii) when the first amino acid sequence corresponds to one of the amino acid sequences of SEQ ID NOS: 49 to 62, the second amino acid sequence corresponds to one of the amino acid sequences of SEQ ID NOS: 27 to 40 or of SEQ ID NOS: 77 to 91; or (iii) when the first amino acid sequence corresponds to one of the amino acid sequences of SEQ ID NOS: 77 to 91, the second amino acid sequence corresponds to one of the amino acid sequences of SEQ ID NOS: 27 to 40 or of SEQ ID NOS: 49 to 62.

Also, in such a single-domain antibody, the at least two amino acid sequences preferably form part of the antigen binding site for binding to SARS-CoV-2.

In an embodiment, the present disclosure relates to an a single-domain antibody comprising three or more amino acid sequences, in which the first amino acid sequence is selected from the group consisting of:
a) an amino acid sequence of any one of SEQ ID NOS: 27 to 40;
b) an amino acid sequence having at least 80% identity to at least one of the amino acid sequences of SEQ ID NOS: 27 to 40;
c) an amino acid sequence having 3, 2, or 1 amino acid differences with at least one of the amino acid sequences of SEQ ID NOS: 27 to 40;
   the second amino acid sequence is selected from the group consisting of:
d) an amino acid sequence of any one of SEQ ID NOS: 49 to 62;
e) an amino acid sequence having at least 80% identity to at least one of the amino acid sequences of SEQ ID NOS: 49 to 62;
f) an amino acid sequence having 3, 2, or 1 amino acid differences with at least one of the amino acid sequences of SEQ ID NOS: 49 to 62;
   and the third amino acid sequence is selected from the group consisting of:
g) an amino acid sequence of any one of SEQ ID NOS: 77 to 91;
h) an amino acid sequence having at least 80% identity to at least one of the amino acid sequences of SEQ ID NOS: 77 to 91;
i) an amino acid sequence having 3, 2, or 1 amino acid differences with at least one of the amino acid sequences of SEQ ID NOS: 77 to 91.

Preferably, the first amino acid sequence is selected from the group consisting of an amino acid sequence of any one of SEQ ID NOS: 27 to 40; the second amino acid sequence is selected from the group consisting of an amino acid sequence of any one of SEQ ID NOS: 49 to 62; and the third amino acid sequence is selected from the group consisting of an amino acid sequence of SEQ ID NOS: 77 to 91.

Again, preferably, in such a single-domain antibody, the at least three amino acid sequences form part of the antigen binding site for binding to SARS-CoV-2.

Preferred combinations of such amino acid sequences are described herein.

In an embodiment, in such amino acid sequences, the CDR sequences have at least 70% identity, at least 80% identity, at least 90% identity, at least 91% identity, at least 92% identity, at least 93% identity, at least 94% identity, at least 95% identity, at least 96% identity, at least 97% identity, at least 98% identity, at least 99% identity, or about 100% identity with the CDR sequences of at least one of the amino acid sequences of SEQ ID NOS: 1 to 15. This degree of amino acid identity can, for example, be determined by determining the degree of amino acid identity (in a manner described herein) between said amino acid sequence and one or more of the sequences of SEQ ID NOS: 1 to 15, in which the amino acid residues that form the framework regions are disregarded.

Also, such amino acid sequences are preferably such that they immunospecifically bind to SARS-CoV-2; and more in particular bind to the RBD of SARS-CoV-2.

When the single-domain antibody of the disclosure essentially consists of four framework regions (FR1 to FR4, respectively) and three complementarity determining regions (CDR1 to CDR3, respectively):
CDR1 is preferably selected from the group consisting of:
   a) an amino acid sequence of any one of SEQ ID NOS: 27 to 40;
   b) an amino acid sequence having at least 80% identity with at least one of the amino acid sequences of SEQ ID NOS: 27 to 40;
   c) an amino acid sequence having 3, 2, or 1 amino acid differences with at least one of the amino acid sequences of SEQ ID NOS: 27 to 40;
and/or
CDR2 is preferably selected from the group consisting of:
   d) an amino acid sequence of any one of SEQ ID NOS: 49 to 62;
   e) an amino acid sequence having at least 80% identity with at least one of the amino acid sequences of SEQ ID NOS: 49 to 62;
   f) an amino acid sequence having 3, 2, or 1 amino acid differences with at least one of the amino acid sequences of SEQ ID NOS: 49 to 62;
and/or
CDR3 is preferably selected from the group consisting of:
   g) an amino acid sequence of any one of SEQ ID NOS: 77 to 91;
   h) an amino acid sequence having at least 80% identity with at least one of the amino acid sequences of SEQ ID NOS: 77 to 91;
   i) an amino acid sequence having 3, 2, or 1 amino acid differences with at least one of the amino acid sequences of SEQ ID NOS: 77 to 91.

Preferably, when a single-domain antibody of the disclosure essentially consists of four framework regions (FR1 to FR4, respectively) and three complementarity determining regions (CDR1 to CDR3, respectively), CDR1 is selected from the group consisting of an amino acid sequence of any one of SEQ ID NOS: 27 to 40; and/or CDR2 is selected from the group consisting of an amino acid sequence of any one of SEQ ID NOS: 49 to 62; and/or CDR3 is selected from the group consisting of an amino acid sequence of SEQ ID NOS: 77 to 91.

In particular, when a single-domain antibody essentially consists of four framework regions (FR1 to FR4, respectively) and three complementarity determining regions (CDR1 to CDR3, respectively):
CDR1 is preferably selected from the group consisting of:
   a) an amino acid sequence of any one of SEQ ID NOS: 27 to 40;
   b) an amino acid sequence having at least 80% identity with at least one of the amino acid sequences of SEQ ID NOS: 27 to 40;
   c) an amino acid sequence having 3, 2, or 1 amino acid differences with at least one of the amino acid sequences of SEQ ID NOS: 27 to 40;
and
CDR2 is preferably selected from the group consisting of:
   d) an amino acid sequence of SEQ ID NOS: 49 to 62;
   e) an amino acid sequence having at least 80% identity with at least one of the amino acid sequences of SEQ ID NOS: 49 to 62;
   f) an amino acid sequence having 3, 2, or 1 amino acid differences with at least one of the amino acid sequences of SEQ ID NOS: 49 to 62;
and
CDR3 is preferably selected from the group consisting of:
   g) an amino acid sequence of SEQ ID NOS: 77 to 91;
   h) an amino acid sequence having at least 80% identity with at least one of the amino acid sequences of SEQ ID NOS: 77 to 91;
   i) an amino acid sequence having 3, 2, or 1 amino acid differences with at least one of the amino acid sequences of SEQ ID NOS: 77 to 91.

Preferably, when a single-domain antibody essentially consists of four framework regions (FR1 to FR4, respectively) and three complementarity determining regions (CDR1 to CDR3, respectively), CDR1 is selected from the group consisting of the amino acid sequences of SEQ ID NOS: 27 to 40; and CDR2 is selected from the group consisting of the amino acid sequences of SEQ ID NOS: 49 to 62; and CDR3 is selected from the group consisting of the amino acid sequences of SEQ ID NOS: 77 to 91.

Again, preferred combinations of CDR sequences are described herein.

Also, such single-domain antibodies preferably immunospecifically bind to SARS-CoV-2; and more preferably bind to the RBD of SARS-CoV-2.

In one embodiment, single-domain antibodies described herein (including molecules comprising, or alternatively consisting of, antibody fragments or variants thereof) may immunospecifically bind to the RBD of SARS-CoV-2 (e.g., a polypeptide comprising, or alternatively consisting of, the amino acid sequence of SEQ ID NO: 124 or 127). In an embodiment, single-domain antibodies described herein immunospecifically bind to the RBD of SARS-CoV-2 (e.g., a polypeptide comprising, or alternative consisting of, the amino acid sequence of SEQ ID NO: 124 or 127) and comprise, or alternatively consist of, a VHH domain, CDR1, CDR2, and/or CDR3 corresponding to, or contained within, one or more of SEQ ID NOS: 1 to 15.

In one embodiment, single-domain antibodies (including molecules comprising, or alternatively consisting of, antibody fragments or variants thereof (including derivatives)) may comprise, or alternatively consist of, VHH domains and/or CDRs described herein, which single-domain antibodies immunospecifically bind to SARS-CoV-2 (e.g., the RBD of SARS-CoV-2) and can be assayed for immunospecific binding to SARS-CoV-2 using methods known in the art, for example, the immunoassays disclosed herein.

In an embodiment, a single-domain antibody essentially consists of four framework regions (FR1 to FR4, respectively) and three complementarity determining regions (CDR1 to CDR3, respectively), in which the CDR sequences of said amino acid sequence have at least 70% identity, at least 80% identity, at least 90% identity, at least 91% identity, at least 92% identity, at least 93% identity, at least 94% identity, at least 95% identity, at least 96% identity, at least 97% identity, at least 98% identity, at least 99% identity, or about 100% identity with the CDR sequences of at least one of the amino acid sequences of SEQ ID NOS: 1 to 15. This degree of amino acid identity can, for example, be determined by determining the degree of amino acid identity (in a manner described herein) between said amino acid sequence and one or more of the sequences of SEQ ID NOS: 1 to 15, in which the amino acid residues that form the framework regions are disregarded. Such single-domain antibodies can be as further described herein.

In an embodiment, the framework sequences of the single-domain antibodies described herein may be any suitable framework sequences. Examples of suitable framework sequences will be clear to the skilled person, for example, on the basis the standard handbooks and the further disclosure herein.

The framework sequences are preferably immunoglobulin framework sequences or framework sequences that have been derived from immunoglobulin framework sequences (for example, by humanization or camelization). For example, the framework sequences may be framework sequences derived from a light chain variable domain (e.g., a VL domain sequence) and/or from a heavy chain variable domain (e.g., a VH domain sequence). In some aspects, the framework sequences are either framework sequences that have been derived from a VH domain sequence (in which said framework sequences may optionally have been partially or fully humanized) or are conventional VH domain sequences that have been camelized.

The framework sequences are preferably such that the single-domain antibody of the disclosure comprises, or alternatively consists of, a domain antibody (or an amino acid sequence that is suitable for use as a domain antibody); a single domain antibody (or an amino acid sequence that is suitable for use as a single domain antibody); a “dAb” (or an amino acid sequence that is suitable for use as a dAb); or a VHH sequence.

Preferred, but non-limiting examples of (suitable combinations of) such framework sequences are disclosed herein.

Again, as generally described herein for the single-domain antibodies described herein, it is also possible to use suitable fragments (or combinations of fragments) of any of the foregoing, such as fragments that comprise one or more CDR sequences, flanked by and/or linked via one or more framework sequences (for example, in the same order as these CDRs and framework sequences may occur in the full-sized immunoglobulin sequence from which the fragment has been derived). Such fragments may comprise or can form an immunoglobulin fold, or alternatively be such that they do not comprise or cannot form an immunoglobulin fold.

In one aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also comprise a disulfide bridge, and in particular a disulfide bridge that links the two framework regions that precede and follow the CDR sequence, respectively (for the purpose of forming such a disulfide bridge, cysteine residues that naturally occur in said framework regions may be used, or alternatively cysteine residues may be synthetically added to or introduced into said framework regions). For a further description of these “Expedite fragments”, reference is again made to WO 2003/050531 or WO 2009/127691.

A compound or construct, and in particular a protein or polypeptide (also referred to herein as a “compound described herein” or “polypeptide described herein”, respectively) may comprise or consist essentially of one or more single-domain antibodies described herein (or suitable fragments thereof), and optionally further comprises one or more other groups, residues, moieties or binding units. Further groups, residues, moieties, binding units or amino acid sequences may or may not provide further functionality to the antibodies described herein (and/or to the compound or construct in which it is present) and may or may not modify the properties of the single-domain antibodies described herein.

For example, such further groups, residues, moieties or binding units may be one or more additional amino acid sequences, such that the compound or construct is a fusion protein or fusion polypeptide. In a preferred, but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, dAbs, amino acid sequences that are suitable for use as a dAb, or VHH sequences.

The groups, residues, moieties or binding units may, for example, be chemical groups, residues, moieties, which may or may not by themselves be biologically and/or pharmacologically active. For example, and without limitation, such groups may be linked to the one or more single-domain antibodies described herein so as to provide a “derivative” of an amino acid sequence or polypeptide described herein, as further described herein.

Also provided are compounds or constructs, that comprise or consist essentially of one or more derivatives as described herein, and optionally further comprise one or more other groups, residues, moieties or binding units, optionally linked via one or more linkers. Preferably, said one or more other groups, residues, moieties or binding units are amino acid sequences.

In the compounds or constructs described above, the one or more single-domain antibodies described herein and the one or more groups, residues, moieties or binding units may be linked directly to each other and/or via one or more suitable linkers or spacers. For example, when the one or more groups, residues, moieties or binding units are amino acid sequences, the linkers may also be amino acid sequences, so that the resulting compound or construct is a fusion protein or fusion polypeptide.

The single-domain antibodies described herein can be used as “building blocks” to form polypeptides described herein, e.g., by combining them with other groups, residues, moieties or binding units, in order to form compounds or constructs as described herein (e.g., biparatopic, bi/multivalent and/or bi/multispecific polypeptides described herein) which combine within one molecule one or more desired properties or biological functions.

The compounds or polypeptides described herein can generally be prepared by a method which comprises at least one step of linking the one or more single-domain antibodies described herein to the one or more further groups, residues, moieties or binding units, optionally via the one or more suitable linkers, so as to provide the compound or polypeptide described herein.

Polypeptides described herein can also be prepared by a method which generally comprises at least the steps of providing a nucleic acid that encodes a polypeptide described herein, expressing said nucleic acid in a suitable manner, and recovering the expressed polypeptide described herein. Such methods can be performed by any suitable manner, which will be clear to the skilled person, for example on the basis of the methods and techniques further described herein.

In an embodiment, a compound or a polypeptide described herein may have an increased half-life, compared to the corresponding single-domain antibody described herein. Some preferred, but non-limiting, examples of such compounds and polypeptides will become clear to the skilled person based on the further disclosure herein, and, for example, comprise amino acid sequences or polypeptides described herein that have been chemically modified to increase the half-life thereof (for example, by means of pegylation); single-domain antibodies described herein that comprise at least one additional binding site for binding to a serum protein (e.g., serum albumin); or polypeptides comprising at least one single-domain antibody described herein that is linked to at least one moiety (and in particular at least one amino acid sequence) that increases the half-life of the single-domain antibody described herein. Examples of polypeptides described herein that comprise such half-life extending moieties or amino acid sequences will become clear to the skilled person based on the further disclosure herein; and for example include, without limitation, polypeptides in which the one or more single-domain antibodies of the disclosure are linked to one or more serum proteins or fragments thereof (e.g., (human) serum albumin or suitable fragments thereof) or to one or more binding units that can bind to serum proteins (e.g., for example, domain antibodies, amino acid sequences that are suitable for use as a domain antibody, single domain antibodies, amino acid sequences that are suitable for use as a single domain antibody, dAbs, amino acid sequences that are suitable for use as a dAb, or VHH sequences that can bind to serum proteins such as serum albumin (e.g., human serum albumin), serum immunoglobulins such as IgG, or transferrin; polypeptides in which a single-domain antibodies described herein is linked to an Fc portion (e.g., a human Fc) or a suitable part or fragment thereof; or polypeptides in which the one or more single-domain antibodies described herein are suitable linked to one or more small proteins or peptides that can bind to serum proteins (e.g., the proteins and peptides described in for example, WO 91/01743, WO 01/45746, and WO 02/076489).

Generally, the compounds or polypeptides described herein with increased half-life preferably have a half-life that is at least 1.5 times, preferably at least 2 times, such as at least 5 times, for example at least 10 times or more than 20 times, greater than the half-life of the corresponding single-domain antibody described herein. For example, the compounds or polypeptides described herein with increased half-life may have a half-life that is increased with more than 1 hours, preferably more than 2 hours, more preferably more than 6 hours, such as more than 12 hours, or even more than 24, 48 or 72 hours, compared to the corresponding single-domain antibody described herein.

The compounds or polypeptides described herein may have a serum half-life that is increased with more than 1 hours, preferably more than 2 hours, more preferably more than 6 hours, such as more than 12 hours, or even more than 24, 48 or 72 hours, compared to the corresponding single-domain antibody described herein.

The compounds or polypeptides described herein may exhibit a serum half-life in human of at least about 12 hours, preferably at least 24 hours, more preferably at least 48 hours, even more preferably at least 72 hours or more. For example, compounds or polypeptides described herein may have a half-life of at least 5 days (e.g., about 5 to 10 days), preferably at least 9 days (e.g., about 9 to 14 days), more preferably at least about 10 days (e.g., about 10 to 15 days), or at least about 11 days (e.g., about 11 to 16 days), more preferably at least about 12 days (e.g., about 12 to 18 days or more), or more than 14 days (e.g., about 14 to 19 days).

Single-domain antibodies and antibody fragments or variants (including derivatives) described herein may include, for example, one or more amino acid sequence alterations (addition, deletion, substitution and/or insertion of an amino acid residue). These alterations may be made in one or more framework regions and/or one or more CDRs. The single-domain antibodies described herein (including antibody fragments, and variants and derivative thereof) can be routinely made by methods known in the art. Molecules comprising, or alternatively consisting of, fragments or variants of any of the VHH domains and CDRs whose sequences are specifically disclosed herein may be employed in accordance with the present disclosure. Nucleic acid molecules encoding these single-domain antibodies and molecules (including fragments, variants, and derivatives) are also provided by the present disclosure.

In one embodiment, panels of single-domain antibodies (including molecules comprising, or alternatively consisting of, antibody fragments or variants) are described, wherein the panel members correspond to one, two, three, four, five, ten, fifteen, twenty, or more different antibodies described herein (e.g., whole antibodies, Fabs, F(ab′)2 fragments, Fd fragments, disulfide-linked Fvs (sdFvs), antiidiotypic (anti-Id) antibodies, and scFvs). The present disclosure further provides mixtures of antibodies, wherein the mixture corresponds to one, two, three, four, five, ten, fifteen, twenty, or more different antibodies of the disclosure (e.g., whole antibodies, Fabs, F(ab′)2 fragments, Fd fragments, disulfide-linked Fvs (sdFvs), antiidiotypic (anti-Id) antibodies, and scFvs).

In one embodiment, compositions may comprise or consist of one, two, three, four, five, ten, fifteen, twenty, or more single-domain antibodies described herein (including molecules comprising, or alternatively consisting of, antibody fragments or variants thereof). A composition described herein may comprise, or alternatively consist of, one, two, three, four, five, ten, fifteen, twenty, or more amino acid sequences of one or more single-domain antibodies or fragments or variants thereof. Alternatively, a composition described herein may comprise, or alternatively consist of, nucleic acid molecules encoding one or more single-domain antibodies of the disclosure.

In one embodiment, fusion proteins may comprise a single-domain antibody (including molecules comprising, or alternatively consisting of, antibody fragments or variants thereof) described herein, and a heterologous polypeptide (e.g., a polypeptide unrelated to an antibody or antibody domain). Nucleic acid molecules encoding these fusion proteins are also provided by the present disclosure. A composition described herein may comprise, or alternatively consist of, one, two, three, four, five, ten, fifteen, twenty or more fusion proteins of the disclosure. Alternatively, a composition described herein may comprise, or alternatively consist of, nucleic acid molecules encoding one, two, three, four, five, ten, fifteen, twenty or more fusion proteins of the disclosure.

In one embodiment, a recombinant nucleic acid molecule, generally isolated, encoding a single-domain antibody (including molecules which may comprise or consist of an antibody fragment or variant thereof) described herein is provided. The present disclosure also provides a host or host cell transformed with a nucleic acid molecule described herein and progeny thereof. The present disclosure also provides a method for the production of a single-domain antibody (including a molecule comprising, or alternatively consisting of, an antibody fragment or variant thereof) described herein. The present disclosure further provides a method of expressing a single-domain antibody (including a molecule comprising, or alternatively consisting of, an antibody fragment or variant thereof) described herein from a recombinant nucleic acid molecule. These and other aspects of the present disclosure are described in further detail below.

In one embodiment, methods and compositions for detecting, diagnosing and/or prognosing coronavirus infections, preferably SARS-CoV-2 infections (COVID-19), in an animal, preferably a mammal, and most preferably a human, may comprise using single-domain antibodies (including molecules which comprise, or alternatively consist of, antibody fragments or variants thereof) that immunospecifically bind to SARS-CoV-2 (e.g., the RBD of SARS-CoV-2). Diseases and disorders which can be detected, diagnosed or prognosed with the single-domain antibodies described herein include, but are not limited to, COVID-19, acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

In one embodiment, methods and compositions for preventing, treating or ameliorating coronavirus infections, preferably SARS-CoV-2 infections (COVID-19), in an animal, preferably a mammal, and most preferably a human, may comprise administering to said animal an effective amount of one or more single-domain antibodies (including molecules which comprise, or alternatively consist of, antibody fragments or variants thereof) that immunospecifically bind to SARS-CoV-2 (e.g., the RBD of SARS-CoV-2). Diseases and disorders which can be prevented, treated or inhibited by administering an effective amount of one or more single-domain antibodies or molecules described herein include, but are not limited to, COVID-19, acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

### BRIEF DESCRIPTION OF THE DRAWINGS

For a further understanding of the nature, objects, and advantages described herein, reference should be had to the following detailed description, read in conjunction with the following drawings, wherein like reference numerals denote like elements.

FIGS. 1A-1F show affinity assay results for six exemplary single-domain antibodies described herein. FIG. 1A shows affinity assay results for exemplary single-domain antibody Nb15 (SEQ ID NO: 1). FIG. 1B shows affinity assay results for exemplary single-domain antibody msNb12 (SEQ ID NO: 13). FIG. 1C shows affinity assay results for exemplary single-domain antibody Nb17 (SEQ ID NO: 2). FIG. 1D shows affinity assay results for exemplary single-domain antibody Nb19 (SEQ ID NO: 3). FIG. 1E shows affinity assay results for exemplary single-domain antibody Nb56 (SEQ ID NO: 4). FIG. 1F shows affinity assay results for exemplary single-domain antibody mNb30 (SEQ ID NO: 14).

FIGS. 2A-2F show affinity assay results for six exemplary single-domain antibody Fc fusion constructs described herein. FIG. 2A shows affinity assay results for mNb30-Fc monomer (SEQ ID NO: 117). FIG. 2B shows affinity assay results for Nb15-Fc trimer (SEQ ID NO: 118). FIG. 2C shows affinity assay results for Nb17-Fc trimer (SEQ ID NO: 119). FIG. 2D shows affinity assay results for Nb19-Fc trimer (SEQ ID NO: 120). FIG. 2E shows affinity assay results for Nb56-Fc trimer (SEQ ID NO: 121). FIG. 2F shows affinity assay results for msNb12-Fc trimer (SEQ ID NO: 122).

FIGS. 3A & 3B show antibody titers from a llama immunized with both SARS-CoV-2 RBD and spike polypeptides (SEQ ID NO: 129 and SEQ ID NO: 130, respectively). Good immune responses against SARS-CoV-2 RBD (FIG. 3A) and SARS-CoV-2 Spike (FIG. 3B) were obtained within 24-38 days post-immunization.

FIGS. 4A & 4B show antibody titers from transgenic mice expressing camelid antibody genes immunized with both SARS-CoV-2 RBD and spike polypeptides (SEQ ID NO: 129 and SEQ ID NO: 130, respectively) (FIG. 4A) or with SARS-CoV-2 spike polypeptide alone (SEQ ID NO: 130) (FIG. 4B).

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

### Definitions

As used herein, the term “SARS-CoV-2” refers broadly to any SARS-CoV-2 sequence unless indicated otherwise, including wild-type SARS-CoV-2 and any variant or mutant SARS-CoV-2 sequence. Examples of certain known SARS-CoV-2 variants are provided herein but the disclosure is not limited to these SARS-CoV-2 variants except where indicated. Likewise, the terms “SARS-CoV-1”, “MERS-CoV”, “HCoV-OC43”, “HCoV-HKU1”, “HCoV-NL63”, and “HCoV-229E” refers broadly to wild-type and any variants or mutants thereof unless indicated otherwise.

As used herein, the terms “antibody that immunospecifically binds SARS-CoV-2” “antibody that specifically binds SARS-CoV-2” and “anti-SARS-CoV-2 antibody” refers broadly to an antibody that is capable of binding to SARS-CoV-2, preferably to the receptor binding domain (“RBD”) of SARS-CoV-2.

An amino acid sequence (e.g., a single-domain antibody, a polypeptide described herein, or generally an antigen binding protein or polypeptide or a fragment thereof) that can specifically (e.g., immunospecifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.

As used herein, the term “specificity” refers broadly to the number of different types of antigens or antigenic determinants to which a particular antigen-binding molecule or antigen-binding protein (e.g., a single-domain antibody or a polypeptide described herein) molecule can bind. The specificity of an antigen-binding protein can be determined based on affinity and/or avidity which can be measured, for example, using known techniques for measuring binding between an antigen-binding molecule (e.g., a single-domain antibody or polypeptide described herein) and the pertinent antigen. Typically, antigen-binding proteins (e.g., the single-domain antibodies and/or polypeptides described herein) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter. Any K_{D} value greater than 10⁻⁴ moles/liter is generally considered to indicate non-specific binding. Preferably, antigen-binding proteins (e.g., the single-domain antibodies and/or polypeptides described herein) will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (RIA), enzyme immunoassays (EIA) and sandwich competition assays, and the different variants thereof known in the art; as well as the other techniques mentioned herein. The dissociation constant may be the actual or apparent dissociation constant. Methods for determining the dissociation constant will be clear to the skilled person and are known in the art.

As used herein, an antibody that immunospecifically binds to the RBD of SARS-CoV-2 has the property of binding to the RBD of SARS-CoV-2 such that a coronavirus, preferably SARS-CoV-2 virus, is inactivated (“neutralized”). An anti-SARS-CoV-2 antibody candidate can be tested for such activity, for example, by adsorbing anti-SARS-CoV-2 RBD antibody to immobilized RBD of SARS-CoV-2 followed by subjecting the adsorbed antibody to elution with an excess of isolated ACE2 (angiotensin converting enzyme 2) polypeptides. If an eluent comprising an excess of the ACE2 polypeptides produces an eluate comprising a greater concentration of the candidate antibody than the concentration of candidate antibody present in an eluate produced by a “blank” eluent (the same eluent comprising no ACE2) in a control elution, as determined by, e.g., radioimmunoassays performed on the respective eluates with radiolabelled, soluble SARS-CoV-2 RBD, then the candidate antibody competes with the ACE2 polypeptides for binding to SARS-CoV-2 RBD and accordingly, impairs or eliminates the binding of SARS-CoV-2 RBD to ACE2.

As used herein, an anti-SARS-CoV-2 RBD antibody with the property or capability of “neutralizing SARS-CoV-2,” refers broadly to as an anti-SARS-CoV-2 RBD antibody capable of reducing or inhibiting the activity of coronaviruses, preferably SARS-CoV-2. An anti-SARS-CoV-2 RBD antibody candidate can be tested for such activity, for example, by measuring prevention of SARS-CoV-2 infection and/or activity in one or more biological assays. The anti-SARS-CoV-2 RBD antibodies may bind an epitope contained with the amino acid sequences of SEQ ID NOS: 124, 125, 126, 127, 128, or a combination thereof.

As used herein, “coronavirus,” refers broadly to viruses that are members of the coronavirus group. Coronaviruses are named for the crown-like spikes on their surface. There are four main sub-groupings of coronaviruses, known as alpha, beta, gamma, and delta. Preferred coronavirus include but are not limited to SARS-CoV-1, MERS-CoV, SARS-CoV-2, HCoV-OC43, HCoV-HKU1, HCoV-NL63, HCoV-229E or a combination thereof.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity.

“Native antibodies and immunoglobulins” or “conventional antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers broadly to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The term “single variable domain” or “immunoglobulin single variable domain”, defines molecules wherein the antigen binding site is present on, and formed by, a single immunoglobulin domain. This sets single variable domains apart from “conventional” immunoglobulins or their fragments, wherein two immunoglobulin domains, in particular two “variable domains” interact to form an antigen binding site. As described above, in conventional immunoglobulins, typically, a heavy chain variable domain (VH) and a light chain variable domain (VL) interact to form an antigen binding site. In this case, the complementarity determining regions (CDRs) of both VH and VL will contribute to the antigen binding site, e.g., a total of 6 CDRs will be involved in antigen binding site formation.

In contrast, the binding site of an immunoglobulin single variable domain is formed by a single VH or VL domain. Hence, the antigen binding site of an immunoglobulin single variable domain is formed by no more than three CDRs. The term “immunoglobulin single variable domain” does comprise fragments of conventional immunoglobulins wherein the antigen binding site is formed by a single variable domain.

Generally, immunoglobulin single variable domains will be amino acid sequences that consist essentially of four framework regions (FR1, FR2, FR3, FR4) and three complementarity determining regions (CDR1, CDR2, CDR3); or any suitable fragment of such an amino acid sequence (which will then usually comprise at least some of the amino acid residues that form at least one of the CDRs). Such immunoglobulin single variable domains and fragments are most preferably such that they comprise an immunoglobulin fold or are capable of forming, under suitable conditions, an immunoglobulin fold. As such, the immunoglobulin single variable domain may, for example, comprise a light chain variable domain sequence (e.g., a VL domain sequence) or a suitable fragment thereof; or a heavy chain variable domain sequence (e.g., a VH domain sequence or VHH domain sequence) or a suitable fragment thereof; as long as it is capable of forming a single antigen binding unit (e.g., a functional antigen binding unit that essentially consists of the immunoglobulin single variable domain, such that the single antigen binding domain does not need to interact with another variable domain to form a functional antigen binding unit, as is the case for the variable domains that are present in, for example, conventional antibodies and scFv fragments that need to interact with another variable domain (e.g., through a VH/VL interaction) to form a functional antigen binding domain).

In an embodiment, the immunoglobulin single variable domains are light chain variable domain sequences (e.g., a VL domain sequence) or heavy chain variable domain sequences (e.g., a VH domain sequence). More specifically, the single variable domains can be heavy chain variable domain sequences that are derived from a conventional four-chain antibody or heavy chain variable domain sequences that are derived from a heavy chain antibody.

The immunoglobulin single variable domain may be a domain antibody (or an amino acid sequence that is suitable for use as a domain antibody), a single-domain antibody (or an amino acid sequence that is suitable for use as a single-domain antibody), a “dAb” (or an amino acid sequence that is suitable for use as a dAb), a VHH domain sequence, other immunoglobulin single variable domains, or any suitable fragment of any one thereof. For a general description of (single) domain antibodies, reference is made to the art cited herein, as well as to EP 0 368 684. For the term “dAb's”, reference is made to Ward et al. 1989 (Nature 341: 544-546), Holt et al. 2003 (Trends Biotechnol. 21: 484-490); as well as to, for example, WO 04/068820, WO 06/030220, and WO 06/003388. It should also be noted that, although less preferred in the context described herein because they are not of mammalian origin, immunoglobulin single variable domains can be derived from certain species of shark (for example, “IgNAR domains”, see, for example, WO 05/18629).

Papain digestion of conventional antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

“Fv” is the minimum antibody fragment that contains a complete antigen-recognition and binding site. In a two-chain Fv species, this region consists of a dimer of one heavy and one light chain variable domain in tight, non-covalent association. In a single-chain Fv species, one heavy and one light chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, a single variable domain (or half of an Fv comprising only three CDRs specific for an antigen) also can have the ability to recognize and bind antigen as described herein.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (K) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1, and IgA2. The heavy-chain constant domains that correspond to the different classes of immunoglobulins are called α, δ, ε, γ, and μ, respectively. The subunit structures and three-dimensional configurations of different classes of immunoglobulins are well known. “Therapeutic Antibody Engineering” (1^{st} Ed.) Strohl & Strohl Woodhead Publishing (2012).

“Antibody fragments” comprise a portion of an intact antibody, generally the antigen binding or variable region of the intact antibody. Examples of antibody fragments include Fab, Fab′, F(ab′)2, and Fv fragments; diabodies; single-chain antibody molecules, including single-chain Fv (scFv) molecules; and multispecific antibodies formed from antibody fragments. “Human Monoclonal Antibodies: Methods and Protocols” (2^{nd} Ed.) Steinitz (Ed.) Humana Press (2019).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

“Humanized” forms of non-human (e.g., murine) antibodies are chimeric immunoglobulins, immunoglobulin chains or fragments thereof (e.g., Fv, Fab, Fab′, F(ab′) 2 or other antigen-binding subsequences of antibodies) which comprise minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (e.g., mouse, rat, rabbit, or camelid) or a synthetic sequence (donor antibody), having the desired specificity, affinity, and capacity. In some instances, Fv framework region (FR) residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In an embodiment, all the CDRs are from the donor immunoglobulin in a humanized immunoglobulin. Constant regions need not be present, but if they are, they should be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions that have substantially no effect on antigen binding or other immunoglobulin functions. Humanized immunoglobulins can be constructed by means of genetic engineering. See for example, U.S. Pat. No. 5,585,089.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains, which enables the scFv to form the desired structure for antigen binding. For a review of scFv, see Pluckthun, in The Pharmacology of Monoclonal Antibodies, vol. 113, Rosenburg and Moore eds., Springer-Verlag, New York, pp. 269-315 (1994).

The term “diabodies” refers broadly to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “variant” as used herein refers broadly to a polypeptide that possesses a similar or identical function as a SARS-CoV-2 polypeptide (e.g., a SARS-CoV-2 RBD polypeptide), an anti-SARS-CoV-2 antibody, or antibody fragment thereof, but does not necessarily comprise a similar or identical amino acid sequence of a SARS-CoV-2 polypeptide, anti-SARS-CoV-2 antibody, or antibody fragment thereof, or possess a similar or identical structure of a SARS-CoV-2 polypeptide, an anti-SARS-CoV-2 antibody, or antibody fragment thereof. A variant having a similar amino acid identity refers broadly to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a SARS-CoV-2 polypeptide, an anti-SARS-CoV-2 antibody or antibody fragment thereof (including a VHH domain or CDR having an amino acid sequence of any one of those described herein); (b) a polypeptide encoded by a nucleotide sequence, the complementary sequence of which hybridizes under stringent conditions to a nucleotide sequence encoding a SARS-CoV-2 polypeptide or fragment thereof, an anti-SARS-CoV-2 antibody or antibody fragment thereof (including a VHH domain or CDR having an amino acid sequence of any one of those described herein), of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or about 100% identical to the nucleotide sequence encoding a SARS-CoV-2 polypeptide or fragment thereof, an anti-SARS-CoV-2 antibody or antibody fragment thereof (including a VHH domain or CDR having an amino acid sequence described herein). A polypeptide with similar structure to a SARS-CoV-2 polypeptide or fragment thereof, an anti-SARS-CoV-2 antibody or antibody fragment thereof, described herein refers broadly to a polypeptide that has a similar secondary, tertiary or quaternary structure of a SARS-CoV-2 polypeptide or fragment thereof, an anti-SARS-CoV-2 antibody, or antibody fragment thereof, described herein. The structure of a polypeptide can determined by methods known to those skilled in the art, including but not limited to, X-ray crystallography, nuclear magnetic resonance, and crystallographic electron microscopy. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (e.g., % identity=number of identical overlapping positions/total number of positions ×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268 (1990), modified as in Karlin and Altschul Proc. Natl. Acad. Sci. USA 90:5873-5877 (1993). The BLASTn and BLASTx programs of Altschul, et al. J. Mal. Biol. 215:403-410(1990) have incorporated such an algorithm. BLAST nucleotide searches can be performed with the BLASTn program, score=100, wordlength=12 to obtain nucleotide sequences homologous to a nucleic acid molecule described herein. BLAST protein searches can be performed with the BLASTx program, score=50, wordlength=3 to obtain amino acid sequences homologous to a protein molecule described herein. To obtain gapped alignments for comparison purposes, Gapped BLAST can be utilized as described in Altschul et al. Nucleic Acids Res. 25:3389-3402(1997). Alternatively, PSI-BLAST can be used to perform an iterated search, which detects distant relationships between molecules (Id.). When utilizing BLAST, Gapped BLAST, and PSI-BLAST programs, the default parameters of the respective programs (e.g., BLASTx and BLASTn) can be used. (See ncbi.nlm.nih.gov).

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. App. Biosci., 10.3-5(1994); and FASTA described in Pearson and Lipman Proc. Natl. Acad. Sci. 85:2444-8(1988). Within FASTA, ktup is a control option that sets the sensitivity and speed of the search.

“Conservative” amino acid substitutions are those substitutions that do not substantially affect or decrease the affinity of a protein, such as an antibody to SARS-CoV-2. For example, a single-domain antibody that immunospecifically binds SARS-CoV-2 can include at most about 1, at most about 2, at most about 5, at most about 10, at most about 15, at most about 20, or at most about 25 conservative substitutions and immunospecifically bind a SARS-CoV-2 polypeptide. The term “conservative variant” also includes the use of a substituted amino acid in place of an unsubstituted parent amino acid, provided that antibody immunospecifically binds SARS-CoV-2. Non-conservative substitutions are those that reduce an activity or binding to SARS-CoV-2.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “derivative” as used herein, refers broadly to a variant polypeptide described herein that comprises, or alternatively consists of, an amino acid sequence of a SARS-CoV-2 polypeptide or fragment thereof, or an antibody described herein that immunospecifically binds to SARS-CoV-2, which has been altered by the introduction of amino acid residue substitutions, deletions or additions. The term “derivative” as used herein also refers broadly to a SARS-CoV-2 polypeptide or fragment thereof, or an antibody that immunospecifically binds to SARS-CoV-2 which has been modified, e.g., by the covalent attachment of any type of molecule to the polypeptide. For example, but not by way of limitation, a SARS-CoV-2 polypeptide or fragment thereof, or an anti-SARS-CoV-2 antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a SARS-CoV-2 polypeptide or fragment thereof, or an anti-SARS-CoV-2 antibody or fragment thereof, may be modified by chemical modifications using techniques known to those of skill in the art, including, but not limited to, specific chemical cleavage, acetylation, formylation, metabolic synthesis of tunicamycin, etc. Further, a derivative of a SARS-CoV-2 polypeptide or fragment thereof, or an anti-SARS-CoV-2 antibody or fragment thereof, may comprise one or more non-classical amino acids. A polypeptide derivative possesses a similar or identical function as a SARS-CoV-2 polypeptide or fragment thereof, or an anti-SARS-CoV-2 antibody or fragment thereof, described herein.

The term “epitopes” as used herein refers broadly to portions of SARS-CoV-2 having antigenic or immunogenic activity in an animal, preferably a mammal. An epitope having immunogenic activity is a portion of SARS-CoV-2 that elicits an antibody response in an animal. An epitope having antigenic activity is a portion of SARS-CoV-2 to which an antibody immunospecifically binds as determined by any method known in the art, for example, by the immunoassays described herein. Antigenic epitopes need not necessarily be immunogenic.

The term “fragment” as used herein refers broadly to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of SARS-CoV-2, or an antibody that immunospecifically binds to SARS-CoV-2.

The terms “fusion protein” or “fusion polypeptide” as used herein refers broadly to a polypeptide that comprises, or alternatively consists of, an amino acid sequence of an anti-SARS-CoV-2 antibody described herein and an amino acid sequence of a heterologous polypeptide (e.g., a polypeptide unrelated to an antibody or antibody domain).

The term “host cell” as used herein refers broadly to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

“Treatment” refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this disclosure.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

### Anti-SARS-CoV-2 Antibodies

In addition to conventional monoclonal antibodies (mAbs), heavy-chain-only antibodies (HCAbs) isolated from camelids provide an alternative for development of therapeutic antibodies. HCAbs consist of only two heavy chains without light chains, thereby only comprising a single variable domain (VHH) referred to as a single-domain antibody. In addition to having affinities and specificities for antigens similar to those of traditional antibodies, single-domain antibodies are smaller in size and show higher stability than most antibodies. Due to their structure, single-domain antibodies can be easily constructed into multivalent or multispecific formats and produced with convenient steps of purification at low manufacturing cost. Furthermore, single-domain antibodies can be easily nebulized and delivered directly to lungs via an inhaler, which make them particularly promising for development of neutralizing antibodies targeting respiratory pathogens such as SARS-CoV-2. Inhaled formulations allow for easier administration outside the hospital, at earlier stages of disease, which is very important to combat the COVID-19 pandemic.

In accordance with the terminology used in the art described above, the variable domains present in naturally occurring heavy chain antibodies are referred to herein as “VHH domains” in order to distinguish them from the heavy chain variable domains that are present in conventional four-chain antibodies (which are referred to herein as “VH domains”) and from the light chain variable domains that are present in conventional four-chain antibodies (which are referred to herein as “VL domains”).

As described herein, VHH domains have a number of distinct structural characteristics and functional properties which make isolated VHH domains and proteins comprising the same highly advantageous for use as functional antigen-binding domains or proteins. In particular, and without being limited thereto, VHH domains (which have been “designed” by nature to functionally bind to an antigen without the presence of, and without any interaction with, a light chain variable domain) can function as a single, relatively small, functional antigen-binding structural unit, domain or protein. This distinguishes VHH domains from VH and VL domains of conventional four-chain antibodies, which by themselves are generally not suited for practical application as single antigen-binding proteins or domains, but need to be combined in some form or another to provide a functional antigen-binding unit (for example, conventional antibody fragments such as Fab fragments or scFv fragments, which consist of a VH domain covalently linked to a VL domain).

Because of these distinct properties, the use of VHH domains as single antigen-binding proteins or as antigen-binding domains (e.g., as part of a larger protein or polypeptide) offers a number of significant advantages over the use of conventional VH and VL domains, scFvs or conventional antibody fragments (e.g., Fab- or F(ab′)₂-fragments).

In an embodiment, the present disclosure provides single-domain antibodies that immunospecifically bind SARS-CoV-2, and in particular single-domain antibodies that bind the receptor binding domain (RBD) of SARS-CoV-2; as well as proteins and/or polypeptides comprising at least one such single-domain antibody.

In an embodiment, the present disclosure provides single-domain antibodies that immunospecifically bind SARS-CoV-2, and proteins and/or polypeptides comprising the same, that have improved therapeutic and/or pharmacological properties and/or other advantageous properties (for example, improved ease of preparation and/or reduced costs of goods), compared to conventional antibodies against SARS-CoV-2 or fragments thereof, compared to constructs that could be based on such conventional antibodies or antibody fragments (e.g., Fab′ fragments, F(ab′)₂ fragments, scFv constructs, “diabodies” and other multispecific constructs (see, for example, Holliger and Hudson, Nat Biotechnol. 2005 September; 23(9):1126-36)), and also compared to the so-called “dAbs” or similar (single) domain antibodies that may be derived from variable domains of conventional antibodies. These improved and advantageous properties are described herein, and for example include, without limitation, one or more of:
increased affinity and/or avidity for SARS-CoV-2, either in a monovalent format, in a multivalent format (for example in a bivalent format) and/or in a multispecific format (e.g., one of the multispecific formats described herein);
better suitability for formatting in a multivalent format (e.g., in a bivalent format);
better suitability for formatting in a multispecific format (e.g., one of the multispecific formats described herein);
improved suitability or susceptibility for “humanizing” substitutions (as described herein);
less immunogenicity, either in a monovalent format, in a multivalent format (e.g., in a bivalent format) and/or in a multispecific format (e.g., one of the multispecific formats described herein);
increased stability, either in a monovalent format, in a multivalent format (e.g., in a bivalent format) and/or in a multispecific format (e.g., one of the multispecific formats described herein);
increased specificity towards SARS-CoV-2, either in a monovalent format, in a multivalent format (e.g., in a bivalent format) and/or in a multispecific format (e.g., one of the multispecific formats described herein);
decreased or, where desired, increased cross-reactivity with other viruses, such as other coronaviruses;
   and/or
one or more other improved properties desirable for pharmaceutical use (including prophylactic use and/or therapeutic use) and/or for diagnostic use (including, but not limited to, use for diagnostic assays), either in a monovalent format, in a multivalent format (e.g., in a bivalent format) and/or in a multispecific format (e.g., one of the multispecific formats described herein).

As generally described herein for the amino acid sequences described herein, single-domain antibodies described herein are preferably in essentially isolated form (as described herein), or form part of a protein or polypeptide (as described herein), which may comprise or consist essentially of one or more single-domain antibodies described herein and which may optionally further comprise one or more further amino acid sequences (all optionally linked via one or more suitable linkers). For example, and without limitation, the one or more amino acid sequences described herein may be used as a binding unit in such a protein or polypeptide, which may optionally comprise one or more further amino acid sequences that can serve as a binding unit (e.g., against one or more targets other than SARS-CoV-2), so as to provide a monovalent, multivalent or multispecific polypeptide described herein, all as described herein. In particular, such a protein or polypeptide may comprise or consist essentially of one or more single-domain antibodies described herein and optionally one or more other single-domain antibodies (e.g., directed against targets other than SARS-CoV-2), all optionally linked via one or more suitable linkers, so as to provide a monovalent, multivalent or multispecific single-domain antibody construct, as further described herein. Such proteins or polypeptides may also be in essentially isolated form (as described herein).

In a single-domain antibody described herein, the binding site for SARS-CoV-2 is preferably formed by the CDR sequences. Optionally, a single-domain antibody described herein may also, and in addition to the at least one binding site for binding against SARS-CoV-2 comprise one or more further binding sites for binding against other antigens, proteins or targets. For methods and positions for introducing such second binding sites, reference is made to, for example, Keck and Huston, Biophysical Journal, 71, October 1996, 2002-2011; EP 0 640 130; and WO 06/07260.

As generally described herein for the amino acid sequences described herein, the single-domain antibodies described herein may generally be directed against any antigenic determinant, epitope, part, domain, subunit or conformation (where applicable) of SARS-CoV-2.

As already described herein, the amino acid sequence and structure of a single-domain antibody can be considered—without being limited thereto—to be comprised of four framework regions or “FRs” (or sometimes also referred to as “FW's”), which are referred to in the art and herein as “Framework region 1” or “FR1”; as “Framework region 2” or “FR2”; as “Framework region 3” or “FR3”; and as “Framework region 4” or “FR4”; which framework regions are interrupted by three complementary determining regions or “CDRs”, which are referred to in the art as “Complementarity Determining Region 1” or “CDR1”; as “Complementarity Determining Region 2” or “CDR2”; and as “Complementarity Determining Region 3” or “CDR3”. Some preferred framework sequences and CDRs (and combinations thereof) that are present in the single-domain antibodies are as described herein. Other suitable CDR sequences can be obtained by the methods described herein.

According to a non-limiting but preferred aspect, the CDR sequences present in the single-domain antibodies described herein are such that:
the single-domain antibodies can bind to SARS-CoV-2 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter or less;
   and/or such that:
the single-domain antibodies can bind to SARS-CoV-2 with a kₒₙ rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹; and/or such that:
the single-domain antibodies can bind to SARS-CoV-2 with a k_{off} rate between 1 s⁻¹ (t_{1/2}=0.69s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, the CDR sequences present in the single-domain antibodies described herein are such that a monovalent single-domain antibody described herein (or a polypeptide that contains only one single-domain antibody described herein) is preferably such that it will bind to SARS-CoV-2 with an affinity less than 500 nM, preferably less than 200 nM, more preferably less than 10 nM, such as less than 500 pM.

The affinity of the single-domain antibody described herein to SARS-CoV-2 can be determined in any suitable manner, for example, using the general techniques for measuring K_{D}, k_{off} or kₒₙ described herein, as well as some of the specific assays described herein.

Some preferred IC₅₀ values for binding of the single-domain antibodies described herein (and of polypeptides comprising the same) to SARS-CoV-2 are described herein.

In an embodiment, a single-domain antibody (as described herein) that immunospecifically binds to SARS-CoV-2 is provided, consisting of four framework regions (FR1, FR2, FR3, FR4) and three complementarity determining regions (CDR1, CDR2 CDR3), in which:
CDR1 is selected from the group consisting of:
   a) an amino acid sequence of any one of SEQ ID NOS: 27 to 40;
   b) an amino acid sequence having at least 80% identity with at least one of the amino acid sequences of SEQ ID NOS: 27 to 40;
   c) an amino acid sequence having 3, 2, or 1 amino acid differences with at least one of the amino acid sequences of SEQ ID NOS: 27 to 40;
and/or
CDR2 is selected from the group consisting of:
   d) an amino acid sequence of any one of SEQ ID NOS: 49 to 62;
   e) an amino acid sequence having at least 80% identity with at least one of the amino acid sequences of SEQ ID NOS: 49 to 62;
   f) an amino acid sequence having 3, 2, or 1 amino acid differences with at least one of the amino acid sequences of SEQ ID NOS: 49 to 62;
and/or
CDR3 is selected from the group consisting of:
   g) an amino acid sequence of any one of SEQ ID NOS: 77 to 91;
   h) an amino acid sequence having at least 80% identity with at least one of the amino acid sequences of SEQ ID NOS: 77 to 91;
   i) an amino acid sequence having 3, 2, or 1 amino acid differences with at least one of the amino acid sequences of SEQ ID NOS: 77 to 91;
or any suitable fragment of such an amino acid sequence.

In particular, according to this preferred but non-limiting aspect, the present disclosure relates to a single-domain antibody (as described herein) that immunospecifically binds to SARS-CoV-2, consisting of four framework regions (FR1, FR2, FR3, FR4) and three complementarity determining regions (CDR1, CDR2, CDR3), in which:
CDR1 is selected from the group consisting of:
   a) an amino acid sequence of any one of SEQ ID NOS: 27 to 40;
   b) an amino acid sequence having at least 80% identity with at least one of the amino acid sequences of SEQ ID NOS: 27 to 40;
   c) an amino acid sequence having 3, 2, or 1 amino acid differences with at least one of the amino acid sequences of SEQ ID NOS: 27 to 40;
and
CDR2 is selected from the group consisting of:
   d) an amino acid sequence of any one of SEQ ID NOS: 49 to 62;
   e) an amino acid sequence having at least 80% identity with at least one of the amino acid sequences of SEQ ID NOS: 49 to 62;
   f) an amino acid sequence having 3, 2, or 1 amino acid differences with at least one of the amino acid sequences of SEQ ID NOS: 49 to 62;
and
CDR3 is selected from the group consisting of:
   g) an amino acid sequence of any one of SEQ ID NOS: 77 to 91;
   h) an amino acid sequence having at least 80% identity with at least one of the amino acid sequences of SEQ ID NOS: 77 to 91;
   i) an amino acid sequence having 3, 2, or 1 amino acid differences with at least one of the amino acid sequences of SEQ ID NOS: 77 to 91;
or any suitable fragment of such an amino acid sequence.

As generally described herein, when a single-domain antibody described herein contains one or more CDR1 sequences according to b) and/or c):
i) any amino acid substitution in such a CDR according to b) and/or c) is preferably a conservative amino acid substitution compared to the corresponding CDR according to a); and/or
ii) the CDR according to b) and/or c) preferably only contains amino acid substitutions, and no amino acid deletions or insertions, compared to the corresponding CDR according to a); and/or
iii) the CDR according to b) and/or c) may be a CDR that is derived from a CDR according to a) by means of affinity maturation using one or more known techniques of affinity maturation.

Similarly, when a single-domain antibody described herein contains one or more CDR2 sequences according to e) and/or f):
i) any amino acid substitution in such a CDR according to e) and/or f) is preferably a conservative amino acid substitution compared to the corresponding CDR according to d); and/or
ii) the CDR according to e) and/or f) preferably only contains amino acid substitutions, and no amino acid deletions or insertions, compared to the corresponding CDR according to d); and/or
iii) the CDR according to e) and/or f) may be a CDR that is derived from a CDR according to d) by means of affinity maturation using one or more known techniques of affinity maturation.

Similarly, when a single-domain antibody described herein contains one or more CDR3 sequences according to h) and/or i):
i) any amino acid substitution in such a CDR according to h) and/or i) is preferably a conservative amino acid substitution compared to the corresponding CDR according to g); and/or
ii) the CDR according to h) and/or i) preferably only contains amino acid substitutions, and no amino acid deletions or insertions, compared to the corresponding CDR according to g); and/or
iii) the CDR according to h) and/or i) may be a CDR that is derived from a CDR according to g) by means of affinity maturation using one or more known techniques of affinity maturation.

It should be understood that the last three paragraphs generally apply to any single-domain antibody described herein that comprises one or more CDR1 sequences, CDR2 sequences and/or CDR3 sequences according to b), c), e), f), h) or i).

Single-domain antibodies comprising one or more of the CDRs listed above are preferred; single-domain antibodies comprising two or more of the CDRs listed above are more preferred; and single-domain antibodies comprising three of the CDRs listed above are most preferred.

Some preferred, but non-limiting, combinations of CDR sequences, as well as preferred combinations of CDR sequences and framework sequences, are mentioned in Table 1 below, which lists the CDR sequences and framework sequences that are present in a number of exemplary single-domain antibodies described herein. A combination of CDR1, CDR2 and CDR3 sequences that occur in the same exemplary single-domain antibody (e.g., CDR1, CDR2 and CDR3 sequences that are mentioned on the same line in Table 1) will usually be preferred, although the invention in its broadest sense is not limited thereto, and also comprises other suitable combinations of the CDR sequences mentioned in Table 1. Also, a combination of CDR sequences and framework sequences that occur in the same exemplary single-domain antibody (e.g., CDR sequences and framework sequences that are mentioned on the same line in Table 1) will usually be preferred, although the invention in its broadest sense is not limited thereto, and also comprises other suitable combinations of the CDR sequences and framework sequences mentioned in Table 1, as well as combinations of such CDR sequences and other suitable framework sequences, e.g., as further described herein.

Also, in the single-domain antibodies described herein that comprise the combinations of CDRs mentioned in Table 1, each CDR can be replaced by a CDR selected from the group consisting of an amino acid sequence having at least 80%, at least 90%, at least 95%, or at least 99% sequence identity with the mentioned CDRs; in which:
i) any amino acid substitution in such a CDR is preferably a conservative amino acid substitution compared to the corresponding CDR sequence mentioned in Table 1; and/or
ii) any such CDR sequence preferably only contains amino acid substitutions, and no amino acid deletions or insertions, compared to the corresponding CDR sequence mentioned in Table 1; and/or
iii) any such CDR sequence is a CDR that is derived by means of a known technique for affinity maturation, and in particular starting from the corresponding CDR sequence mentioned in Table 1.

The combinations of CDR sequences, as well as the combinations of CDR sequences and framework sequences mentioned in Table 1 will generally be preferred.

TABLE 1
Framework and CDR Sequences of Exemplary Anti-SARS-COV-2 Single-Domain Antibodies

| ID | FR1 | CDR1 | FR2 | CDR2 | FR3 | CDR3 | FR4 |
|---|---|---|---|---|---|---|---|
| Nb15 | QVQLQESGGGL | LPFSDYLMG | WFRQAPGK | AISQNGGHT | YADSVLGRFTISRD | AARRPGGGRW | YWGQGTQV |
| | VQAGGSLRVSC | (SEQ ID: | EREYVA | (SEQ ID: 49) | NAKNTVYLQMNML | DAAHDYN | TVSS |
| | AASG | 27) | (SEQ ID: | | TPGDTAVYSC | (SEQ ID: 77) | (SEQ ID: 92) |
| | (SEQ ID: 16) | | 41) | | (SEQ ID: 63) | | |
| Nb17 | QVQLQESGGGL | RTFGIYRMG | WFRQAPGK | AITSSADTAQ | YRDSVKGRFAISR | AARDPTTLEYG | NWGQGTQV |
| | VQTGGSLRLSC | (SEQ ID: | EREFVA | (SEQ ID: 50) | DNAKNTLYLQMNS | (SEQ ID: 78) | TVSS |
| | AASG | 28) | (SEQ ID: | | LKPEDTAIYYC | | (SEQ ID: 93) |
| | (SEQ ID: 17) | | 42) | | (SEQ ID: 64) | | |
| Nb19 | QVQLQESGGGL | SGFSIHAMG | WYRQAPGK | VVGHKTN | YADSVKGRFTISRD | YCNTIVTMTGV | VWGQGTQV |
| | VQAGGSLRLSC | (SEQ ID: | QREFVA | (SEQ ID: 51) | VGKNTVELQMNSL | PDA | TVSS |
| | AASG | 29) | (SEQ ID: | | KVEDTAVYYC | (SEQ ID: 79) | (SEQ ID: 94) |
| | (SEQ ID: 18) | | 43) | | (SEQ ID: 65) | | |
| Nb56 | QVQLQESGGGL | RTFRRYGMG | WFRQAPGK | AVDRSHTK | YADFVKGRFTISTN | AAPSYEKGSDP | YWGQGTQV |
| | VQAGDSLRLSC | (SEQ ID: | EREFVA | TG | YENMVYLQMNSLK | TSWNTDRGYD | TVSS |
| | VASE | 30) | (SEQ ID: | (SEQ ID: 52) | PEDTAVYYC | (SEQ ID: 80) | (SEQ ID: 92) |
| | (SEQ ID: 19) | | 42) | | (SEQ ID: 66) | | |
| Nb6 | QVQLQESGGGL | NIANSNSMA | WWRQTPG | IISHGVTN | YADSVKGRFTVSR | YADLFGNT | YWGQGTQV |
| | VQAGGSLRLSC | (SEQ ID: | NQHERVA | (SEQ ID: 53) | DNAKNTLYLQMNN | (SEQ ID: 81) | TVSS |
| | AASG | 31) | (SEQ ID: | | LKPEDTAAYYC | | (SEQ ID: 92) |
| | (SEQ ID: 18) | | 44) | | (SEQ ID: 67) | | |
| Nb7 | QVQLQESGGGL | RTFRRYGIA | WFRQAPGK | AVDRSHS | YADFVQGRFTISTV | AAASYEKGSDY | YWGQGTQV |
| | VQAGDSLRLSC | (SEQ ID: | EREFVA | QTN | YAKNMVYLQMNSL | TSWNTDRGYD | TVSS |
| | LSSE | 32) | (SEQ ID: | (SEQ ID: 54) | KPEDTAVYYC | (SEQ ID: 82) | (SEQ ID: 92) |
| | (SEQ ID: 20) | | 42) | | (SEQ ID: 68) | | |
| Nb43 | QVQLQESGGGL | RTFSDSAMG | WFRQAPGE | VITWNGGT | YADSVKGRFTISRD | AADTSRWDYS | YWGQGTQV |
| | VQAGDSLKLSC | (SEQ ID: | EREFVA | TY | DAKNTVYLQMNSL | LTYHYTREYN | TVSS |
| | AASG | 33) | (SEQ ID: | (SEQ ID: 55) | KPEDTAVYYC | (SEQ ID: 83) | (SEQ ID: 92) |
| | (SEQ ID: 21) | | 45) | | (SEQ ID: 69) | | |
| Nb47 | QVQLQESGGGL | RTLSRYSMS | WFRQAPGK | GIRWSGSN | YADSMKQRFTISQ | AATSRSDHYLN | YWGQGTQV |
| | VQAGGSLRLSC | (SEQ ID: | EREFVA | TY | DNVKNTVHLQMNS | AVAWTLPNEYD | TVSS |
| | AASG | 34) | (SEQ ID: | (SEQ ID: 56) | LKPEDTAVYYC | (SEQ ID: 84) | (SEQ ID: 92) |
| | (SEQ ID: 18) | | 42) | | (SEQ ID: 70) | | |
| Nb50 | QVQLQESGGGL | RTFDGTYRMG | WFRQGPGK | AIGFGVST | YADSVKGRFTISRN | AARPPPYTEYN | YWGQGTQV |
| | VQAGGSLRLSC | (SEQ ID: | EREFVA | TS | NAKNTVYLQMNSL | (SEQ ID: 85) | TVSS |
| | AASG | 35) | (SEQ ID: | (SEQ ID: 57) | KPEDTAVYYC | | (SEQ ID: 92) |
| | (SEQ ID: 8) | | 46) | | (SEQ ID: 71) | | |
| Nb53 | QVQLQESGGGV | LTFSSYLMA | WFRQAPGK | RINWNGRV | TVDSVKGRFIISRD | AADRNYGTGG | YWGQGTQV |
| | VQAGDSLRLSC | (SEQ ID: | EREFVA | PY | NAKNTVWLQMNSL | AETVYE | TVSS |
| | AASG | 36) | (SEQ ID: | (SEQ ID: 58) | KPEDTAVYYC | (SEQ ID: 86) | (SEQ ID: 92) |
| | (SEQ ID: 22) | | 42) | | (SEQ ID: 72) | | |
| Nb55 | QVQLQESGGGL | RAFSDSSAMA | WFRQAPGK | ALNRVNVA | CRDSVSGRFTISR | ASDLILNDCSR | YWGQGTQV |
| | VQAGGSLRLSC | (SEQ ID: | EREFVA | Y | DNGKNTAYLEMNS | NPARYA | TVSS |
| | EASG | 37) | (SEQ ID: | (SEQ ID: 59) | VKPEDTAIYYC | (SEQ ID: 87) | (SEQ ID: 92) |
| | (SEQ ID:23) | | 42) | | (SEQ ID: 73) | | |
| Nb60 | QVQLQESGGGL | RTFRRYG | WFRQAPGK | AIDRSHSN | YADFVKGRFTISTV | AAATYEKGSDP | VWGQGTQV |
| | VQAGDSLRLSC | MG | EREFVA | TD | YAKNMVYLQMNSL | TSWNTDRGYD | TVSS |
| | LASE | (SEQ ID: | (SEQ ID: | (SEQ ID: 60) | KPEDTAVYYC | (SEQ ID: 88) | (SEQ ID: 94) |
| | (SEQ ID: 24) | 30) | 42) | | (SEQ ID: 74) | | |
| msNb12 | QVKLEESGGGS | LTHNNCGLD | WYRRAPGK | SISADGTT | YADSVKGRFTISKD | KTAFPYFGNSC | YWGQGTSV |
| | VQAGGSLRLICT | (SEQ ID: | EREFVS | S | KVEDTVYLQMNSL | VLD | TVSS |
| | APG | 38) | (SEQ ID: | (SEQ ID: 61) | KPEDTAIYSC | (SEQ ID: 89) | (SEQ ID: 95) |
| | (SEQ ID: 25) | | 47) | | (SEQ ID: 75) | | |
| mNb30 | QVQLVESGGGL | LTFSKYAMG | WFRQAPGK | TISWSGDS | YADSVKGRFTISRD | AADRGMGYGD | YWGQGTSV |
| | VQAGGSLRLSC | (SEQ ID: | ERKFVA | AF | NARNTVYLQMNSL | FMD | TASS |
| | AASG | 39) | (SEQ ID: | (SEQ ID: 62) | KPEDTAVYYC | (SEQ ID: 90) | (SEQ ID: 96) |
| | (SEQ ID: 26) | | 48) | | (SEQ ID: 76) | | |
| mNB35 | QVQLVESGGGL | RTFSKYAMG | WFRQAPGK | TISWSGDS | YADSVKGRFTISRD | TADRGMGYGD | YWGQGTSV |
| | VQAGGSLRLSC | (SEQ ID: | ERKFVA | AF | NARNTVYLQMNSL | FMD | TVSS |
| | AASG | 40) | (SEQ ID: | (SEQ ID: 62) | KPEDTAVYYC | (SEQ ID: 91) | (SEQ ID: 95) |
| | (SEQ ID: 26) | | 48) | | (SEQ ID: 76) | | |

Provided herein are exemplary single-domain antibodies that immunospecifically bind to SARS-CoV-2 with high affinity and neutralize SARS-CoV-2 and SARS-CoV-2 variants. These single-domain antibodies have been demonstrated, for example, to block SARS-CoV-2 infection and SARS-CoV-2 variant infection of cells in culture.

The amino acid and nucleotide sequences of these exemplary single-domain antibodies are shown below in Tables 2 and 3, respectively.

TABLE 2
Amino Acid Sequences of Exemplary Anti-SARS-COV-2 Single-Domain
Antibodies

| | SEQ | |
|---|---|---|
| | ID | |
| ID | NO | Amino Acid Sequence |
| Nb15 | 1 | QVQLQESGGGLVQAGGSLRVSCAASGLPFSDYLMGWFRQAPGKEREYVA |
| | | AISQNGGHTYADSVLGRFTISRDNAKNTVYLQMNMLTPGDTAVYSCAARR |
| | | PGGGRWDAAHDYNYWGQGTQVTVSS |
| Nb17 | 2 | QVQLQESGGGLVQTGGSLRLSCAASGRTFGIYRMGWFRQAPGKEREFVA |
| | | AITSSADTAQYRDSVKGRFAISRDNAKNTLYLQMNSLKPEDTAIYYCAARDP |
| | | TTLEYGNWGQGTQVTVSS |
| Nb19 | 3 | QVQLQESGGGLVQAGGSLRLSCAASGSGFSIHAMGWYRQAPGKQREFVA |
| | | VVGHKTNYADSVKGRFTISRDVGKNTVELQMNSLKVEDTAVYYCYCNTIVT |
| | | MTGVPDAVWGQGTQVTVSS |
| Nb56 | 4 | QVQLQESGGGLVQAGDSLRLSCVASERTFRRYGMGWFRQAPGKEREFV |
| | | AAVDRSHTKTGYADFVKGRFTISTNYENMVYLQMNSLKPEDTAVYYCAAP |
| | | SYEKGSDPTSWNTDRGYDYWGQGTQVTVSS |
| Nb6 | 5 | QVQLQESGGGLVQAGGSLRLSCAASGNIANSNSMAWWRQTPGNQHERV |
| | | AIISHGVTNYADSVKGRFTVSRDNAKNTLYLQMNNLKPEDTAAYYCYADLF |
| | | GNTYWGQGTQVTVSS |
| Nb7 | 6 | QVQLQESGGGLVQAGDSLRLSCLSSERTFRRYGIAWFRQAPGKEREFVAA |
| | | VDRSHSQTNYADFVQGRFTISTVYAKNMVYLQMNSLKPEDTAVYYCAAAS |
| | | YEKGSDYTSWNTDRGYDYWGQGTQVTVSS |
| Nb43 | 7 | QVQLQESGGGLVQAGDSLKLSCAASGRTFSDSAMGWFRQAPGEEREFVA |
| | | VITWNGGTTYYADSVKGRFTISRDDAKNTVYLQMNSLKPEDTAVYYCAADT |
| | | SRWDYSLTYHYTREYNYWGQGTQVTVSS |
| Nb47 | 8 | QVQLQESGGGLVQAGGSLRLSCAASGRTLSRYSMSWFRQAPGKEREFVA |
| | | GIRWSGSNTYYADSMKQRFTISQDNVKNTVHLQMNSLKPEDTAVYYCAAT |
| | | SRSDHYLNAVAWTLPNEYDYWGQGTQVTVSS |
| Nb50 | 9 | QVQLQESGGGLVQAGGSLRLSCAASGRTFDGTYRMGWFRQGPGKEREF |
| | | VAAIGFGVSTTSYADSVKGRFTISRNNAKNTVYLQMNSLKPEDTAVYYCAA |
| | | RPPPYTEYNYWGQGTQVTVSS |
| Nb53 | 10 | QVQLQESGGGVVQAGDSLRLSCAASGLTFSSYLMAWFRQAPGKEREFVA |
| | | RINWNGRVPYTVDSVKGRFIISRDNAKNTVWLQMNSLKPEDTAVYYCAAD |
| | | RNYGTGGAETVYEYWGQGTQVTVSS |
| Nb55 | 11 | QVQLQESGGGLVQAGGSLRLSCEASGRAFSDSSAMAWFRQAPGKEREFV |
| | | AALNRVNVAYCRDSVSGRFTISRDNGKNTAYLEMNSVKPEDTAIYYCASDLI |
| | | LNDCSRNPARYAYWGQGTQVTVSS |
| Nb60 | 12 | QVQLQESGGGLVQAGDSLRLSCLASERTFRRYGMGWFRQAPGKEREFVA |
| | | AIDRSHSNTDYADFVKGRFTISTVYAKNMVYLQMNSLKPEDTAVYYCAAAT |
| | | YEKGSDPTSWNTDRGYDVWGQGTQVTVSS |
| msNb12 | 13 | QVKLEESGGGSVQAGGSLRLICTAPGLTHNNCGLDWYRRAPGKEREFVSS |
| | | ISADGTTSYADSVKGRFTISKDKVEDTVYLQMNSLKPEDTAIYSCKTAFPYF |
| | | GNSCVLDYWGQGTSVTVSS |
| mNb30 | 14 | QVQLVESGGGLVQAGGSLRLSCAASGLTFSKYAMGWFRQAPGKERKFVA |
| | | TISWSGDSAFYADSVKGRFTISRDNARNTVYLQMNSLKPEDTAVYYCAADR |
| | | GMGYGDFMDYWGQGTSVTASS |
| mNb35 | 15 | QVQLVESGGGLVQAGGSLRLSCAASGRTFSKYAMGWFRQAPGKERKFVA |
| | | TISWSGDSAFYADSVKGRFTISRDNARNTVYLQMNSLKPEDTAVYYCTADR |
| | | GMGYGDFMDYWGQGTSVTVSS |

TABLE 3
Nucleotide Sequences Encoding Exemplary Anti-SARS-COV-2 Single-
Domain Antibodies

| | SEQ | |
|---|---|---|
| | ID | |
| ID | NO | Nucleotide Sequence |
| Nb15 | 97 | CAGGTGCAGCTGCAGGAGTCTGGGGGAGGGTTGGTGCAGGCTGGGGGC |
| | | TCTCTGAGAGTCTCCTGTGCAGCGTCTGGACTTCCCTTCAGTGACTATTT |
| | | AATGGGCTGGTTCCGCCAGGCGCCAGGGAAGGAGCGTGAGTATGTAGC |
| | | CGCTATTAGTCAGAATGGTGGACACACTTATGCAGACTCCGTGCTGGGCC |
| | | GATTCACCATCTCCAGAGACAACGCCAAGAATACGGTGTATCTGCAAATG |
| | | AACATGTTGACACCTGGGGACACGGCCGTTTATAGTTGTGCTGCCCGAA |
| | | GGCCCGGTGGGGGTAGGTGGGATGCCGCCCATGACTATAACTACTGGG |
| | | GCCAGGGGACCCAGGTCACCGTCTCCTCA |
| Nb17 | 98 | CAGGTGCAGCTGCAGGAGTCTGGGGGAGGATTGGTGCAAACTGGGGGC |
| | | TCTCTGAGACTCTCCTGTGCAGCCTCTGGACGCACCTTCGGTATCTATCG |
| | | CATGGGCTGGTTCCGCCAGGCTCCAGGGAAGGAGCGTGAGTTTGTAGCA |
| | | GCTATCACTTCGAGTGCTGATACCGCACAGTATCGAGACTCCGTGAAGG |
| | | GCCGATTCGCCATCTCCAGAGACAACGCCAAGAACACGCTGTATCTGCAA |
| | | ATGAACAGCCTGAAACCTGAGGACACGGCCATTTATTATTGTGCAGCACG |
| | | GGATCCCACTACATTGGAGTATGGCAACTGGGGCCAGGGGACCCAGGTC |
| | | ACCGTCTCCTCA |
| Nb19 | 99 | CAGGTGCAGCTGCAGGAGTCTGGGGGAGGCTTGGTGCAGGCTGGGGGG |
| | | TCTCTGAGACTGTCCTGTGCAGCCTCTGGAAGCGGCTTTAGTATTCATGC |
| | | CATGGGCTGGTACCGCCAGGCTCCAGGGAAGCAGCGCGAGTTCGTCGC |
| | | TGTCGTTGGCCATAAGACGAACTATGCAGACTCCGTTAAGGGCCGATTCA |
| | | CCATCTCCAGAGACGTTGGCAAGAACACGGTGGAGCTGCAAATGAACAG |
| | | CCTGAAAGTTGAGGACACAGCCGTCTATTATTGTTACTGCAATACTATCGT |
| | | GACTATGACAGGGGTTCCTGATGCCGTCTGGGGCCAGGGGACCCAGGT |
| | | CACCGTCTCCTCA |
| Nb56 | 100 | CAGGTGCAGCTGCAGGAGTCTGGGGGAGGATTGGTGCAGGCTGGGGAC |
| | | TCTCTGAGACTCTCCTGTGTAGCCTCTGAACGCACATTCAGGCGCTATGG |
| | | CATGGGCTGGTTCCGCCAGGCTCCAGGGAAGGAGCGTGAGTTTGTAGCA |
| | | GCTGTTGACCGGAGTCATACTAAGACAGGCTATGCAGACTTCGTGAAGG |
| | | GCCGATTCACCATCTCCACGAACTACGAGAACATGGTGTATCTGCAAATG |
| | | AACAGCCTGAAACCTGAGGACACGGCCGTTTATTACTGTGCTGCGCCGT |
| | | CGTACGAGAAAGGGTCGGACCCTACTAGTTGGAACACCGACAGAGGGTA |
| | | TGACTACTGGGGCCAGGGGACCCAGGTCACCGTCTCCTCA |
| Nb6 | 101 | CAGGTGCAGCTGCAGGAGTCTGGGGGAGGCTTGGTGCAGGCTGGGGGG |
| | | TCTCTGAGACTCTCCTGTGCAGCCTCTGGAAACATCGCGAATTCCAATTC |
| | | CATGGCCTGGTGGCGCCAGACTCCAGGAAACCAGCACGAGCGGGTCGC |
| | | CATTATTAGTCATGGTGTAACAAACTATGCAGATTCCGTGAAGGGCCGAT |
| | | TCACAGTGTCCAGAGACAACGCCAAGAATACTTTGTATCTGCAAATGAAC |
| | | AACCTGAAACCTGAGGACACAGCCGCCTATTATTGTTATGCAGATCTCTT |
| | | CGGAAACACCTACTGGGGCCAGGGGACCCAGGTCACCGTCTCCTCA |
| Nb7 | 102 | CAGGTGCAGCTGCAGGAGTCTGGAGGAGGATTGGTGCAGGCTGGGGAC |
| | | TCTCTGAGACTCTCCTGTCTATCCTCTGAACGCACATTCAGGCGCTATGG |
| | | CATAGCCTGGTTCCGCCAGGCTCCAGGGAAGGAGCGTGAGTTTGTAGCA |
| | | GCTGTTGACCGGAGTCATAGTCAGACAAACTATGCAGACTTCGTACAGGG |
| | | CCGATTCACCATCTCCACGGTCTACGCCAAGAACATGGTGTATCTGCAAA |
| | | TGAACAGCCTGAAACCTGAGGACACGGCCGTTTATTACTGTGCTGCGGC |
| | | GTCGTACGAGAAAGGGTCGGACTATACTAGTTGGAACACCGACAGAGGG |
| | | TATGACTACTGGGGCCAGGGGACCCAGGTCACCGTCTCCTCA |
| Nb43 | 103 | CAGGTGCAGCTGCAGGAGTCTGGGGGAGGATTGGTGCAGGCTGGGGAC |
| | | TCTCTGAAACTCTCCTGTGCAGCCTCTGGACGCACCTTCAGTGACAGTGC |
| | | CATGGGCTGGTTCCGCCAGGCTCCAGGAGAGGAGCGTGAGTTTGTAGCA |
| | | GTTATTACCTGGAATGGTGGCACCACATACTATGCAGACTCCGTGAAGGG |
| | | CCGATTCACCATCTCCAGAGACGACGCCAAGAACACGGTGTACCTGCAA |
| | | ATGAACAGCCTGAAACCTGAGGACACGGCCGTTTATTACTGTGCAGCGG |
| | | ACACAAGCCGGTGGGACTATAGTCTTACATACCACTACACGAGGGAGTAT |
| | | AACTACTGGGGCCAGGGGACCCAGGTCACCGTCTCCTCA |
| Nb47 | 104 | CAGGTGCAGCTGCAGGAGTCTGGGGGAGGATTGGTGCAGGCTGGGGGC |
| | | TCTCTGAGACTCTCCTGTGCAGCCTCTGGACGCACCTTGAGTAGGTATTC |
| | | CATGAGCTGGTTCCGCCAGGCTCCAGGGAAGGAGCGTGAGTTTGTAGCA |
| | | GGTATACGGTGGAGTGGTAGTAACACATACTATGCAGACTCCATGAAGCA |
| | | GCGATTCACCATCTCCCAAGACAATGTCAAGAACACGGTGCATCTGCAAA |
| | | TGAACAGCCTGAAACCTGAGGACACGGCCGTTTATTACTGTGCAGCCACA |
| | | AGTAGAAGTGATCATTACTTGAATGCCGTGGCTTGGACCCTTCCGAATGA |
| | | GTATGACTACTGGGGCCAGGGGACCCAGGTCACCGTCTCCTCA |
| Nb50 | 105 | CAGGTGCAGCTGCAGGAGTCTGGGGGAGGATTGGTGCAGGCTGGGGGA |
| | | TCTCTGAGACTCTCCTGTGCAGCCTCTGGACGCACCTTCGATGGTACCTA |
| | | TCGCATGGGCTGGTTCCGCCAGGGTCCAGGGAAGGAGCGTGAGTTTGTA |
| | | GCAGCTATAGGCTTCGGTGTTAGTACCACATCGTATGCAGACTCCGTGAA |
| | | GGGCCGATTCACCATCTCCAGAAACAACGCCAAGAACACGGTGTATCTG |
| | | CAAATGAACAGCCTGAAACCTGAGGACACGGCCGTTTATTACTGCGCAGC |
| | | GCGCCCCCCGCCTTACACGGAGTATAACTACTGGGGCCAGGGGACCCA |
| | | GGTCACCGTCTCCTCA |
| Nb53 | 106 | CAGGTGCAGCTGCAGGAGTCTGGAGGAGGAGTGGTGCAGGCTGGGGAC |
| | | TCTCTGAGACTCTCCTGTGCAGCCTCTGGACTCACCTTCAGTAGTTATCT |
| | | CATGGCCTGGTTCCGCCAGGCTCCAGGGAAGGAGCGTGAGTTTGTAGCA |
| | | CGTATTAACTGGAATGGTCGTGTGCCATACACTGTAGACTCTGTGAAGGG |
| | | CCGATTCATCATCTCCAGAGACAATGCCAAAAACACGGTGTGGCTGCAAA |
| | | TGAACAGCCTGAAACCTGAGGACACGGCCGTTTATTACTGTGCAGCAGAC |
| | | CGGAACTACGGCACAGGGGGCGCCGAAACAGTGTATGAGTACTGGGGC |
| | | CAGGGGACCCAGGTCACCGTCTCCTCA |
| Nb55 | 107 | CAGGTGCAGCTGCAGGAGTCTGGGGGAGGATTGGTGCAGGCTGGGGGC |
| | | TCTCTGAGACTCTCCTGTGAAGCCTCTGGACGCGCCTTCAGTGACTCGTC |
| | | GGCCATGGCCTGGTTCCGCCAGGCTCCAGGGAAGGAGCGTGAGTTTGTA |
| | | GCGGCGCTTAACAGAGTTAATGTTGCATATTGTAGAGACTCCGTGTCGGG |
| | | CCGATTCACCATCTCCAGAGACAACGGCAAGAATACGGCATATCTGGAAA |
| | | TGAACAGTGTGAAACCTGAGGACACGGCCATTTATTACTGTGCATCAGAT |
| | | CTAATCCTAAATGATTGCAGTCGAAACCCCGCGAGGTATGCCTACTGGGG |
| | | CCAGGGGACCCAGGTCACCGTCTCCTCA |
| Nb60 | 108 | CAGGTGCAGCTGCAGGAGTCTGGGGGAGGATTGGTGCAGGCTGGGGAC |
| | | TCTCTGAGACTCTCCTGTCTAGCCTCTGAACGCACATTCAGGCGCTATGG |
| | | CATGGGCTGGTTCCGCCAGGCTCCAGGGAAGGAGCGTGAGTTTGTAGCA |
| | | GCTATTGACCGGAGTCATAGTAATACAGACTATGCAGACTTCGTGAAGGG |
| | | CCGATTCACCATCTCCACGGTCTACGCCAAGAACATGGTGTATCTGCAAA |
| | | TGAACAGCCTGAAACCTGAGGACACGGCCGTTTATTACTGTGCTGCGGC |
| | | GACGTACGAGAAAGGGTCGGACCCTACTAGTTGGAACACCGACAGAGGG |
| | | TATGACGTCTGGGGCCAGGGGACCCAGGTCACCGTCTCCTCA |
| msNb12 | 109 | CAAGTAAAACTTGAAGAATCAGGTGGTGGAAGCGTTCAAGCTGGTGGATC |
| | | TCTCAGACTTATTTGTACCGCACCCGGCTTAACACATAATAACTGTGGCTT |
| | | GGACTGGTACAGACGAGCACCAGGCAAGGAACGCGAGTTCGTTTCATCA |
| | | ATAAGTGCAGATGGAACCACTTCATACGCAGATTCCGTCAAGGGACGGTT |
| | | TACCATTAGTAAGGACAAAGTCGAGGACACAGTCTACCTCCAAATGAACA |
| | | GTTTGAAGCCAGAAGACACTGCTATTTATTCATGCAAGACAGCCTTCCCTT |
| | | ACTTCGGTAATAGCTGTGTTTTGGACTACTGGGGTCAAGGAACCTCAGTC |
| | | ACCGTCTCCTCG |
| mNb30 | 110 | CAGGTGCAATTGGTAGAGTCTGGCGGTGGACTCGTTCAAGCCGGAGGCT |
| | | CACTGCGGTTGTCTTGTGCCGCATCTGGTCTAACCTTCTCAAAATATGCTA |
| | | TGGGGTGGTTCCGGCAGGCTCCAGGTAAAGAGCGGAAATTTGTCGCAAC |
| | | AATTAGTTGGTCTGGTGATAGTGCCTTCTATGCTGATTCAGTAAAAGGTCG |
| | | ATTCACTATATCACGGGATAACGCAAGAAATACTGTCTATCTCCAAATGAA |
| | | CTCTCTGAAGCCTGAAGATACTGCTGTGTATTACTGTGCCGCAGATCGAG |
| | | GAATGGGGTATGGGGATTTTATGGACTACTGGGGTCAAGGAACCTCAGT |
| | | CACCGCCTCCTCGGCCTCAGGGGCC |
| mNb35 | 111 | CAGGTCCAACTGGTAGAGTCAGGCGGTGGACTCGTTCAAGCCGGAGGCT |
| | | CACTGCGGTTGTCTTGTGCCGCATCTGGTCGAACCTTCTCAAAATATGCT |
| | | ATGGGGTGGTTCCGGCAGGCTCCAGGTAAAGAGCGGAAATTTGTCGCAA |
| | | CAATTAGTTGGTCTGGTGATAGTGCCTTCTATGCTGATTCAGTAAAAGGTC |
| | | GATTCACTATATCACGGGATAACGCAAGAAATACTGTCTATCTCCAAATGA |
| | | ACTCTCTGAAGCCTGAAGATACTGCTGTGTATTACTGTACCGCAGATCGA |
| | | GGAATGGGGTATGGGGATTTTATGGACTACTGGGGTCAAGGTACCTCAG |
| | | TCACCGTCTCCTCGGCCTCAGGGGCC |

In an embodiment, at least one of the CDR1, CDR2 and CDR3 sequences present in the single-domain antibodies described herein is selected from the group consisting of the CDR1, CDR2 and CDR3 sequences, respectively, listed in Table 1; or from the group of CDR1, CDR2 and CDR3 sequences, respectively, that have at least 80%, preferably at least 90%, more preferably at least 95%, even more preferably at least 99% identity with at least one of the CDR1, CDR2 and CDR3 sequences, respectively, listed in Table 1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table 1.

CDR sequences are preferably selected such that the single-domain antibodies described herein bind to SARS-CoV-2 with an affinity (measured and/or expressed as a K_{D} value (actual or apparent), a K_{A} value (actual or apparent), a kₒₙ rate and/or a k_{off} rate, or alternatively as an IC₅₀ value) as described herein.

In an embodiment, at least the CDR3 sequence present in the single-domain antibodies described herein is selected from the group consisting of the CDR3 sequences listed in Table 1 or from the group of CDR3 sequences that have at least 80%, at least 90%, at least 95%, or at least 99% identity with at least one of the CDR3 sequences listed in Table 1; and/or from the group consisting of the CDR3 sequences that have 3, 2 or only 1 amino acid difference(s) with at least one of the CDR3 sequences listed in Table 1.

Preferably, at least two of the CDR1, CDR2 and CDR3 sequences present in the single-domain antibodies described herein are selected from the group consisting of the CDR1, CDR2 and CDR3 sequences, respectively, listed in Table 1 or from the group consisting of CDR1, CDR2 and CDR3 sequences, respectively, that have at least 80%, at least 90%, at least 95%, or at least 99% identity with at least one of the CDR1, CDR2 and CDR3 sequences, respectively, listed in Table 1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table 1.

In particular, at least the CDR3 sequence present in the single-domain antibodies described herein is selected from the group consisting of the CDR3 sequences listed in Table 1 or from the group of CDR3 sequences that have at least 80%, at least 90%, at least 95%, or at least 99% sequence identity with at least one of the CDR3 sequences listed in Table 1, respectively; and at least one of the CDR1 and CDR2 sequences present is selected from the group consisting of the CDR1 and CDR2 sequences, respectively, listed in Table 1 or from the group of CDR1 and CDR2 sequences, respectively, that have at least 80%, at least 90%, at least 95%, or at least 99% identity with at least one of the CDR1 and CDR2 sequences, respectively, listed in Table 1; and/or from the group consisting of the CDR1 and CDR2 sequences, respectively, that have 3, 2 or only 1 amino acid difference(s) with at least one of the CDR1 and CDR2 sequences, respectively, listed in Table 1.

Preferably, all three CDR1, CDR2 and CDR3 sequences present in the single-domain antibodies described herein are selected from the group consisting of the CDR1, CDR2 and CDR3 sequences, respectively, listed in Table 1 or from the group of CDR1, CDR2 and CDR3 sequences, respectively, that have at least 80%, at least 90%, at least 95%, or at least 99% identity with at least one of the CDR1, CDR2 and CDR3 sequences, respectively, listed in Table 1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table 1.

More preferably, at least one of the CDR1, CDR2 and CDR3 sequences present in the single-domain antibodies described herein is selected from the group consisting of the CDR1, CDR2 and CDR3 sequences, respectively, listed in Table 1. Preferably, in this aspect, at least one or preferably both of the other two CDR sequences present are selected from CDR sequences that have at least 80%, at least 90%, at least 95%, or at least 99% identity with at least one of the corresponding CDR sequences, respectively, listed in Table 1; and/or from the group consisting of the CDR sequences that have 3, 2 or only 1 amino acid difference(s) with at least one of the corresponding sequences, respectively, listed in Table 1.

In particular, at least the CDR3 sequence present in the single-domain antibodies described herein is selected from the group consisting of the CDR3 sequences listed in Table 1. Preferably, in this aspect, at least one and preferably both of the CDR1 and CDR2 sequences present are selected from the groups of CDR1 and CDR2 sequences, respectively, that have at least 80%, at least 90%, at least 95%, or at least 99% identity with the CDR1 and CDR2 sequences, respectively, listed in Table 1; and/or from the group consisting of the CDR1 and CDR2 sequences, respectively, that have 3, 2 or only 1 amino acid difference(s) with at least one of the CDR1 and CDR2 sequences, respectively, listed in Table 1.

More preferably, at least two of the CDR1, CDR2 and CDR3 sequences present in the single-domain antibodies described herein are selected from the group consisting of the CDR1, CDR2 and CDR3 sequences, respectively, listed in Table 1. Preferably, in this aspect, the remaining CDR sequence present is selected from the group of CDR sequences that have at least 80%, at least 90%, at least 95%, or at least 99% identity with at least one of the corresponding CDR sequences listed in Table 1; and/or from the group consisting of CDR sequences that have 3, 2 or only 1 amino acid difference(s) with at least one of the corresponding sequences listed in Table 1.

In particular, at least the CDR3 sequence present in the single-domain antibodies described herein is selected from the group consisting of the CDR3 sequences listed in Table 1, and either the CDR1 sequence or the CDR2 sequence is selected from the group consisting of the CDR1 and CDR2 sequences, respectively, listed in Table 1. Preferably, in this aspect, the remaining CDR sequence present is selected from the group of CDR sequences that have at least 80%, at least 90%, at least 95%, or at least 99% identity with at least one of the corresponding CDR sequences listed in Table 1; and/or from the group consisting of CDR sequences that have 3, 2 or only 1 amino acid difference(s) with the corresponding CDR sequences listed in Table 1.

Preferably, all three CDR1, CDR2 and CDR3 sequences present in the single-domain antibodies described herein are selected from the group consisting of the CDR1, CDR2 and CDR3 sequences, respectively, listed in Table 1.

Also, generally, the combinations of CDRs listed in Table 1 (e.g., those mentioned within the same row in Table 1) are preferred. Thus, it is generally preferred that, when a CDR in a single-domain antibody described herein is a CDR sequence mentioned in Table 1 or is selected from the group of CDR sequences that have at least 80%, at least 90%, at least 95%, or at least 99% identity with a CDR sequence listed in Table 1; and/or from the group consisting of CDR sequences that have 3, 2 or only 1 amino acid difference(s) with a CDR sequence listed in Table 1, that at least one and preferably both of the other CDRs are selected from the CDR sequences that belong to the same exemplary single-domain antibody sequence in Table 1 (e.g., mentioned within the same row in Table 1) or are selected from the group of CDR sequences that have at least 80%, at least 90%, at least 95%, or at least 99% identity with the CDR sequence(s) belonging to the same exemplary single-domain antibody sequence and/or from the group consisting of CDR sequences that have 3, 2 or only 1 amino acid difference(s) with the CDR sequence(s) belonging to the same exemplary single-domain antibody sequence. The other preferences indicated in the above paragraphs also apply to the combinations of CDRs mentioned in Table 1.

A single-domain antibody described herein may comprise a CDR1 sequence having more than 80% identity with any one of the CDR1 sequences mentioned in Table 1, a CDR2 sequence having 3, 2 or 1 amino acid difference(s) with any one of the CDR2 sequences mentioned in Table 1, and a CDR3 sequence (mentioned or not mentioned in Table 1).

Single-domain antibodies described herein may, for example, comprise: (1) a CDR1 sequence having more than 80% identity with any one of the CDR1 sequences mentioned in Table 1; a CDR2 sequence having 3, 2 or 1 amino acid difference(s) with any one of the CDR2 sequences mentioned in Table 1; and a CDR3 sequence having more than 80% identity with any one of the CDR3 sequences mentioned in Table 1; or (2) a CDR1 sequence having more than 80% identity with any one of the CDR1 sequences mentioned in Table 1; a CDR2 sequence, and any one of the CDR3 sequences mentioned in Table 1; or (3) a CDR1 sequence; a CDR2 sequence having more than 80% identity with any one of the CDR2 sequences mentioned in Table 1; and a CDR3 sequence having 3, 2 or 1 amino acid difference(s) with the CDR3 sequence mentioned in Table 1 that belongs to the same exemplary single-domain antibody sequence as the CDR2 sequence.

Other single-domain antibodies described herein may, for example, comprise: (1) a CDR1 sequence having more than 80% identity with any one of the CDR1 sequences mentioned in Table 1; a CDR2 sequence that has 3, 2 or 1 amino acid difference(s) with the CDR2 sequence mentioned in Table 1 that belongs to the same exemplary single-domain antibody sequence; and a CDR3 sequence having more than 80% identity with the CDR3 sequence mentioned in Table 1 that belongs to the same exemplary single-domain antibody sequence; (2) a CDR1 sequence; a CDR2 sequence mentioned in Table 1 and a CDR3 sequence mentioned in Table 1 (in which the CDR2 sequence and CDR3 sequence may belong to different exemplary single-domain antibody sequences).

Other single-domain antibodies described herein may, for example, comprise: (1) a CDR1 sequence having more than 80% identity with any one of the CDR1 sequences mentioned in Table 1; the CDR2 sequence mentioned in Table 1 that belongs to the same exemplary single-domain antibody; and a CDR3 sequence mentioned in Table 1 that belongs to a different exemplary single-domain antibody; or (2) a CDR1 sequence mentioned in Table 1; a CDR2 sequence having 3, 2 or 1 amino acid difference(s) with the CDR2 sequence mentioned in Table 1 that belongs to the same exemplary single-domain antibody; and a CDR3 sequence having more than 80% identity with any one of the CDR3 sequence mentioned in Table 1.

Preferred single-domain antibodies described herein may, for example, comprise a CDR1 sequence mentioned in Table 1, a CDR2 sequence having more than 80% identity with the CDR2 sequence mentioned in Table 1 that belongs to the same exemplary single-domain antibody; and the CDR3 sequence mentioned in Table 1 that belongs to the same exemplary single-domain antibody.

Most preferably, single-domain antibodies described herein comprise CDR1, CDR2, and CDR3 sequences which are selected from one of the combinations of CDR1, CDR2, and CDR3 sequences, respectively, listed in Table 1.

In an embodiment, (a) CDR1 has a length of between 1 and 15 amino acid residues, and usually between 4 and 12 amino acid residues, such as 9 or 10 amino acid residues; and/or (b) CDR2 has a length of between 1 and 15 amino acid residues, and usually between 2 and 12 amino acid residues, such as 7, 8, 9, or 10 amino acid residues; and/or (c) CDR3 has a length of between 2 and 35 amino acid residues, and usually between 3 and 30 amino acid residues, such as between 8 and 22 amino acid residues.

Generally, single-domain antibodies with the above CDR sequences may be as further described herein, and preferably have framework sequences that are also as further described herein.

In an embodiment, the present disclosure relates to a single-domain antibody that immunospecifically binds to SARS-CoV-2, consisting of four framework regions (FR1, FR2, FR3, FR4) and three complementarity determining regions (CDR1, CDR2 CDR3), in which:
FR1 is selected from the group consisting of:
   a) an amino acid sequence of any one of SEQ ID NOS: 16 to 26;
   b) an amino acid sequence having at least 80% identity with at least one of the amino acid sequences of SEQ ID NOS: 16 to 26;
   c) an amino acid sequence having 5, 4, 3, 2, or 1 amino acid differences with at least one of the amino acid sequences of SEQ ID NOS: 16 to 26;
and/or
FR2 is selected from the group consisting of:
   d) an amino acid sequence of any one of SEQ ID NOS: 41 to 48;
   e) an amino acid sequence having at least 80% identity with at least one of the amino acid sequences of SEQ ID NOS: 41 to 48;
   f) an amino acid sequence having 5, 4, 3, 2, or 1 amino acid differences with at least one of the amino acid sequences of SEQ ID NOS: 41 to 48;
and/or
FR3 is selected from the group consisting of:
   g) an amino acid sequence of any one of SEQ ID NOS: 63 to 76;
   h) an amino acid sequence having at least 80% identity with at least one of the amino acid sequences of SEQ ID NOS: 63 to 76;
   i) an amino acid sequence having 5, 4, 3, 2, or 1 amino acid differences with at least one of the amino acid sequences of SEQ ID NOS: 63 to 76;
and/or
FR4 is selected from the group consisting of:
   j) an amino acid sequence of any one of SEQ ID NOS: 92 to 96;
   k) an amino acid sequence having at least 80% identity with at least one of the amino acid sequences of SEQ ID NOS: 92 to 96;
   l) an amino acid sequence having 5, 4, 3, 2, or 1 amino acid differences with at least one of the amino acid sequences of SEQ ID NOS: 92 to 96;
or any suitable fragment of such an amino acid sequence.

A single-domain antibody that immunospecifically binds to SARS-CoV-2 may consist of four framework regions (FR1, FR2, FR3, FR4) and three complementarity determining regions (CDR1, CDR2, CDR3), in which:
FR1 is selected from the group consisting of:
   a) an amino acid sequence of any one of SEQ ID NOS: 16 to 26;
   b) an amino acid sequence having at least 80% identity with at least one of the amino acid sequences of SEQ ID NOS: 16 to 26;
   c) an amino acid sequence having 5, 4, 3, 2, or 1 amino acid differences with at least one of the amino acid sequences of SEQ ID NOS: 16 to 26;
and
FR2 is selected from the group consisting of:
   d) an amino acid sequence of any one of SEQ ID NOS: 41 to 48;
   e) an amino acid sequence having at least 80% identity with at least one of the amino acid sequences of SEQ ID NOS: 41 to 48;
   f) an amino acid sequence having 5, 4, 3, 2, or 1 amino acid differences with at least one of the amino acid sequences of SEQ ID NOS: 41 to 48;
and
FR3 is selected from the group consisting of:
   g) an amino acid sequence of any one of SEQ ID NOS: 63 to 76;
   h) an amino acid sequence having at least 80% identity with at least one of the amino acid sequences of SEQ ID NOS: 63 to 76;
   i) an amino acid sequence having 5, 4, 3, 2, or 1 amino acid differences with at least one of the amino acid sequences of SEQ ID NOS: 63 to 76;
and
FR4 is selected from the group consisting of:
   j) an amino acid sequence of any one of SEQ ID NOS: 92 to 96;
   k) an amino acid sequence having at least 80% identity with at least one of the amino acid sequences of SEQ ID NOS: 92 to 96;
   l) an amino acid sequence having 5, 4, 3, 2, or 1 amino acid differences with at least one of the amino acid sequences of SEQ ID NOS: 92 to 96;
or any suitable fragment of such an amino acid sequence.

Single-domain antibodies comprising one or more of the FRs listed above are preferred; single-domain antibodies comprising two or more of the FRs listed above are more preferred; single-domain antibodies comprising three or more of the FRs listed above are more preferred; and single-domain antibodies comprising four of the FRs listed above are most preferred.

Combinations of FR sequences, as well as preferred combinations of CDR sequences and FR sequences, are mentioned in Table 1 above, which lists the CDR sequences and FR sequences that are present in a number of exemplary single-domain antibodies described herein. A combination of FR1, FR2, FR3, and FR4 sequences that occur in the same exemplary single-domain antibody (e.g., FR1, FR2, FR3, and FR4 sequences that are mentioned on the same line in Table 1) may be preferred, although the invention in its broadest sense is not limited thereto, and also comprises other suitable combinations of the FR sequences mentioned in Table 1. Also, a combination of CDR sequences and FR sequences that occur in the same exemplary single-domain antibody (e.g., CDR sequences and FR sequences that are mentioned on the same line in Table 1) will usually be preferred, although the invention in its broadest sense is not limited thereto, and also comprises other suitable combinations of the CDR sequences and FR sequences mentioned in Table 1, as well as combinations of such CDR sequences and other suitable FR sequences, e.g., as further described herein.

Also, in the single-domain antibodies described herein that comprise the combinations of FRs mentioned in Table 1, each FR can be replaced by a FR selected from the group consisting of an amino acid sequence having at least 80%, at least 90%, at least 95%, or at least 99% sequence identity with the mentioned FRs.

The combinations of FR sequences, as well as the combinations of CDR sequences and FR sequences mentioned in Table 1 may be preferred.

The single-domain antibodies described herein may be naturally-occurring single-domain antibodies (from any suitable species); naturally-occurring VHH sequences (e.g., from a suitable species of Camelid); single-domain antibodies produced by and/or derived from transgenic animals (e.g., a transgenic mouse) capable of producing such single-domain antibodies or VHH sequences; or synthetic or semi-synthetic amino acid sequences or single-domain antibodies; including, but not limited to, partially humanized single-domain antibodies or VHH sequences, fully humanized single-domain antibodies or VHH sequences, camelized heavy chain variable (VH) domain sequences, as well as single-domain antibodies obtained by any suitable techniques, such as those described herein.

A humanized single-domain antibody, may consists of four framework regions (FR1, FR2, FR3, FR4) and three complementarity determining regions (CDR1, CDR2, CDR3), in which CDR1, CDR2, and CDR3 are as described herein and in which said humanized single-domain antibody comprises at least one humanizing substitution, and in particular at least one humanizing substitution in at least one of its framework sequences.

A single-domain antibody may comprise CDR sequences having at least 70% identity, at least 80% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity with the CDR sequences of at least one of the amino acid sequences of SEQ ID NOS: 1 to 15 (see Table 2). This degree of identity can for example be determined by determining the degree of amino acid identity (in a manner described herein) between said single-domain antibody and one or more of the sequences of SEQ ID NOS: 1 to 15 (Table 2), in which the amino acid residues that form the framework regions are disregarded. Such single-domain antibodies can be as further described herein.

A single-domain antibody may comprise an amino acid sequence that is selected from the group consisting of SEQ ID NOS: 1 to 15 (see Table 2), or selected from the group consisting of an amino acid sequence having at least 70%, at least 80%, at least 90%, at least 95%, at least 96%, at least 97%, at least 98%, or at least 99% identity with at least one of the amino acid sequences of SEQ ID NOS: 1 to 15 (see Table 2).

Humanized and/or sequences may comprise optimized variants of the single-domain antibodies of SEQ ID NOS: 1 to 15 (see Table 2), that comprise at least one humanizing and/or sequence-optimizing substitution compared to the corresponding native VHH sequence, and in particular at least one humanizing and/or sequence-optimizing substitution in at least one of its framework sequences.

The single-domain antibody or fragment thereof may comprise a VHH domain encoded by a nucleotide sequence of any one of SEQ ID NOS: 97-111 (see Table 3).

The single-domain antibody or fragment thereof comprises one, two, or all three CDRS of the VHH domain encoded by a nucleotide sequence of any one of SEQ ID NOS: 97-111 (see Table 3) and/or one, two, three, or all four framework regions (FR) of the VHH domain encoded by a nucleotide sequence of any one of SEQ ID NOS: 97-111 (see Table 3).

### Polypeptides

The polypeptides described herein may comprise or consist essentially of at least one single-domain antibody described herein. Examples of polypeptides described herein are given in SEQ ID NOS: 112 to 117 (see Table 4) and SEQ ID NOS: 118 to 123 (see Table 5).

It will be clear to the skilled person that the single-domain antibodies that are described herein as “preferred” (or “more preferred”, “even more preferred”, etc.) are also preferred (or more preferred, or even more preferred, etc.) for use in the polypeptides described herein. Thus, polypeptides that comprise or consist essentially of one or more “preferred” single-domain antibodies described herein will generally be preferred, and polypeptides that comprise or consist essentially of one or more “more preferred” single-domain antibodies described herein will generally be more preferred, etc.

Generally, proteins or polypeptides that comprise or consist essentially of only one single-domain antibody (e.g., only one single-domain antibody described herein) will be referred to herein as “monovalent” proteins or polypeptides or as “monovalent constructs”. Proteins and polypeptides that comprise or consist essentially of two or more single-domain antibodies (e.g., at least two single-domain antibodies described herein or at least one single-domain antibodies described herein and at least one other single-domain antibody) will be referred to herein as “multivalent” proteins or polypeptides or as “multivalent constructs”, and these may provide certain advantages compared to the corresponding monovalent single-domain antibodies described herein. Some non-limiting examples of such multivalent constructs are described herein.

A polypeptide described herein may comprise or consist essentially of at least two single-domain antibodies described herein, such as two or three single-domain antibodies described herein. As further described herein, such multivalent constructs can provide certain advantages compared to a protein or polypeptide comprising or essentially consisting of only one single-domain antibodies described herein, for example, improved avidity for SARS-CoV-2.

A polypeptide described herein may comprise or consist essentially of at least one single-domain antibody described herein and at least one other binding unit (e.g., directed against another epitope, antigen, target, protein or polypeptide), which is preferably also a single-domain antibody. Such proteins or polypeptides are also referred to herein as “multispecific” proteins or polypeptides or as “multispecific constructs”, and these may provide certain advantages compared to the corresponding monovalent single-domain antibodies described herein.

A polypeptide described herein may comprise or consist essentially of at least one single-domain antibody described herein, optionally one or more further single-domain antibodies, and at least one other amino acid sequence (e.g., a protein or polypeptide) that confers at least one desired property to the single-domain antibody described herein and/or to the resulting fusion protein. Again, such fusion proteins may provide certain advantages compared to the corresponding monovalent single-domain antibody described herein. Some non-limiting examples of such amino acid sequences and of such fusion constructs are described herein.

It is also possible to combine two or more of the above aspects, for example, to provide a trivalent bispecific construct comprising two single-domain antibodies described herein and one other single-domain antibody, and optionally one or more other amino acid sequences. Further non-limiting examples of such constructs, as well as some constructs that are preferred within the context described herein, are described herein.

In the above constructs, the one or more single-domain antibodies and/or other amino acid sequences may be directly linked to each other and/or linked to each other via one or more linker sequences. Some suitable, but non-limiting, examples of such linkers are described herein.

A single-domain antibody described herein or a compound, construct or polypeptide may comprise at least one single-domain antibody described herein may have an increased half-life compared to the corresponding amino acid sequence described herein.

Single-domain antibody sequences or polypeptides described herein may be chemically modified to increase the half-life thereof (for example, by means of pegylation); amino acid sequences described herein that comprise at least one additional binding site for binding to a serum protein (e.g., serum albumin); or polypeptides described herein that comprise at least one single-domain antibody described herein that is linked to at least one moiety (and in particular at least one amino acid sequence) that increases the half-life of the single-domain antibody described herein. Examples of polypeptides described herein that comprise such half-life extending moieties or amino acid sequences for example include, without limitation, polypeptides in which the one or more single-domain antibodies described herein are linked to one or more serum proteins or fragments thereof (e.g., serum albumin or suitable fragments thereof) or to one or more binding units that can bind to serum proteins (for example, single-domain antibodies that can bind to serum proteins such as serum albumin, serum immunoglobulins such as IgG, or transferrin); polypeptides in which a single-domain antibody described herein is linked to an Fc portion (e.g., a human Fc) or a suitable part or fragment thereof; or polypeptides in which the one or more single-domain antibodies described herein are linked to one or more small proteins or peptides that can bind to serum proteins (e.g., the proteins and peptides described in WO 91/01743, WO 01/45746, WO 02/076489 and WO 2008/068280.

Again, single-domain antibodies, compounds, constructs or polypeptides may comprise one or more additional groups, residues, moieties or binding units, such as one or more further amino acid sequences and in particular one or more additional single-domain antibodies (e.g., not directed against SARS-CoV-2), so as to provide a tri- or multispecific single-domain antibody construct.

Generally, the single-domain antibody described herein (or compounds, constructs or polypeptides comprising the same) with increased half-life may have a half-life that is at least 1.5 times, preferably at least 2 times, such as at least 5 times, at least 10 times, or more than 20 times, greater than the half-life of the corresponding amino acid sequence described herein. For example, the single-domain antibodies, compounds, constructs or polypeptides described herein with increased half-life may have a half-life that is increased by more than 1 hour, preferably more than 2 hours, more than 6 hours, such as more than 12 hours, or more than 24, 48 or 72 hours, compared to the corresponding amino acid sequence described herein.

Single-domain antibodies, compounds, constructs or polypeptides described herein may exhibit a serum half-life in humans of at least about 12 hours, at least 24 hours, at least 48 hours, at least 72 hours or more. For example, compounds or polypeptides described herein may have a half-life of at least 5 days, such as about 5 to 10 days, preferably at least 9 days, such as about 9 to 14 days, or at least about 10 days, such as about 10 to 15 days, or at least about 11 days, such as about 11 to 16 days), or at least about 12 days, such as about 12 to 18 days or more, or more than 14 days, such as about 14 to 19 days.

Polypeptides comprising one or more single-domain antibodies described herein may bind to SARS-CoV-2:
with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter;
   and/or
with a kₒₙ rate of between 10² M⁻¹ s⁻¹ to about 10⁷ M⁻¹s⁻¹, preferably between 10³ M⁻¹ s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹ s⁻¹;
   and/or
with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

A polypeptide may comprise only one amino acid sequence described herein is preferably such that it will bind to SARS-CoV-2 with an affinity less than 500 nM, preferably less than 200 nM, more preferably less than 10 nM, such as less than 500 pM. A polypeptide that contains two or more amino acid sequences described herein may bind to SARS-CoV-2 with an increased avidity compared to a polypeptide that contains only one amino acid sequence described herein.

Some preferred IC₅₀ values for binding of the amino acid sequences or polypeptides described herein to SARS-CoV-2 are described herein.

Polypeptides according to this aspect may, for example, be selected from the group consisting of amino acid sequences that are at least 70%, at least 80%, at least 90%, at least 95%, at least 96%, at least 97%, at least 98%, or at least 99% or more identical to one or more of the amino acid sequences of SEQ ID NOS: 112 to 117 (see Table 4) and SEQ ID NOS: 118 to 123 (Table 5), in which the single-domain antibodies comprised within said amino acid sequences are preferably as further defined herein.

A nucleic acid encoding an amino acid sequence described herein (e.g., a single-domain antibody described herein) or a polypeptide comprising the same is described herein. In an embodiment, the nucleic acid encoding an amino acid sequence described herein may comprise one or more of the nucleotide sequences of SEQ ID NOS: 97 to 111 (see Table 3). Again, as generally described herein for the nucleic acids of the present disclosure, such a nucleic acid may be in the form of a genetic construct, as described herein.

A host or host cell that expresses or that is capable of expressing an amino acid sequence (e.g., a single-domain antibody) described herein and/or a polypeptide comprising the same; and/or that contains a nucleic acid of the present disclosure is described herein.

A product or composition may comprise at least one amino acid sequence described herein, at least one polypeptide described herein and/or at least one nucleic acid described herein, and optionally one or more further components of such compositions known per se, e.g., depending on the intended use of the composition. Such a product or composition may, for example, be a pharmaceutical composition, a veterinary composition, or a product or composition for diagnostic use.

Methods for preparing or generating the amino acid sequences, compounds, constructs, polypeptides, nucleic acids, host cells, products and compositions are described herein.

Applications and uses of the amino acid sequences, compounds, constructs, polypeptides, nucleic acids, host cells, products and compositions are described herein, including methods for the prevention and/or treatment for diseases and disorders associated with SARS-CoV-2.

Generally, it should be noted that the term single-domain antibody, as used herein, in its broadest sense is not limited to a specific biological source or to a specific method of preparation. For example, as discussed in more detail below, the single-domain antibodies described herein can generally be obtained by any suitable technique. One preferred class of single-domain antibodies corresponds to VHH domains of naturally-occurring heavy chain antibodies directed against SARS-CoV-2. As further described herein, such VHH sequences can generally be generated or obtained by immunizing a species of Camelid with SARS-CoV-2 polypeptide(s) (e.g., to raise an immune response and/or heavy chain antibodies directed against SARS-CoV-2), by obtaining a suitable biological sample from said Camelid (e.g., a blood sample, serum sample, or sample of B cells), and by generating VHH sequences directed against SARS-CoV-2, starting from said sample, using any suitable technique.

Alternatively, such naturally-occurring VHH domains against SARS-CoV-2, can be obtained from naive libraries of Camelid VHH sequences, for example, by screening such a library using SARS-CoV-2, or at least one part, fragment, antigenic determinant or epitope thereof, using one or more known screening techniques. Such libraries and techniques are, for example, described in WO 99/37681, WO 01/90190, WO 03/025020, and WO 03/035694. Alternatively, improved synthetic or semi-synthetic libraries derived from naive VHH libraries may be used, such as VHH libraries obtained from naive VHH libraries by techniques such as random mutagenesis and/or CDR shuffling, as, for example, described in WO 00/43507.

A class of single-domain antibodies described herein may comprise single-domain antibodies with an amino acid sequence that corresponds to the amino acid sequence of a naturally-occurring VHH domain, but that has been “humanized”, e.g., by replacing one or more amino acid residues in the amino acid sequence of said naturally-occurring VHH sequence (and in particular in the framework sequences) by one or more of the amino acid residues that occur at the corresponding position(s) in a VH domain from a conventional four-chain antibody from a human. Another class of single-domain antibodies described herein may comprise single-domain antibodies with an amino acid sequence that corresponds to the amino acid sequence of a naturally-occurring VH domain, but that has been “camelized”, e.g., by replacing one or more amino acid residues in the amino acid sequence of a naturally-occurring VH domain from a conventional four-chain antibody by one or more of the amino acid residues that occur at the corresponding position(s) in a VHH domain of a heavy chain antibody.

In addition to humanizing or camelizing substitutions as described herein, the amino acid sequences described herein may comprise one or more other/further substitutions. In an embodiment, such other/further substitutions may include or consist essentially of one or more of the following substitutions:
a) one or more conservative amino acid substitutions; and/or
b) one or more substitutions in which a “camelid” amino acid residue at a certain position is replaced by a different “camelid” amino acid residue that occurs at said position; and/or
c) one or more substitutions that improve the properties of the protein, such as substitutions that improve the long-term stability and/or properties under storage of the protein.
   These may include, but are not limited to, substitutions that prevent or reduce oxidation events (e.g., of methionine residues); that prevent or reduce pyroglutamate formation; and/or that prevent or reduce isomerisation or deamidation of aspartic acids or asparagines (e.g., of DG, DS, NG or NS motifs). Such substitutions are generally known by those of ordinary skill in the art.

Although the use of the single-domain antibodies described herein and of the polypeptides of the disclosure is preferred, it will be clear that on the basis of the description herein, the skilled person will also be able to design and/or generate, in an analogous manner, other amino acid sequences and in particular single-domain antibodies against SARS-CoV-2, as well as polypeptides comprising such single-domain antibodies.

For example, it will also be clear to the skilled person that it may be possible to “graft” one or more of the CDRs mentioned above for the single-domain antibodies described herein onto other single-domain antibodies or other protein scaffolds, including, but not limited to, human scaffolds or non-immunoglobulin scaffolds. Suitable scaffolds and techniques for such CDR grafting will be clear to the skilled person and are well known in the art. For example, techniques known for grafting mouse or rat CDRs onto human frameworks and scaffolds can be used in an analogous manner to provide chimeric proteins comprising one or more of the CDRs of the single-domain antibodies described herein and one or more human framework regions or sequences.

It should also be noted that, when the single-domain antibodies described herein comprise one or more other CDR sequences than the preferred CDR sequences mentioned above, these CDR sequences can be obtained in any known manner.

Other suitable methods and techniques for obtaining the single-domain antibodies described herein and/or nucleic acids encoding the same, starting from naturally-occurring VH sequences or preferably VHH sequences, will be clear to the skilled person.

Anti-SARS-CoV-2 single-domain antibodies may be used in the treatment of diseases or disorders in which a partial or total blockade and/or neutralization of SARS-CoV-2 activity is desired. In an embodiment, the anti-SARS-CoV-2 single-domain antibodies described herein are used to treat COVID-19 and/or diseases and disorders associated with, or resulting from SARS-CoV-2 infection. In an embodiment, the anti-SARS-CoV-2 single-domain antibodies described herein are used to treat acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

In another aspect, the anti-SARS-CoV-2 single-domain antibodies described herein find utility as reagents for detection and isolation of SARS-CoV-2, such as detection and/or quantification of SARS-CoV-2 expression in various cells and/or tissues. The anti-SARS-CoV-2 single-domain antibodies described herein can be used in SARS-CoV-2 receptor binding domain (RBD) binding assays to screen for antagonists of SARS-CoV-2 which will exhibit similar pharmacological effects.

The single-domain antibodies described herein may immunospecifically bind a polypeptide comprising the amino acid sequence of SEQ ID NO: 124, 125, 126, 127, 128, or a combination thereof, or a polypeptide comprising a portion (e.g., a fragment) of the amino acid sequence of SEQ ID NO: 124, 125, 126, 127, 128, or a combination thereof. The single-domain antibodies described herein include molecules comprising, or alternatively consisting of, antibody fragments or variants thereof that immunospecifically bind to a receptor binding domain (RBD) of a coronavirus amino acid sequence (e.g., a polypeptide comprising, or alternatively consisting of, amino acids 331-524 of the Spike protein of SARS-CoV-2 (SEQ ID NO: 124), amino acid residues 318-510 of the Spike protein of SARS-CoV (SEQ ID NO: 125), amino acid residues 377-588 of the MERS-CoV spike protein (SEQ ID NO: 126), and/or amino acids 319-541 of the SARS CoV-2 spike receptor-binding domain (SEQ ID NO: 127)).

Moreover, polypeptide fragments that may be bound by single-domain antibodies described herein can be at least 10, 20, 30, 40, 50, 60, 70, 80, 90, 100, 110, 120, 130, 140, 150, 175 or 200 amino acids in length. In this context, “about” means the particularly recited ranges and ranges larger or smaller by several, a few, 5, 4, 3, 2 or 1 amino acid residues at either or both the amino- and carboxy-termini.

Single-domain antibodies that bind SARS-CoV-2 polypeptide fragments may comprise or consist of functional regions of polypeptides described herein, such as the Gamier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index. In a preferred embodiment, the polypeptide fragments bound by the single-domain antibodies described herein are antigenic (e.g., comprising four or more contiguous amino acids having an antigenic index of greater than or equal to 1.5, as identified using the default parameters of the Jameson-Wolf program) of a coronavirus receptor binding domain polypeptide (e.g., SEQ ID NO: 124, 125, 126, 127).

### Antibody Epitopes

The single-domain antibodies that bind a polypeptide may comprise, or alternatively consist of, an epitope-bearing portion of a polypeptide described herein. The epitope of this polypeptide portion may be an immunogenic or antigenic epitope of a polypeptide described herein. An “immunogenic epitope” is defined as a part of a protein that elicits an antibody response when the whole protein is the immunogen. The region of a protein molecule to which an antibody can bind may also be defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of polypeptides bearing an antigenic epitope (e.g., that comprise a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Leamer, R. A. (1983) “Antibodies that react with predetermined sites on proteins”, Science, 219:660-666. Peptides capable of eliciting protein-reactive sera are frequently represented in the primary sequence of a protein, can be characterized by a set of simple chemical rules, and are confined neither to immunodominant regions of intact proteins (e.g., immunogenic epitopes) nor to the amino or carboxyl terminals. Antigenic epitope-bearing peptides and polypeptides described herein are therefore useful to raise antibodies, including single-domain antibodies, that bind specifically to a polypeptide described herein. See, for instance, Wilson et al., Cell 37:767-778 (1984) at 777.

Single-domain antibodies described herein bind antigenic epitope-bearing peptides and polypeptides of SARS-CoV-2 (e.g., the SARS-CoV-2 RBD) and preferably comprise a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids contained within the amino acid sequence of a SARS-CoV-2 polypeptide (e.g., a SARS-CoV-2 RBD polypeptide). Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof.

Single-domain antibodies that bind polypeptides may comprise, or alternatively consist of, an epitope of a coronavirus amino acid sequence. Preferably, the epitope is within the receptor binding domain of the coronavirus amino acid sequence. For example, single-domain antibodies described herein may bind polypeptides comprising, or alternatively consisting of, an epitope contained within the polypeptide having an amino acid sequence of SEQ ID NO: 124, 125, 126, 127, 128, or a combination thereof.

The single-domain antibodies and antigen binding fragments described herein preferably bind to an epitope consisting of 6 to 12 residues within the receptor binding domain of amino acids 331-524 of the Spike protein of SARS-CoV-2 (SEQ ID NO: 124), amino acid residues 318-510 of the Spike protein of SARS-CoV (SEQ ID NO: 125), amino acid residues 377-588 of the MERS-CoV spike protein (SEQ ID NO: 126), the receptor binding domain of amino acids 319-541 of the Spike protein of SARS-CoV-2 (SEQ ID NO: 127), and/or the Spike protein of SARS-CoV-2 (SEQ ID NO 128).

The single-domain antibodies and antigen binding fragments described herein preferably bind to an epitope consisting of 6 to 12 residues within the receptor binding domain of amino acids 319-541 of the SARS CoV-2 spike receptor-binding domain (SEQ ID NO: 127):

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNF

The term “epitopes,” as used herein, refers broadly to portions of a polypeptide having antigenic or immunogenic activity in an animal, preferably a mammal, and most preferably in a human. In a preferred embodiment, the present disclosure encompasses single-domain antibodies that bind a polypeptide comprising an epitope. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Antigenic epitopes are useful, for example, to raise antibodies, including single-domain antibodies, that specifically bind the epitope. Preferred antigenic epitopes include, but are not limited to, the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes of SARS-CoV-2 may be presented for eliciting an antibody response together with a carrier protein, such as an albumin, to an animal system (e.g., rabbit, mouse, or camelid), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

SARS-CoV-2 polypeptide fragments that function as epitopes may be produced by any conventional means. (See, e.g., Houghten, Proc. Natl. Acad. Sci. USA 82:5131-5135 (1985) and U.S. Pat. No. 4,631,211).

Epitope-bearing SARS-CoV-2 polypeptides may be used to induce single-domain antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, antipeptide antibody titer may be boosted by coupling the peptide to a macromolecular carrier, such as keyhole limpet hemocyanin (KLH) or tetanus toxoid. For instance, peptides comprising cysteine residues may be coupled to a carrier using a linker such as maleimidobenzoyl-N-hydroxysuccinimide ester (MBS), while other peptides may be coupled to carriers using a more general linking agent such as glutaraldehyde. Animals (e.g., llamas, camels, and alpacas) are immunized with either free or carrier-coupled peptides, for instance, by intraperitoneal and/or subcutaneous injection of emulsions comprising about 0.5 to 100 milligrams of peptide or carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

### Anti-SARS-CoV-2 Single-Domain Antibody Fusion Proteins

Derivatives of the single-domain antibodies are also described herein. Such derivatives can generally be obtained by modification, and in particular by chemical and/or biological (e.g., enzymatic) modification, of the single-domain antibodies described herein and/or of one or more of the amino acid residues that form the single-domain antibodies described herein.

Examples of such modifications, as well as examples of amino acid residues within the single-domain antibody sequence that can be modified in such a manner (e.g., either on the protein backbone but preferably on a side chain), methods and techniques that can be used to introduce such modifications and the potential uses and advantages of such modifications will be clear to the skilled person.

For example, such a modification may involve the introduction (e.g., by covalent linking or in any other suitable manner) of one or more functional groups, residues or moieties into or onto the single-domain antibodies described herein, and in particular of one or more functional groups, residues or moieties that confer one or more desired properties or functionalities to the single-domain antibodies described herein. Examples of such functional groups will be clear to the skilled person.

For example, such modification may comprise the introduction (e.g., by covalent binding or in any other suitable manner) of one or more functional groups that increase the half-life, the solubility and/or the absorption of the single-domain antibodies described herein, that reduce the immunogenicity and/or the toxicity of the single-domain antibodies described herein, that eliminate or attenuate any undesirable side effects of the single-domain antibodies described herein, and/or that confer other advantageous properties to and/or reduce the undesired properties of the single-domain antibodies and/or polypeptides described herein; or any combination of two or more of the foregoing. Examples of such functional groups and of techniques for introducing them will be clear to the skilled person, and include, but are not limited to, known functional groups and techniques for the modification of pharmaceutical proteins, and in particular for the modification of antibodies or antibody fragments (including scFvs and single-domain antibodies), for which reference is for example made to Remington's Pharmaceutical Sciences, 16th ed., Mack Publishing Co., Easton, PA (1980). Such functional groups may, for example, be linked directly (e.g., covalently) to a single-domain antibody described herein, or optionally via a suitable linker or spacer, as will again be clear to the skilled person.

One of the most widely used techniques for increasing the half-life and/or reducing the immunogenicity of pharmaceutical proteins comprises attachment of a suitable pharmacologically acceptable polymer, such as poly(ethyleneglycol) (PEG) or derivatives thereof (e.g., methoxypoly(ethyleneglycol) or mPEG). Generally, any suitable form of pegylation can be used, such as the pegylation used in the art for antibodies and antibody fragments (including but not limited to (single) domain antibodies and scFvs); reference is made to for example Chapman, Nat. Biotechnol., 54, 531-545 (2002); Veronese and Harris, Adv. Drug Deliv. Rev. 54, 453-456 (2003), Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see, e.g., Yang et al., Protein Engineering, 16, 10, 761-770 (2003)). For example, for this purpose, PEG may be attached to a cysteine residue that naturally occurs in a single-domain antibody described herein, a single-domain antibody described herein may be modified so as to introduce one or more cysteine residues for attachment of PEG, or an amino acid sequence comprising one or more cysteine residues for attachment of PEG may be fused to the N- and/or C-terminus of a single-domain antibody described herein, all using known techniques of protein engineering.

Preferably, for the single-domain antibodies and proteins described herein, a PEG is used with a molecular weight of more than 5000, such as more than 10,000 and less than 200,000, such as less than 100,000; for example, in the range of 20,000-80,000.

Another modification comprises N-linked or O-linked glycosylation, usually as part of co-translational and/or post-translational modification, depending on the host cell used for expressing the single-domain antibody or polypeptide described herein.

Yet another modification may comprise the introduction of one or more detectable labels or other signal-generating groups or moieties, depending on the intended use of the labelled single-domain antibody. Suitable labels and techniques for attaching, using and detecting them will be clear to the skilled person, and for example include, but are not limited to, fluorescent labels, phosphorescent labels, chemiluminescent labels, bioluminescent labels, radioisotopes, metals, metal chelates, metallic cations, chromophores, and enzymes. Other suitable labels will be clear to the skilled person, and for example include moieties that can be detected using NMR or ESR spectroscopy.

Such labelled single-domain antibodies and polypeptides described herein may, for example, be used for in vitro, in vivo, or in situ assays (including immunoassays such as ELISA, RIA, EIA and other “sandwich assays”) as well as in vivo diagnostic and imaging purposes, depending on the choice of the specific label.

A modification may involve the introduction of a chelating group, for example to chelate one of the metals or metallic cations referred to above. Suitable chelating groups include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the single-domain antibody of the present invention to another protein, polypeptide or chemical compound that is bound to the other half of the binding pair, e.g., through formation of the binding pair. For example, a single-domain antibody described herein may be conjugated to biotin, and linked to another protein, polypeptide, compound or carrier conjugated to avidin or streptavidin. For example, such a conjugated single-domain antibody may be used as a reporter, for example in a diagnostic system where a detectable signal-producing agent is conjugated to avidin or streptavidin. Such binding pairs may, for example, also be used to bind the single-domain antibody of the present invention to a carrier, including carriers suitable for pharmaceutical purposes. One non-limiting example is the liposomal formulations described by Cao and Suresh, Journal of Drug Targeting, 8, 4, 257 (2000). Such binding pairs may also be used to link a therapeutically active agent to the single-domain antibodies described herein.

Other potential chemical and enzymatic modifications will be clear to the skilled person. Such modifications may also be introduced for research purposes (e.g., to study function-activity relationships).

Preferably, the derivatives are such that they bind to SARS-CoV-2 with an affinity (measured and/or expressed as a K_{D} value (actual or apparent), a K_{A} value (actual or apparent), a kₒₙ rate and/or a k_{off} rate, or alternatively as an IC50 value, as further described herein) that is as described herein for the single-domain antibodies described herein.

Proteins or polypeptides may consist essentially of or comprise at least one single-domain antibody described herein.

Said amino acid residues may or may not change, alter, or otherwise influence the biological properties of the single-domain antibody and may or may not add further functionality to the single-domain antibody. For example, such amino acid residues can:
comprise an N-terminal Met residue, for example as result of expression in a heterologous host cell or host organism;
may form a signal sequence or leader sequence that directs secretion of the single-domain antibody from a host cell upon synthesis. Suitable secretory leader peptides will be clear to the skilled person, and may be as further described herein. Usually, such a leader sequence will be linked to the N-terminus of the single-domain antibody, although the present disclosure in its broadest sense is not limited thereto;
may form a sequence or signal that allows the single-domain antibody to be directed towards and/or to penetrate or enter into specific organs, tissues, cells, or parts or compartments of cells, and/or that allows the single-domain antibody to penetrate or cross a biological barrier such as a cell membrane, a cell layer such as a layer of epithelial cells, a tumor including solid tumors, or the blood-brain-barrier;
may form a “tag”, for example, an amino acid sequence or residue that allows or facilitates the purification of the single-domain antibody, for example using affinity techniques directed against said sequence or residue. Thereafter, said sequence or residue may be removed (e.g., by chemical or enzymatic cleavage) to provide the single-domain antibody sequence (for this purpose, the tag may optionally be linked to the single-domain antibody sequence via a cleavable linker sequence or comprise a cleavable motif). Some preferred, but non-limiting, examples of such residues are multiple histidine residues, glutathione residues and a myc-tag;
may be one or more amino acid residues that have been functionalized and/or that can serve as a site for attachment of functional groups. Suitable amino acid residues and functional groups will be clear to the skilled person and include, but are not limited to, the amino acid residues and functional groups described herein for the derivatives of the single-domain antibodies described herein.

According to another aspect, a polypeptide described herein comprises a single-domain antibody described herein, which is fused at its amino terminal end, at its carboxy terminal end, or both at its amino terminal end and at its carboxy terminal end to at least one further amino acid sequence, e.g., so as to provide a fusion protein comprising said single-domain antibody described herein and the one or more further amino acid sequences. Such a fusion will also be referred to herein as a “single-domain antibody fusion”.

The one or more further amino acid sequence may be any suitable and/or desired amino acid sequences. The further amino acid sequences may or may not change, alter or otherwise influence the properties of the single-domain antibody, and may or may not add further functionality to the single-domain antibody or the polypeptide described herein. Preferably, the further amino acid sequence is such that it confers one or more desired properties or functionalities to the single-domain antibody or the polypeptide described herein.

For example, the further amino acid sequence may also provide a second binding site, which may be directed against any desired protein, polypeptide, antigen, antigenic determinant or epitope (including, but not limited to, the same protein, polypeptide, antigen, antigenic determinant or epitope against which the single-domain antibody described herein is directed, or a different protein, polypeptide, antigen, antigenic determinant or epitope).

Examples of such amino acid sequences will be clear to the skilled person, and may generally comprise all amino acid sequences that are used in peptide fusions based on conventional antibodies and fragments thereof (including, but not limited to, scFvs and single-domain antibodies). Reference is, for example, made to the review by Holliger and Hudson, Nature Biotechnology, 23, 9, 1126-1136 (2005).

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides described herein compared to the single-domain antibodies described herein. Some non-limiting examples of such amino acid sequences are serum proteins, such as human serum albumin (see, e.g., WO 00/27435) or haptenic molecules (e.g., haptens that are recognized by circulating antibodies, see e.g., WO 98/22141).

In particular, it has been described that linking fragments of immunoglobulins (e.g., VH domains) to serum albumin or to fragments thereof can be used to increase the half-life (see, e.g., WO 00/27435 and WO 01/077137). The single-domain antibody described herein is preferably either directly linked to serum albumin (or to a suitable fragment thereof) or via a suitable linker, and in particular via a suitable peptide linked so that the polypeptide described herein can be expressed as a genetic fusion (protein). According to one aspect, the single-domain antibody described herein may be linked to a fragment of serum albumin that at least comprises the domain Ill of serum albumin or part thereof.

The further amino acid sequence may provide a second binding site or binding unit that is directed against a serum protein (e.g., human serum albumin or another serum protein such as IgG), so as to provide increased half-life in serum. Such amino acid sequences, for example, include the small peptides and binding proteins described in WO 91/01743, WO 01/45746 and WO 02/076489 and the dAbs described in WO 03/002609 and WO 04/003019.

The one or more further amino acid sequences may comprise one or more parts, fragments or domains of conventional four-chain antibodies (and in particular human antibodies) and/or of heavy chain antibodies. For example, a single-domain antibody described herein may be linked to a conventional (preferably human) VH or VL domain or to a natural or synthetic analog of a VH or VL domain, optionally via a linker sequence (including, but not limited to, other (single) domain antibodies, such as the dAbs described by Ward et al.).

The at least one single-domain antibody may also be linked to one or more (preferably human) constant heavy 1 (CH1), constant heavy 2 (CH2) and/or constant heavy 3 (CH3) domains, optionally via a linker sequence. For instance, a single-domain antibody linked to a suitable CH1 domain could for example be used—together with suitable light chains—to generate antibody fragments/structures analogous to conventional Fab fragments or F(ab′)₂ fragments, but in which one or (in case of an F(ab′)₂ fragment) one or both of the conventional VH domains have been replaced by a single-domain antibody described herein. Also, two single-chain antibodies could be linked to a CH3 domain (optionally via a linker) to provide a construct with increased half-life in vivo.

One or more single-domain antibodies described herein may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide described herein and/or may confer the ability to bind to one or more Fc receptors. For example, for this purpose, and without being limited thereto, the one or more further amino acid sequences may comprise one or more CH2 and/or CH3 domains of an antibody, such as from a heavy chain antibody (as described herein) and more preferably from a conventional human four-chain antibody; and/or may form (part of) an Fc region, for example from IgG (e.g., from IgG1, IgG2, IgG3 or IgG4), from IgE or from another human Ig such as IgA, IgD or IgM. For example, WO 94/04678 describes heavy chain antibodies comprising a Camelid VHH domain or a humanized derivative thereof, in which the Camelidae CH2 and/or CH3 domain have been replaced by human CH2 and CH3 domains, so as to provide an immunoglobulin that consists of 2 heavy chains each comprising a VHH domain (e.g., single-domain antibody) and human CH2 and CH3 domains (but no CH1 domain), which immunoglobulin has the effector function provided by the CH2 and CH3 domains and which immunoglobulin can function without the presence of any light chains. Other amino acid sequences that can be linked to the single-domain antibodies described herein to provide an effector function will be clear to the skilled person, and may be chosen on the basis of the desired effector function(s). Reference is, for example, made to WO 04/058820, WO 99/42077, WO 02/056910 and WO 05/017148, as well as the review by Holliger and Hudson, supra). Coupling of a single-domain antibody described herein to an Fc portion may lead to an increased half-life, compared to the corresponding single-domain antibody described herein. For some applications, the use of an Fc portion and/or of constant domains (e.g., CH2 and/or CH3 domains) that confer increased half-life without any biologically significant effector function may also be suitable or even preferred. Other suitable constructs comprising one or more single-domain antibodies and one or more constant domains with increased half-life in vivo will be clear to the skilled person, and may, for example, comprise two single-domain antibodies linked to a CH3 domain, optionally via a linker sequence. Generally, any fusion protein or derivatives with increased half-life will preferably have a molecular weight of more than 50 kD, the cut-off value for renal absorption.

Exemplary constructs comprising a single-domain antibody described herein and an Fc region from an IgG are shown below in Table 4.

TABLE 4
Amino Acid Sequences of Exemplary Anti-SARS-COV-2 Monomeric Fc
Fusion Constructs.

| | SEQ | |
|---|---|---|
| | ID | |
| ID | NO | Amino Acid Sequence |
| Nb15- | 112 | QVQLQESGGGLVQAGGSLRVSCAASGLPFSDYLMGWFRQAPGKEREYVAA |
| IgG1 | | ISQNGGHTYADSVLGRFTISRDNAKNTVYLQMNMLTPGDTAVYSCAARRPG |
| | | GGRWDAAHDYNYWGQGTQVTVSSDKTHTCPPCPAPELLGGPSVFLFPPKP |
| | | KDTLMISRTPEVTCVWDVSHEDPEVKFNWYVDGVEVHNAKTKPREEQYNST |
| | | YRWSVLTVLHQDWLNGKEYKCKVSNKALPAPIEKTISKAKGQPREPQVYTLP |
| | | PSREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| Nb17- | 113 | QVQLQESGGGLVQTGGSLRLSCAASGRTFGIYRMGWFRQAPGKEREFVAAI |
| IgG1 | | TSSADTAQYRDSVKGRFAISRDNAKNTLYLQMNSLKPEDTAIYYCAARDPTTL |
| | | EYGNWGQGTQVTVSSDKTHTCPPCPAPELLGGPSVFLFPPKPKDTLMISRT |
| | | PEVTCVWDVSHEDPEVKFNWYVDGVEVHNAKTKPREEQYNSTYRWSVLTV |
| | | LHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| Nb19- | 114 | QVQLQESGGGLVQAGGSLRLSCAASGSGFSIHAMGWYRQAPGKQREFVAV |
| IgG1 | | VGHKTNYADSVKGRFTISRDVGKNTVELQMNSLKVEDTAVYYCYCNTIVTMT |
| | | GVPDAVWGQGTQVTVSSDKTHTCPPCPAPELLGGPSVFLFPPKPKDTLMIS |
| | | RTPEVTCVWDVSHEDPEVKFNWYVDGVEVHNAKTKPREEQYNSTYRWSVL |
| | | TVLHQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| Nb56- | 115 | QVQLQESGGGLVQAGDSLRLSCVASERTFRRYGMGWFRQAPGKEREFVAA |
| IgG1 | | VDRSHTKTGYADFVKGRFTISTNYENMVYLQMNSLKPEDTAVYYCAAPSYEK |
| | | GSDPTSWNTDRGYDYWGQGTQVTVSSDKTHTCPPCPAPELLGGPSVFLFP |
| | | PKPKDTLMISRTPEVTCVWDVSHEDPEVKFNWYVDGVEVHNAKTKPREEQY |
| | | NSTYRWSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| msNb12- | 116 | QVKLEESGGGSVQAGGSLRLICTAPGLTHNNCGLDWYRRAPGKEREFVSSI |
| IgG2a | | SADGTTSYADSVKGRFTISKDKVEDTVYLQMNSLKPEDTAIYSCKTAFPYFGN |
| | | SCVLDYWGQGTSVTVSSEPKIPQPQPKPQPQPQPQPKPQPKPEPECTCPK |
| | | CPAPELLGGPSVFLFPPKPKDTLMISRTPEVTCVWDVSHEDPEVKFNWYVD |
| | | GVEVHNAKTKPREEQYNSTYRWSVLTVLHQDWLNGKEYKCKVSNKALPAPI |
| | | EKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHN |
| | | HYTQKSLSLSPGK |
| mNb30- | 117 | QVQLVESGGGLVQAGGSLRLSCAASGLTFSKYAMGWFRQAPGKERKFVATI |
| IgG2a | | SWSGDSAFYADSVKGRFTISRDNARNTVYLQMNSLKPEDTAVYYCAADRGM |
| | | GYGDFMDYWGQGTSVTASSEPKIPQPQPKPQPQPQPQPKPQPKPEPECTC |
| | | PKCPAPELLGGPSVFLFPPKPKDTLMISRTPEVTCVWDVSHEDPEVKFNWYV |
| | | DGVEVHNAKTKPREEQYNSTYRWSVLTVLHQDWLNGKEYKCKVSNKALPA |
| | | PIEKTISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEALH |
| | | NHYTQKSLSLSPGK |

Exemplary constructs comprising three single-domain antibodies described herein and an Fc region from an IgG are shown below in Table 5.

TABLE 5
Amino Acid Sequences of Exemplary Anti-SARS-COV-2 Trimeric Fc Fusion
Constructs.

| | SEQ | |
|---|---|---|
| | ID | |
| ID | NO | Amino Acid Sequence |
| Nb15- | 118 | QVQLQESGGGLVQAGGSLRVSCAASGLPFSDYLMGWFRQAPGKEREYVAA |
| tri- | | ISQNGGHTYADSVLGRFTISRDNAKNTVYLQMNMLTPGDTAVYSCAARRPG |
| IgG1 | | GGRWDAAHDYNYWGQGTQVTVSSGGGGSGGGGSGGGGSQVQLQESGG |
| | | GLVQAGGSLRVSCAASGLPFSDYLMGWFRQAPGKEREYVAAISQNGGHTY |
| | | ADSVLGRFTISRDNAKNTVYLQMNMLTPGDTAVYSCAARRPGGGRWDAAH |
| | | DYNYWGQGTQVTVSSGGGGSGGGGSGGGGSQVQLQESGGGLVQAGGSL |
| | | RVSCAASGLPFSDYLMGWFRQAPGKEREYVAAISQNGGHTYADSVLGRFTI |
| | | SRDNAKNTVYLQMNMLTPGDTAVYSCAARRPGGGRWDAAHDYNYWGQGT |
| | | QVTVSSDKTHTCPPCPAPELLGGPSVFLFPPKPKDTLMISRTPEVTCVWDVS |
| | | HEDPEVKFNWYVDGVEVHNAKTKPREEQYNSTYRWSVLTVLHQDWLNGKE |
| | | YKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNV |
| | | FSCSVMHEALHNHYTQKSLSLSPGK |
| Nb17- | 119 | QVQLQESGGGLVQTGGSLRLSCAASGRTFGIYRMGWFRQAPGKEREFVAAI |
| tri- | | TSSADTAQYRDSVKGRFAISRDNAKNTLYLQMNSLKPEDTAIYYCAARDPTTL |
| IgG1 | | EYGNWGQGTQVTVSSGGGGSGGGGSGGGGSQVQLQESGGGLVQTGGSL |
| | | RLSCAASGRTFGIYRMGWFRQAPGKEREFVAAITSSADTAQYRDSVKGRFAI |
| | | SRDNAKNTLYLQMNSLKPEDTAIYYCAARDPTTLEYGNWGQGTQVTVSSGG |
| | | GGSGGGGSGGGGSQVQLQESGGGLVQTGGSLRLSCAASGRTFGIYRMGW |
| | | FRQAPGKEREFVAAITSSADTAQYRDSVKGRFAISRDNAKNTLYLQMNSLKP |
| | | EDTAIYYCAARDPTTLEYGNWGQGTQVTVSSDKTHTCPPCPAPELLGGPSV |
| | | FLFPPKPKDTLMISRTPEVTCVWDVSHEDPEVKFNWYVDGVEVHNAKTKPR |
| | | EEQYNSTYRWSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Nb19- | 120 | QVQLQESGGGLVQAGGSLRLSCAASGSGFSIHAMGWYRQAPGKQREFVAV |
| tri- | | VGHKTNYADSVKGRFTISRDVGKNTVELQMNSLKVEDTAVYYCYCNTIVTMT |
| IgG1 | | GVPDAVWGQGTQVTVSSGGGGSGGGGSGGGGSQVQLQESGGGLVQAGG |
| | | SLRLSCAASGSGFSIHAMGWYRQAPGKQREFVAVVGHKTNYADSVKGRFTI |
| | | SRDVGKNTVELQMNSLKVEDTAVYYCYCNTIVTMTGVPDAVWGQGTQVTVS |
| | | SGGGGSGGGGSGGGGSQVQLQESGGGLVQAGGSLRLSCAASGSGFSIHA |
| | | MGWYRQAPGKQREFVAVVGHKTNYADSVKGRFTISRDVGKNTVELQMNSL |
| | | KVEDTAVYYCYCNTIVTMTGVPDAVWGQGTQVTVSSDKTHTCPPCPAPELL |
| | | GGPSVFLFPPKPKDTLMISRTPEVTCVWDVSHEDPEVKFNWYVDGVEVHNA |
| | | KTKPREEQYNSTYRWSVLTVLHQDWLNGKEYKCKVSNKALPAPIEKTISKAK |
| | | GQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLS |
| | | LSPGK |
| Nb56- | 121 | QVQLQESGGGLVQAGDSLRLSCVASERTFRRYGMGWFRQAPGKEREFVAA |
| tri- | | VDRSHTKTGYADFVKGRFTISTNYENMVYLQMNSLKPEDTAVYYCAAPSYEK |
| IgG1 | | GSDPTSWNTDRGYDYWGQGTQVTVSSGGGGGGGGSGGGGSQVQLQES |
| | | GGGLVQAGDSLRLSCVASERTFRRYGMGWFRQAPGKEREFVAAVDRSHTK |
| | | TGYADFVKGRFTISTNYENMVYLQMNSLKPEDTAVYYCAAPSYEKGSDPTS |
| | | WNTDRGYDYWGQGTQVTVSSGGGGSGGGGSGGGGSQVQLQESGGGLV |
| | | QAGDSLRLSCVASERTFRRYGMGWFRQAPGKEREFVAAVDRSHTKTGYAD |
| | | FVKGRFTISTNYENMVYLQMNSLKPEDTAVYYCAAPSYEKGSDPTSWNTDR |
| | | GYDYWGQGTQVTVSSDKTHTCPPCPAPELLGGPSVFLFPPKPKDTLMISRT |
| | | PEVTCVWDVSHEDPEVKFNWYVDGVEVHNAKTKPREEQYNSTYRWSVLTV |
| | | LHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| msNb12- | 122 | QVKLEESGGGSVQAGGSLRLICTAPGLTHNNCGLDWYRRAPGKEREFVSSI |
| tri- | | SADGTTSYADSVKGRFTISKDKVEDTVYLQMNSLKPEDTAIYSCKTAFPYFGN |
| IgG2a | | SCVLDYWGQGTSVTVSSGGGGSGGGGSGGGGSQVKLEESGGGSVQAGG |
| | | SLRLICTAPGLTHNNCGLDWYRRAPGKEREFVSSISADGTTSYADSVKGRFTI |
| | | SKDKVEDTVYLQMNSLKPEDTAIYSCKTAFPYFGNSCVLDYWGQGTSVTVS |
| | | SGGGGSGGGGSGGGGSQVKLEESGGGSVQAGGSLRLICTAPGLTHNNCGL |
| | | DWYRRAPGKEREFVSSISADGTTSYADSVKGRFTISKDKVEDTVYLQMNSLK |
| | | PEDTAIYSCKTAFPYFGNSCVLDYWGQGTSVTVSSEPKIPQPQPKPQPQPQ |
| | | PQPKPQPKPEPECTCPKCPAPELLGGPSVFLFPPKPKDTLMISRTPEVTCVW |
| | | DVSHEDPEVKFNWYVDGVEVHNAKTKPREEQYNSTYRWSVLTVLHQDWLN |
| | | GKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQ |
| | | GNVFSCSVMHEALHNHYTQKSLSLSPGK |
| mNb30- | 123 | QVQLVESGGGLVQAGGSLRLSCAASGLTFSKYAMGWFRQAPGKERKFVATI |
| tri- | | SWSGDSAFYADSVKGRFTISRDNARNTVYLQMNSLKPEDTAVYYCAADRGM |
| IgG2a | | GYGDFMDYWGQGTSVTASSGGGGSGGGGSGGGGSQVQLVESGGGLVQA |
| | | GGSLRLSCAASGLTFSKYAMGWFRQAPGKERKFVATISWSGDSAFYADSVK |
| | | GRFTISRDNARNTVYLQMNSLKPEDTAVYYCAADRGMGYGDFMDYWGQGT |
| | | SVTASSGGGGSGGGGSGGGGSQVQLVESGGGLVQAGGSLRLSCAASGLT |
| | | FSKYAMGWFRQAPGKERKFVATISWSGDSAFYADSVKGRFTISRDNARNTV |
| | | YLQMNSLKPEDTAVYYCAADRGMGYGDFMDYWGQGTSVTASSEPKIPQPQ |
| | | PKPQPQPQPQPKPQPKPEPECTCPKCPAPELLGGPSVFLFPPKPKDTLMISR |
| | | TPEVTCVWDVSHEDPEVKFNWYVDGVEVHNAKTKPREEQYNSTYRWSVLT |
| | | VLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLT |
| | | VDKSRWQQGNVFSCSVMHEALHNHYTQKSLSLSPGK |

In order to form a polypeptide described herein, one or more amino acid sequences described herein may be linked (optionally via a suitable linker or hinge region) to naturally occurring, synthetic or semisynthetic constant domains (or analogs, variants, mutants, parts or fragments thereof) that have a reduced (or essentially no) tendency to self-associate into dimers (e.g., compared to constant domains that naturally occur in conventional four-chain antibodies). Such monomeric (e.g., not self-associating) Fc chain variants, or fragments thereof, will be clear to the skilled person. For example, Helm et al., J Biol Chem 1996 271 7494, describe monomeric Fc chain variants that can be used in the polypeptide chains described herein.

Also, such monomeric Fc chain variants are preferably such that they are still capable of binding to the complement or the relevant Fc receptor(s) (depending on the Fc portion from which they are derived), and/or such that they still have some or all of the effector functions of the Fc portion from which they are derived (or at a reduced level still suitable for the intended use). Alternatively, in such a polypeptide chain described herein, the monomeric Fc chain may be used to confer increased half-life upon the polypeptide chain, in which case the monomeric Fc chain may also have no or essentially no effector functions.

Bivalent/multivalent, bispecific/multispecific or biparatopic/multiparatopic polypeptides described herein may also be linked to Fc portions.

The further amino acid sequences may also form a signal sequence or leader sequence that directs secretion of the single-domain antibody or the polypeptide described herein from a host cell upon synthesis (e.g., to provide a pre-, pro- or prepro-form of the polypeptide described herein, depending on the host cell used to express the polypeptide described herein).

The further amino acid sequence may also form a sequence or signal that allows the single-domain antibody or polypeptide described herein to be directed towards and/or to penetrate or enter into specific organs, tissues, cells, or parts or compartments of cells, and/or that allows the single-domain antibody or polypeptide described herein to penetrate or cross a biological barrier such as a cell membrane, a cell layer such as a layer of epithelial cells, a tumor including solid tumors, or the blood-brain barrier. Suitable examples of such amino acid sequences will be clear to the skilled person (see, e.g., WO 08/020079).

One or more further amino acid sequences comprise at least one further single-domain antibody, so as to provide a polypeptide described herein that comprises at least two, such as three, four, five or more single-domain antibodies, in which said single-domain antibodies may optionally be linked via one or more linker sequences. Polypeptides described herein that comprise two or more single-domain antibodies, of which at least one is a single-domain antibody described herein, are also referred to herein as “multivalent” polypeptides described herein, and the single-domain antibodies present in such polypeptides are also referred to herein as being in a “multivalent format”.

Polypeptides described herein may comprise at least two single-domain antibodies, in which at least one single-domain antibody is directed against a first antigen (e.g. against SARS-CoV-2) and at least one single-domain antibody is directed against a second antigen (e.g. different from SARS-CoV-2), are also referred to as “multispecific” polypeptides described herein, and the single-domain antibodies present in such polypeptides are also referred to herein as being in a “multispecific format”. Thus, for example, a “bispecific” polypeptide described herein is a polypeptide that comprises at least one single-domain antibody directed against a first antigen (e.g., SARS-CoV-2) and at least one further single-domain antibody directed against a second antigen (e.g., different from SARS-CoV-2), whereas a “trispecific” polypeptide described herein is a polypeptide that comprises at least one single-domain antibody directed against a first antigen (e.g., SARS-CoV-2), at least one further single-domain antibody directed against a second antigen (e.g., different from SARS-CoV-2), and at least one further single-domain antibody directed against a third antigen (e.g., different from both SARS-CoV-2 and the second antigen); etc.

Accordingly, one form of a bispecific polypeptide described herein is a bivalent polypeptide described herein comprising a first single-domain antibody directed against SARS-CoV-2 and a second single-domain antibody directed against a second antigen, in which said first and second single-domain antibodies may optionally be linked via a linker sequence; whereas a trispecific polypeptide described herein in its simplest form is a trivalent polypeptide described herein comprising a first single-domain antibody directed against SARS-CoV-2, a second single-domain antibody directed against a second antigen, and a third single-domain antibody directed against a third antigen, in which said first, second and third single-domain antibodies may optionally be linked via one or more, and in particular one and more, such as two, linker sequences.

A multispecific polypeptide described herein may comprise at least one single-domain antibody against SARS-CoV-2, and any number of single-domain antibody directed against one or more antigens different from SARS-CoV-2.

Furthermore, although the specific order or arrangement of the various single-domain antibodies in the polypeptides described herein may have some influence on the properties of the final polypeptide described herein (including, but not limited to, the affinity, specificity, or avidity for SARS-CoV-2, or against the one or more other antigens), said order or arrangement is usually not critical and may be chosen by the skilled person, optionally after some limited routine experiments based on the disclosure herein. Thus, when reference is made to a specific multivalent or multispecific polypeptide described herein, it should be noted that this encompasses any order or arrangements of the relevant single-domain antibodies, unless explicitly indicated otherwise.

The polypeptides described herein may comprise two or more single-domain antibodies and one or more further amino acid sequences (as described herein).

For multivalent and multispecific polypeptides comprising one or more VHH domains and their preparation, reference is also made to Conrath et al., J. Biol. Chem., Vol. 276, 10. 7346-7350, 2001; Muyldermans, Reviews in Molecular Biotechnology 74 (2001), 277-302; as well as to, for example, WO 96/34103 and WO 99/23221.

One non-limiting example of a multispecific polypeptide described herein comprises at least one single-domain antibody described herein and at least one single-domain antibody that provides for an increased half-life. Such single-domain antibodies may, for example, be single-domain antibodies that are directed against a serum protein, and in particular a human serum protein, such as human serum albumin, thyroxine-binding protein, (human) transferrin, fibrinogen, an immunoglobulin such as IgG, IgE or IgM, or against one of the serum proteins listed in WO 04/003019. Of these, single-domain antibodies that can bind to serum albumin (e.g., human serum albumin) or to IgG (e.g., human IgG) are preferred.

Generally, any polypeptides described herein with increased half-life that comprise one or more single-domain antibodies described herein, and any derivatives of single-domain antibodies described herein or of such polypeptides that have an increased half-life, preferably have a half-life that is at least 1.5 times, at least 2 times, at least 5 times, at least 10 times, or more than 20 times, greater than the half-life of the corresponding single-domain antibodies described herein. For example, such a derivative or polypeptides with increased half-life may have a half-life that is increased by more than 1 hour, more than 2 hours, more than 6 hours, more than 12 hours, or more than 24, 48 or 72 hours, compared to the corresponding single-domain antibodies described herein.

Derivatives or polypeptides may exhibit a serum half-life in human of at least about 12 hours, at least 24 hours, at least 48 hours, at least 72 hours or more. For example, such derivatives or polypeptides may have a half-life of at least 5 days (e.g., about 5 to 10 days), at least 9 days (e.g., about 9 to 14 days), at least about 10 days (e.g., about 10 to 15 days), or at least about 11 days (e.g., about 11 to 16 days), at least about 12 days (e.g., about 12 to 18 days or more), or more than 14 days (e.g., about 14 to 19 days).

In an embodiment, the polypeptides are capable of binding to one or more molecules which can increase the half-life of the polypeptide in vivo.

A multispecific polypeptide described herein may comprise at least one single-domain antibody described herein and at least one single-domain antibody that directs the polypeptide described herein towards, and/or that allows the polypeptide described herein to penetrate or to enter into specific organs, tissues, cells, or parts or compartments of cells, and/or that allows the single-domain antibody to penetrate or cross a biological barrier such as a cell membrane, a cell layer such as a layer of epithelial cells, a tumor including solid tumors, or the blood-brain-barrier. Examples of such single-domain antibodies include single-domain antibodies that are directed towards specific cell-surface proteins, markers or epitopes of the desired organ, tissue or cell, and single-domain brain-targeting antibody fragments.

In the polypeptides described herein, the one or more single-domain antibodies and the one or more polypeptides may be directly linked to each other and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in linking proteins or polypeptides that are intended for pharmaceutical use.

Some preferred spacers include the spacers and linkers that are used in the art to link antibody fragments or antibody domains. These include, for example, linkers that are used in the art to construct diabodies or scFv fragments. In this respect, however, it should be noted that, whereas in diabodies and in scFv fragments, the linker sequence used should have a length, a degree of flexibility and other properties that allow the pertinent VH and VL domains to come together to form the complete antigen-binding site, there is no particular limitation on the length or the flexibility of the linker used in the polypeptides described herein, since each single-domain antibody by itself forms a complete antigen-binding site.

For example, a linker may be a suitable amino acid sequence, and in particular amino acid sequences of between 1 and 50, preferably between 1 and 30, such as between 1 and 10 amino acid residues. Some preferred examples of such amino acid sequences include Gly-Ser linkers, for example of the type (GlyₓSer_{y})_{z} for example, (Gly₄Ser)₃ or (Gly₃Ser₂)₃, as well as hinge-like regions, such as the hinge regions of naturally occurring heavy chain antibodies or similar sequences (e.g., described in WO 94/04678). Other exemplary linkers are poly-alanine (e.g., AAA), as well as GS30 and GS9.

Other suitable linkers may comprise organic compounds or polymers, in particular those suitable for use in proteins for pharmaceutical use. For example, poly(ethyleneglycol) moieties have been used to link antibody domains.

The length and flexibility of the linkers and/or spacers can be chosen based on the polypeptides being linked and the desired properties thereof. For example, in a multispecific polypeptide described herein that comprises single-domain antibodies directed against two or more different antigenic determinants on the same antigen (e.g., against different epitopes of an antigen and/or against different subunits of a multimeric receptor, channel or protein), the length and flexibility of the linker are preferably such that it allows each single-domain antibody to bind to its intended antigenic determinant.

The linker(s) used confer one or more other favorable properties or functionality to the polypeptides described herein, and/or provide one or more sites for the formation of derivatives and/or for the attachment of functional groups (e.g., as described herein for the derivatives of the single-domain antibodies described herein). For example, linkers comprising one or more charged amino acid residues can provide improved hydrophilic properties, whereas linkers that form or comprise small epitopes or tags can be used for the purposes of detection, identification and/or purification.

When two or more linkers are used in the polypeptides described herein, these linkers may be the same or different.

For ease of expression and production, a polypeptide described herein may be a linear polypeptide. However, the present disclosure in its broadest sense is not limited thereto. For example, when a polypeptide described herein comprises three or more single-domain antibodies, it is possible to link them by use of a linker with three or more “arms”, which each “arm” being linked to a single-domain antibody, so as to provide a “star-shaped” construct. It is also possible, for example, to use circular constructs.

In an embodiment, the single-domain antibodies described herein may bind polypeptides comprising an immunogenic or antigenic epitope fused to other polypeptide sequences. For example, the SARS-CoV-2 polypeptides (e.g., the RBD of SARS-CoV-2 or immunogenic or antigenic fragments thereof) may be fused with the constant domain of immunoglobulins (IgA, IgE, IgG, IgM), or portions thereof (CH1, CH2, CH3, or any combination thereof and portions thereof), or albumin (including but not limited to recombinant human albumin or fragments or variants thereof (see, e.g., U.S. Pat. No. 5,876,969, EP Patent 0 413 622, and U.S. Pat. No. 5,766,883), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with the recombinant vaccinia virus are loaded onto NI²⁺ nitriloacetic acid-agarose column and histidine-tagged proteins can be selectively eluted with imidazole-containing buffers.

In an embodiment, the single-domain antibodies described herein bind SARS-CoV-2 polypeptides and/or the epitope-bearing fragments thereof that are fused with a heterologous antigen (e.g., polypeptide, carbohydrate, phospholipid, or nucleic acid). In specific embodiments, the heterologous antigen is an immunogen.

### Anti-SARS-CoV-2 Single-Domain Antibody Specificity

The binding specificity of single-domain antibodies described herein to SARS-CoV-2 polypeptides, or fragments or variants thereof can be determined by any suitable means. Examples of suitable assays to measure binding specificity include, but are not limited to, immunoprecipitation or in vitro binding assays, such as radioimmunoassay (RIA) or enzyme-linked immunoadsorbent assay (ELISA). Other means, such as, surface plasmon resonance may also be used.

The binding affinity of single-domain antibodies can, for example, be determined by the Scatchard analysis described by Frankel et al., Mol. Immunol., 16:101-106, 1979. In an embodiment, binding affinity is measured by an antigen/antibody dissociation rate. In an embodiment, a high binding affinity is measured by a competition radioimmunoassay. In an embodiment, binding affinity is measured by ELISA. In an embodiment, antibody affinity is measured by flow cytometry.

A single-domain antibody that “specifically binds” or “immunospecifically binds” an antigen (e.g., SARS-CoV-2 or fragments or variants thereof) is a single-domain antibody that binds the antigen with high affinity and does not significantly bind other unrelated antigens.

In an embodiment, the single-domain antibodies described herein bind a SARS-CoV-2 polypeptide or fragment thereof (e.g., the RBD of SARS-CoV-2) with a dissociation constant (K_{d}) of about 50 nM or less. In an embodiment, the single-domain antibodies bind a SARS-CoV-2 polypeptide or fragment thereof with a binding affinity of about 50 nM, about 40 nM, about 30 nM, about 25 nM, about 20 nM, about 15 nM, about 10 nM, about 5 nM, about 4 nM, about 3 nM, about 2 nM, about 1 nM, about 0.5 nM, about 0.25 nM, about 0.1 nM or about 0.05 nM or less.

Some embodiments described herein are directed to a single-domain antibody or fragment thereof fused to one or more Fc regions from an IgG (see, for example, Tables 4 & 5) with a dissociation constant (K_{d}) of about 50 pM or less. In an embodiment, the single-domain antibodies bind a SARS-CoV-2 polypeptide or fragment thereof with a binding affinity of about 50 pM, about 40 pM, about 30 pM, about 20 pM, about 25 pM, about 20 pM, about 10 pM, about 5 pM, about 4 pM, about 3 pM, about 2 pM, about 1 pM, about 0.5 pM, about 0.25 pM, about 0.1 pM or about 0.05 pM or less.

Some embodiments described herein are directed to single-domain antibodies that bind polypeptides comprising, or alternatively consisting of, a polypeptide having an amino acid sequence at least 80%, 85%, 90%, 91%, 92%, 93%, or 94% identical and more preferably at least 95%, 96%, 97%, 98%, 99% or 100% identical to an coronavirus RBD polypeptide having an amino acid sequence of any one of SEQ ID NO: 124, 125, 126, 127, or a combination thereof.

Additional embodiments described herein are directed to single-domain antibodies that bind polypeptides comprising, or alternatively consisting of, a polypeptide having an amino acid sequence of about 90% to 99% sequence identity to a coronavirus RBD polypeptide having the amino acid sequence of any one of SEQ ID NO: 124, 125, 126, 127, or a combination thereof. The single-domain antibodies described herein may selectively bind to a polypeptide having at least about 91%, 92%, 93%, 94%, 95%, 96%, 97%, 98%, 99%, or 100% sequence identity to a coronavirus RBD polypeptide having the amino acid sequence of any one of SEQ ID NO: 124, 125, 126, 127, or a combination thereof.

Additional embodiments described herein are directed to single-domain antibodies that bind polypeptides comprising, or alternatively consisting of, a polypeptide having an amino acid sequence of about 90% to 99% sequence identity to a SARS-CoV-2 Spike polypeptide having the amino acid sequence of SEQ ID NO: 128. The single-domain antibodies described herein may selectively bind to a polypeptide having at least about 91%, 92%, 93%, 94%, 95%, 96%, 97%, 98%, 99%, or 100% sequence identity to a SARS-CoV-2 Spike polypeptide having the amino acid sequence of SEQ ID NO: 128. In an embodiment, the single-domain antibodies described herein may selectively bind to a polypeptide of SEQ ID NO: 128 comprising one or more mutations, such as, for example one or more mutations selected from: R683G, K417N, E484K, and/or N510Y. In an embodiment, the single-domain antibodies described herein may selectively bind to a polypeptide of SEQ ID NO: 128 comprising the following mutations: K417N, E484K, and N510Y.

Single-domain antibodies described herein may bind fragments, variants, derivatives or analogs of a polypeptide of any one of SEQ ID NO: 124, 125, 126, 127, 128, or a combination thereof, such as (i) polypeptides in which one or more of the amino acid residues are substituted with a conserved or non-conserved amino acid residue (preferably a conserved amino acid residue) and such substituted amino acid residue may or may not be one encoded by the genetic code, or (ii) polypeptides in which one or more of the amino acid residues includes a substituent group, or (iii) polypeptides in which the polypeptide is fused with another compound, such as a compound to increase the half-life of the polypeptide (for example, polyethylene glycol), or (iv) polypeptides in which the additional amino acids are fused to the polypeptide, such as an IgG Fc fusion region peptide or leader or secretory sequence or a sequence which is employed for purification of the polypeptide or a proprotein sequence.

Amino acids in the coronavirus polypeptides (e.g., coronavirus RBDs, e.g., SARS-CoV-2 RBD) that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for functional activity, such ligand binding. Accordingly, single-domain antibodies described herein may bind amino acids in the coronavirus polypeptides that are essential for function. In an embodiment, single-domain antibodies described herein bind amino acids in the SARS-CoV-2 polypeptides that are essential for infection, for example by interfering with binding of the RBD of SARS-CoV-2 with ACE2. In an embodiment, single-domain antibodies described herein bind amino acids in the SARS-CoV-2 polypeptides that inhibit or reduce infection, for example by interfering with binding of the RBD of SARS-CoV-2 with ACE2. Sites that are critical for ligand-receptor binding can be determined, for example, by structural analysis such as crystallization, nuclear magnetic resonance or photoaffinity labelling (Smith et al., J. Mal. Biol. 224:899-904 (1992) and de Vos et al. Science 255:306-312 (1992)).

### Anti-SARS-CoV-2 Single-Domain Antibody Activity

The single-domain antibodies described herein neutralize one or more coronaviruses. For example, the single-domain antibodies described herein may neutralize SARS-CoV-1, MERS-CoV, SARS-CoV-2, HCoV-OC43, HCoV-HKU1, HCoV-NL63, HCoV-229E or a combination thereof. The antibodies described herein may neutralize coronaviruses infecting any animal, preferably from a mammal. Most preferably, the antibodies described herein neutralize coronaviruses infecting a human.

In an embodiment, the single-domain antibodies described herein bind to the RBD of one or more coronaviruses, thereby neutralizing infectivity of said one or more coronaviruses. For example, the single-domain antibodies described herein preferably binds the RBD of SARS-CoV-2 and inhibit RBD binding to ACE2, thereby resulting in inhibition of membrane fusion and the entry of the virus into the host cell. In an embodiment, the single-domain antibodies described herein binds the RBD of wild-type SARS-CoV-2 and/or the RBD of a SARS-CoV-2 variant and inhibits RBD binding to ACE2, thereby resulting in inhibition of membrane fusion and the entry of the virus into the host cell. In an embodiment, the SARS-CoV-2 variant comprises one or more mutations in Spike protein, such as, for example at position(s) corresponding to R683, K417, E484, and/or N501 in SEQ ID NO: 128. In an embodiment, the SARS-CoV-2 variant comprises one or more mutations in a Spike protein corresponding to SEQ ID NO: 128 selected from: R683G, K417N, E484K, or N501Y. In an embodiment, the SARS-CoV-2 variant comprises the following mutations in a Spike protein corresponding to SEQ ID NO: 128:K417N, E484K, and N501Y.

Any suitable method may be used to measure neutralization of coronaviruses, such as SARS-CoV-2. For example, an in vitro neutralization assay in which lentiviral particles pseudotyped with SARS-CoV-2 spike protein or an RBD thereof may be used to measure neutralization of single-domain antibodies described herein. Such assays are well-known in the art. Generally, cells are seeded in cell culture plates and incubated for a suitable period of time (e.g., 24-48 hrs) in the presence of a predetermined number of units of a selected coronavirus or pseudotyped virus plus various concentrations of the candidate single-domain antibody. The candidate single-domain antibody that inhibits coronavirus infectivity will inhibit more coronavirus infectivity than the baseline level of coronavirus infectivity measured in the presence of an equivalent concentration of control antibody.

Another suitable method to measure SARS-CoV-2 neutralization is, for example, an in vitro receptor-binding assay to measure the ability of single-domain antibodies described herein to prevent binding of SARS-CoV-2 RBD to its host cell receptor, ACE2.

Other suitable assays will be apparent to the skilled artisan.

Optionally, the candidate single-domain antibody that neutralizes a coronavirus, e.g., SARS-CoV-2, will inhibit at least and/or about 30%, or at least and/or about 40%, or at least and/or about 50%, or at least and/or about 60%, or at least and/or about 70%, or at least and/or about 80%, or at least and/or about 90%, or at least and/or about 95%, or at least and/or about 96%, or at least and/or about 97%, or at least and/or about 98%, or at least and/or about 99%, or about 100% of the infectivity of the coronavirus in a neutralization assay as compared to baseline infectivity measured in the presence of an equivalent concentration of control antibody.

If a single-domain antibody that binds to a particular coronavirus RBD determinant(s) is desired, the candidate antibody can be screened for the presence or absence of differential affinity to wild-type coronavirus RBD and to mutant coronavirus RBD that contains Ala substitution(s) at the determinant(s) of interest as described above. In one aspect, the candidate single-domain antibody can be tested for binding to wild-type coronavirus RBD and mutant coronavirus RBD in an immunoprecipitation or immunoadsorption assay. For example, a capture ELISA can be used wherein plates are coated with a given concentration of wild-type coronavirus RBD or an equal concentration of mutant coronavirus RBD, the coated plates are contacted with equal concentrations of the candidate single-domain antibody, and the bound single-domain antibody is detected enzymatically, e.g., contacting the bound single-domain antibody with HRP-conjugated anti-Ig antibody and developing the HRP color reaction. The candidate antibody that binds to the particular coronavirus RBD determinant(s) of interest will exhibit binding activity with wild-type coronavirus RBD that is greater than the candidate antibody's binding activity with the corresponding Ala-substituted coronavirus RBD mutant (e.g., a binding level with wild-type coronavirus RBD that is above the background binding level with mutant coronavirus RBD). Optionally, the candidate single-domain antibody that binds to the particular coronavirus RBD determinant(s) of interest will exhibit binding activity with the corresponding Ala-substituted coronavirus RBD mutant that is less than about 50%, or less than about 30%, or less than about 20%, or less than about 10%, or less than about 7%, or less than about 6%, or less than about 5%, or less than about 4%, or less than about 3%, or less than about 2%, or less than about 1%, or about 0% of the antibody's binding activity with wild-type coronavirus RBD, e.g., as determined by dividing the HRP color reaction optical density observed for capture ELISA with coronavirus RBD mutant adsorbent by the HRP color reaction optical density observed for capture ELISA with wild-type coronavirus RBD adsorbent.

The single-domain antibodies described herein may possess combinations of coronavirus activity inhibition and coronavirus RBD determinant binding properties described herein. Single-domain antibodies corresponding to these embodiments can be obtained by using combinations of coronavirus competitive binding and/or activity inhibition assays described herein for selection of single-domain antibodies with coronavirus inhibiting properties and the immunoprecipitation or immunoadsorption screening procedures described herein for selection of single-domain antibodies with distinct coronavirus RBD determinant binding properties.

### Anti-SARS-CoV-2 Single-Domain Antibody Mimics

Further, the single-domain antibodies described herein can, in turn, be utilized to generate single-domain antibodies that “mimic” coronavirus RBD polypeptides (e.g., SARS-CoV-2 RBD using techniques well known to those skilled in the art. (See, e.g., Greenspan & Bona, FASEB J. 7(5):437-444 (1993); and Nissinoff, J. Immunol. 147(8):2429-2438 (1991)). For example, single-domain antibodies described herein which bind to a coronavirus RBD and competitively inhibit the binding of a coronavirus to a cell (as determined by assays well known in the art for example, that disclosed, infra) can be used to generate anti-idiotypes that “mimic” a coronavirus RBD to ACE2 and, as a consequence, bind to and neutralize coronaviruses. Such neutralizing anti-idiotypes (including molecules comprising, or alternatively consisting of, single-domain antibody fragments or variants, such as Fab fragments of such anti-idiotypes) can be used in therapeutic regimens to neutralize coronaviruses. For example, such antiidiotypic antibodies can be used to bind coronavirus RBD, and thereby block coronaviruses from binding cells, e.g., ACE2.

### Anti-SARS-CoV-2 Single-Domain Antibody Cross-Reactivity

Single-domain antibodies described herein may also be described or specified in terms of their cross-reactivity. Single-domain antibodies that do not bind any other analog, ortholog, or homolog of a polypeptide described herein are included. Single-domain antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide described herein are also included in the present disclosure.

Single-domain antibodies described herein may cross-react with other coronaviruses and the corresponding epitopes thereof. Single-domain antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide described herein are also included in the present disclosure. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present disclosure are single-domain antibodies that bind polypeptides encoded by polynucleotides that hybridize to a polynucleotide described herein under hybridization conditions (as described herein).

In an embodiment, the single-domain antibodies described herein (including molecules comprising, or alternatively consisting of, antibody fragments of variants thereof), immunospecifically binds to one or more SARS-CoV-2 variants including, but not limited to, variants comprising a mutation in Spike protein at one or more of the following positions: R683, K417, E484, N501. In an embodiment, the single-domain antibodies described herein (including molecules comprising, or alternatively consisting of, antibody fragments of variants thereof), immunospecifically binds to one or more SARS-CoV-2 variants including, but not limited to, variants comprising one or more of the following mutations in Spike protein: R683G, K417N, E484K, and/or N501Y. In an embodiments, the single-domain antibodies described herein (including molecules comprising, or alternatively consisting of, antibody fragments of variants thereof), immunospecifically binds to one or more SARS-CoV-2 variants including, but not limited to, variants comprising the following mutations in Spike protein: K417N, E484K, and N501Y.

In an embodiment, the single-domain antibodies described herein (including molecules comprising, or alternatively consisting of, antibody fragments or variants thereof), immunospecifically bind to SARS-CoV-2 and do not cross-react with any other antigens.

### Variants and Derivatives

As also described above, the present disclosure also provides single-domain antibodies that comprise, or alternatively consist of variants (including derivatives) of the VHH domains and CDRs described herein, which single-domain antibodies immunospecifically bind to SARS-CoV-2. Standard techniques known to those of skill in the art can be used to introduce mutations in the nucleotide sequence encoding a molecule described herein, including, for example, site-directed mutagenesis and PCR-mediated mutagenesis, which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VHH domain, CDR1, CDR2, or CDR3. In an embodiment, the variants have conservative amino acid substitutions at one or more predicted non-essential amino acid residues. A “conservative amino acid substitution” is defined above and generally is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind SARS-CoV-2). Following mutagenesis, the encoded protein may routinely be expressed and the functional and/or biological activity of the encoded protein, (e.g., ability to immunospecifically bind SARS-CoV-2) can be determined using techniques described herein or by routinely modifying techniques known in the art.

The single-domain antibodies described herein include derivatives (e.g., variants) that are modified, e.g., by the covalent attachment of any type of molecule to the single-domain antibody such that covalent attachment does not affect the ability of the antibody to immunospecifically bind to SARS-CoV-2. For example, but not by way of limitation, derivatives described herein include single-domain antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may comprise one or more non-classical amino acids.

### Anti-SARS-CoV-2 Single-Domain Antibodies Sequences and Structure

In a specific embodiment, a single-domain antibody described herein (including a molecule comprising, or alternatively consisting of, an antibody fragment or variant thereof), that immunospecifically binds SARS-CoV-2, comprises, or alternatively consists of, an amino acid sequence encoded by a nucleotide sequence that hybridizes to a nucleotide sequence that is complementary to that encoding one of the VHH domains disclosed herein under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). In an embodiment, a single-domain antibody described herein that immunospecifically binds to SARS-CoV-2, comprises, or alternatively consists of, an amino acid sequence encoded by a nucleotide sequence that hybridizes to a nucleotide sequence that is complementary to that encoding one of the CDRs disclosed herein under stringent conditions, e.g., hybridization under conditions as described above, or under other stringent hybridization conditions which are known to those of skill in the art. In an embodiment, a single-domain antibody described herein that immunospecifically binds to SARS-CoV-2, comprises, or alternatively consists of, an amino acid sequence encoded by a nucleotide sequence that hybridizes to a nucleotide sequence that is complementary to that encoding one of the CDR3s disclosed herein under stringent conditions e.g., hybridization under conditions as described above, or under other stringent hybridization conditions which are known to those of skill in the art. Nucleic acid molecules encoding these single-domain antibodies are also provided by the present disclosure.

The present disclosure also provides single-domain antibodies (including molecules comprising, or alternatively consisting of, antibody fragments or variants thereof) that have one or more of the same biological characteristics as one or more of the single-domain antibodies described herein. By “biological characteristics” is meant, the in vitro or in vivo activities or properties of the single-domain antibodies, for example, the ability to bind to SARS-CoV-2 (e.g., the RBD of SARS-CoV-2 and/or an antigenic and/or epitope region of SARS-CoV-2), the ability to substantially block SARS-CoV-2/ACE2 binding, or the ability to block SARS-CoV-2 infectivity. Optionally, the single-domain antibodies described herein will bind to the same epitope as at least one of the single-domain antibodies specifically referred to herein. Such epitope binding can be routinely determined using assays known in the art.

The present disclosure also provides for single-domain antibodies (including molecules comprising, or alternatively consisting of, antibody fragments or variants thereof), that neutralize SARS-CoV-2 or a variant or fragment thereof, said single-domain antibodies comprising, or alternatively consisting of, a portion (e.g., a VHH domain, CDR1, CDR2, or CDR3) having an amino acid sequence contained within any of SEQ ID NOS: 1-96 and 112-123, or having an amino acid sequence contained within a polypeptide encoded by any of SEQ ID NOS: 97-111, or a fragment or variant thereof as further defined hereinabove.

In other embodiments, the present disclosure provides single-domain antibodies that competitively inhibit binding of a single-domain antibody comprising a fragment (e.g., VHH domain, CDR1, CDR2, or CDR3) described herein or variant thereof to an SARS-CoV-2 polypeptide. In preferred embodiments, the present disclosure provides single-domain antibodies which reduce the binding of a single-domain antibody comprising a fragment (e.g., VHH domain, CDR1, CDR2, or CDR3) described herein or variant thereof to a SARS-CoV-2 polypeptide by between 1% and 10% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 10% and up to 20% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 20% and up to 30% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 30% and up to 40% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 40% and up to 50% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 50% and up to 60% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 60% and up to 70% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 70% and up to 80% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 80% and up to 90% in a competitive inhibition assay.

In preferred embodiments, the present disclosure provides single-domain antibodies that reduce the binding of a single-domain antibody comprising a fragment (e.g., VHH domain, CDR1, CDR2, or CDR3) described herein or variant thereof to a SARS-CoV-2 polypeptide by at least 90% and up to 100% in a competitive inhibition assay.

The present disclosure also provides for mixtures of single-domain antibodies (including molecules comprising, or alternatively consisting of, antibody fragments or variants thereof) that immunospecifically bind to SARS-CoV-2, wherein the mixture has at least one, two, three, four, five or more different single-domain antibodies described herein. In particular, the present disclosure provides for mixtures of different single-domain antibodies that immunospecifically bind to the RBD of SARS-CoV-2. In an embodiment, the present disclosure provides mixtures of at least 2, preferably at least 4, at least 6, at least 8, at least 10, at least 12, at least 15, at least 20, or at least 25 different single-domain antibodies that immunospecifically bind to SARS-CoV-2 wherein at least 1, at least 2, at least 4, at least 6, or at least 10, single-domain antibodies of the mixture is a single-domain antibody described herein. In an embodiment, each single-domain antibody of the mixture is a single-domain antibody described herein.

### Panels of Single-Domain Antibodies

The present disclosure also provides for panels of single-domain antibodies (including molecules comprising, or alternatively consisting of, antibody fragments or variants thereof) that immunospecifically bind to SARS-CoV-2, wherein the panel has at least one, two, three, four, five or more different single-domain antibodies described herein. In particular, the present disclosure provides for panels of different single-domain antibodies that immunospecifically bind to the RBD of SARS-CoV-2. In an embodiment, the present disclosure provides for panels of single-domain antibodies that have different affinities for SARS-CoV-2, different specificities for SARS-CoV-2, or different dissociation rates. The present disclosure provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, single-domain antibodies. Panels of single-domain antibodies can be used, for example, in 96-well plates for assays such as ELISAs.

### Compositions

The present disclosure further provides for compositions comprising, one or more single-domain antibodies (including molecules comprising, or alternatively consisting of antibody fragments or variants described herein). In one embodiment, a composition described herein comprises, one, two, three, four, five, or more single-domain antibodies that comprise or alternatively consist of, a polypeptide having an amino acid sequence of any one or more of the VHH domains contained within any of SEQ ID NOS: 1-15 or a variant thereof. In an embodiment, a composition described herein comprises, one, two, three, four, five, or more single-domain antibodies that comprise, or alternatively consist of, a polypeptide having an amino acid sequence of any one or more of the CDR1 s contained within any of SEQ ID NOS: 1-15, such as any of SEQ ID NOS: 27-40 or a variant thereof. In an embodiment, a composition described herein comprises, one, two, three, four, five or more single-domain antibodies that comprise, or alternatively consist of, a polypeptide having an amino acid sequence of any one or more of the CDR2s contained within any of SEQ ID NOS: 1-15, such as any of SEQ ID NOS: 49-62, or a variant thereof. In a preferred embodiment, a composition described herein comprises, one, two, three, four, five, or more single-domain antibodies that comprise, or alternatively consist of, a polypeptide having an amino acid sequence of any one or more of the CDR3s contained within any of SEQ ID NOS: 1-15, such as any of SEQ ID NOS: 77-91, or a variant thereof.

As discussed in more detail below, a composition described herein may be used either alone or in combination with other compositions. The single-domain antibodies (including molecules comprising, or alternatively consisting of antibody fragments or variants described herein) may further be recombinantly fused to a heterologous polypeptide at the N- or C-terminus or chemically conjugated (including covalently and non-covalently conjugations) to polypeptides or other compositions. For example, single-domain antibodies described herein may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The composition described herein may be a pharmaceutical composition. The composition, including pharmaceutical compositions, may comprise a single-domain antibody or antigen-binding fragment described herein and an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

The single-domain antibodies described herein may be formulated for administration by inhalation, preferably intranasal administration. The single-domain antibodies described herein may be lyophilized, preferably stabilized, for administration by inhalation, preferably intranasal.

Single-domain antibodies described herein (including molecules comprising, or alternatively consisting of antibody fragments or variants described herein) may be used, for example, but not limited to, to detect SARS-CoV-2, including both in vitro and in vivo diagnostic and therapeutic methods. For example, the single-domain antibodies have use in immunoassays for qualitatively and quantitatively measuring levels of SARS-CoV-2 in biological samples. See, e.g., Harlow et al., Antibodies: A Laboratory Manual, (Cold Spring Harbor Laboratory Press, 2nd ed. 1988).

### Nucleic Acids, Vectors, and Host Cells

The present disclosure also provides for an isolated nucleic acid molecule encoding a single-domain antibody described herein (including molecules comprising, or alternatively consisting of, antibody fragments or variants thereof).

Nucleic acid molecules that encode the single-domain antibodies of the present disclosure are described herein. The nucleic acids may be present in whole cells, in a cell lysate, or in a partially purified or substantially pure form. A nucleic acid may be isolated by purification away from other cellular components or other contaminants (e.g., other cellular nucleic acids or proteins) by standard techniques, including alkaline/SDS treatment, CsCl banding, column chromatography, agarose gel electrophoresis and others well known in the art. See Ausubel, et al. (2011) Current Protocols in Molecular Biology John Wiley & Sons, Inc. A nucleic acid described herein may be, for example, DNA or RNA and may or may not comprise intronic sequences. The nucleic acid may be a cDNA molecule. Nucleic acids described herein may be obtained using standard molecular biology techniques. For single-domain antibodies expressed by hybridomas (e.g., hybridomas prepared from transgenic mice carrying camelid immunoglobulin genes as described further herein), cDNAs encoding the heavy chain antibody made by the hybridoma may be obtained by standard PCR amplification or cDNA cloning techniques. For single-domain antibodies obtained from an immunoglobulin gene library (e.g., using phage display techniques), nucleic acid encoding the single-domain antibody may be recovered from the library. Specifically, degenerate codon substitutions may be achieved by generating, e.g., sequences in which the third position of one or more selected codons is substituted with mixed-base and/or deoxyinosine residues. Batzer, et al. (1991) Nucleic Acid Res. 19:5081; Ohtsuka, et al. (1985) J. Biol. Chem. 260: 2605-08; Rossolini, et al. (1994) Mol. Cell. Probes 8: 91-98. A nucleic acid described herein may also be synthetic, for example, DNA with codon usage that has been adapted for expression in the intended host cell or host organism.

The nucleic acid described herein may also be in the form of, be present in and/or be part of a vector, such as for example a plasmid, cosmid or YAC, which again may be in essentially isolated form.

The nucleic acids described herein can be prepared or obtained by any suitable manner based on the information on the amino acid sequences for the polypeptides described herein provided herein, and/or can be isolated from a suitable natural source. To provide analogs, nucleotide sequences encoding naturally occurring VHH domains can, for example, be subjected to site-directed mutagenesis to provide a nucleic acid described herein encoding said analog. Also, several nucleotide sequences, such as at least one nucleotide sequence encoding a single-domain antibody and, for example, nucleic acids encoding one or more linkers, can be linked together in a suitable manner to provide a nucleic acid described herein.

Techniques for generating the nucleic acids described herein will be clear to the skilled person and may include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g., to create cassettes and/or regions that may easily be digested and/or ligated using suitable restriction enzymes), and/or the introduction of mutations by means of a PCR reaction using one or more “mismatched” primers, using for example a sequence of a naturally occurring form of a VHH domain sequence as a template. These and other techniques will be clear to the skilled person.

The nucleic acid described herein may also be in the form of, be present in, and/or be part of a genetic construct. Such genetic constructs generally comprise at least one nucleic acid described herein that is optionally linked to one or more elements of genetic constructs known per se, for example, one or more suitable regulatory elements (e.g., a suitable promoter(s), enhancer(s), terminator(s), etc.) and further elements of genetic constructs referred to herein. Such genetic constructs comprising at least one nucleic acid described herein will also be referred to herein as “genetic constructs described herein”.

The genetic constructs described herein may be DNA or RNA, and are preferably double-stranded DNA. The genetic constructs described herein may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs described herein may be in the form of a vector, such as for example a plasmid, cosmid, YAC, a viral vector or transposon. In particular, the vector may be an expression vector, e.g., a vector that can provide for expression in vitro and/or in vivo (e.g., in a suitable host cell, host organism and/or expression system).

In a non-limiting aspect, a genetic construct described herein comprises
i) at least one nucleic acid described herein; operably connected to
ii) one or more regulatory elements, such as a promoter and optionally a suitable terminator; and optionally also
iii) one or more further elements of genetic constructs known per se.

The nucleic acids described herein and/or the genetic constructs described herein may be used to transform a host cell or host organism, e.g., for expression and/or production of the amino acid sequence, single-domain antibody or polypeptide described herein. Suitable hosts or host cells will be clear may, for example, be any suitable fungal, prokaryotic or eukaryotic cell, or cell line or any suitable fungal, prokaryotic or eukaryotic organism; as well as all other hosts or host cells known for the expression and production of antibodies and antibody fragments (including but not limited to (single) domain antibodies and scFv fragments), which will be clear to the skilled person.

The amino acid sequences, single-domain antibodies, and polypeptides described herein can also be introduced and expressed in one or more cells, tissues or organs of a multicellular organism, for example for prophylactic and/or therapeutic purposes (e.g., as a gene therapy).

For expression of the single-domain antibodies in a cell, they may also be expressed as “intrabodies”, for example, as described in WO 94/02610, WO 95/22618 and U.S. Pat. No. 7,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

The amino acid sequences, single-domain antibodies and polypeptides described herein can, for example, also be produced in the milk of transgenic mammals, for example in the milk of rabbits, cows, goats or sheep (see, e.g., U.S. Pat. Nos. 6,741,957, 6,304,489 and 6,849,992 for general techniques for introducing transgenes into mammals), in plants or parts of plants including but not limited to their leaves, flowers, fruits, seed, roots or tubers (e.g., in tobacco, maize, soybean or alfalfa) or in for example pupae of the silkworm Bombix mori.

Furthermore, the amino acid sequences, single-domain antibodies, and polypeptides described herein can also be expressed and/or produced in cell-free expression systems, and suitable examples of such systems will be clear to the skilled person. Some preferred, but non-limiting, examples include expression in the wheat germ system; in rabbit reticulocyte lysates; or in the E. coli Zubay system.

As mentioned above, one of the advantages of the use of single-domain antibodies is that the polypeptides based thereon can be prepared through expression in a suitable bacterial system, and suitable bacterial expression systems, vectors, host cells, regulatory elements, etc., will be clear to the skilled person. It should however be noted that the present disclosure in its broadest sense is not limited to expression in bacterial systems.

Preferably, an in vivo or in vitro expression system, such as a bacterial expression system, is used that provides the polypeptides described herein in a form that is suitable for pharmaceutical use, and such expression systems will again be clear to the skilled person. As also will be clear to the skilled person, polypeptides described herein suitable for pharmaceutical use can be prepared using techniques for peptide synthesis.

For production on industrial scale, preferred heterologous hosts for the (industrial) production of single-domain antibodies or single-domain antibody-containing protein therapeutics include strains of E. coli, Pichia pastoris, S. cerevisiae that are suitable for large scale expression/production/fermentation, and in particular for large scale pharmaceutical (e.g., GMP grade) expression/production/fermentation. Suitable examples of such strains will be clear to the skilled person.

Alternatively, mammalian cell lines, in particular Chinese hamster ovary (CHO) cells, can be used for large scale expression/production/fermentation, and in particular for large scale pharmaceutical expression/production/fermentation.

The choice of the specific expression system depends, in part, on the requirement for certain post-translational modifications, more specifically glycosylation. The production of a single-domain antibody-containing recombinant protein for which glycosylation is desired or required would necessitate the use of mammalian expression hosts that have the ability to glycosylate the expressed protein. In this respect, it will be clear to the skilled person that the glycosylation pattern obtained (e.g., the kind, number and position of residues attached) will depend on the cell or cell line that is used for expression. Preferably, either a human cell or cell line is used (e.g., leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the present disclosure, depending on the desired amino acid sequence, single-domain antibody or polypeptide to be obtained.

Thus, the amino acid sequence, single-domain antibody, or polypeptide described herein may be glycosylated. The amino acid sequence, single-domain antibody, or polypeptide described herein may be non-glycosylated.

The amino acid sequence, single-domain antibody or polypeptide described herein may be produced in a bacterial cell, in particular a bacterial cell suitable for large scale pharmaceutical production, such as cells of the strains mentioned above.

The amino acid sequence, single-domain antibody, or polypeptide described herein may be produced in a yeast cell, in particular a yeast cell suitable for large scale pharmaceutical production, such as cells of the species mentioned above.

The amino acid sequence, single-domain antibody, or polypeptide described herein may be produced in a mammalian cell, in particular in a human cell or in a cell of a human cell line, and more in particular in a human cell or in a cell of a human cell line that is suitable for large scale pharmaceutical production, such as the cell lines described herein above.

When expression in a host cell is used to produce the amino acid sequences, single-domain antibodies, and the polypeptides described herein, the amino acid sequences, single-domain antibodies and polypeptides described herein can be produced either intracellullarly (e.g., in the cytosol, in the periplasma or in inclusion bodies) and then isolated from the host cells and optionally further purified; or can be produced extracellularly (e.g., in the medium in which the host cells are cultured) and then isolated from the culture medium and optionally further purified. Thus, according to one non-limiting aspect, the amino acid sequence, single-domain antibody or polypeptide described herein is an amino acid sequence, single-domain antibody or polypeptide that has been produced intracellularly and that has been isolated from the host cell, and in particular from a bacterial cell or from an inclusion body in a bacterial cell. The amino acid sequence, single-domain antibodies, or polypeptide described herein may be an amino acid sequence, single-domain antibody or polypeptide that has been produced extracellularly, and that has been isolated from the medium in which the host cell is cultivated.

Suitable techniques for transforming a host or host cell described herein will be clear to the skilled person and may depend on the intended host cell/host organism and the genetic construct to be used. After transformation, a step for detecting and selecting those host cells or host organisms that have been successfully transformed with the nucleotide sequence/genetic construct described herein may be performed. This may, for example, be a selection step based on a selectable marker present in the genetic construct described herein or a step involving the detection of the amino acid sequence described herein, e.g., using specific antibodies. Current Protocols in Molecular Biology (2012) Ausubel et al. John Wiley & Sons, Inc.

The transformed host cell (which may be in the form or a stable cell line) or host organisms (which may be in the form of a stable mutant line or strain) form further aspects described herein.

Preferably, these host cells or host organisms are such that they express, or are capable of expressing (e.g., under suitable conditions), an amino acid sequence, single-domain antibody or polypeptide described herein (and in case of a host organism: in at least one cell, part, tissue or organ thereof). The present disclosure also provides further generations, progeny and/or offspring of the host cell or host organism described herein that may, e.g., be obtained by cell division or by sexual or asexual reproduction.

### Methods for Producing Antibodies

The single-domain antibodies described herein (including other molecules comprising, or alternatively consisting of single-domain antibody fragments or variants described herein) can be produced by any method known in the art for the synthesis of antibodies, in particular, by chemical synthesis or preferably, by recombinant expression techniques.

In an embodiment, the present disclosure relates to a method for generating single-domain antibodies that are directed against SARS-CoV-2. In one aspect, said method at least comprises the steps of:
a) providing a panel, set, collection, or library of single-domain antibody sequences; and
b) screening said panel, set, collection, or library of single-domain antibody sequences for single-domain antibody sequences that can bind to and/or have affinity for SARS-CoV-2; and
c) isolating the single-domain antibody or single-domain antibodies that can bind to and/or have affinity for SARS-CoV-2.

In such a method, the panel, set, collection or library of single-domain antibody sequences may be a naive panel, set, collection, or library of single-domain antibody sequences; a synthetic or semi-synthetic panel, set, collection, or library of single-domain antibody sequences; and/or a panel, set, collection, or library of single-domain antibody sequences that have been subjected to affinity maturation.

In a preferred aspect of this method, the panel, set, collection, or library of single-domain antibody sequences may be an immune panel, set, collection, or library of single-domain antibody sequences, and in particular an immune panel, set, collection, or library of VHH sequences that have been derived from a species of Camelid that has been immunized with SARS-CoV-2 or with a suitable antigenic determinant based thereon or derived therefrom, such as an antigenic part, fragment, region, domain, loop, or other epitope thereof. In one aspect, said antigenic determinant may be the receptor binding domain (RBD) and/or the Spike protein of SARS-CoV-2 or a fragment thereof.

In the above methods, the panel, set, collection, or library of single-domain antibodies or VHH sequences may be displayed on a phage, phagemid, ribosome or suitable microorganism (e.g., yeast), to facilitate screening. Suitable methods, techniques, and host organisms for displaying and screening (a panel, set, collection, or library of) single-domain antibody sequences will be clear to the person skilled in the art, for example on the basis of the further disclosure herein. See also, for example WO 03/054016 and Hoogenboom (Nature Biotechnology, 23(9):1105-1116 (2005)); Basic Methods in Antibody Production and Characterization Howard & Bethell (Eds.) (2000) CRC Press.

In another aspect, the method for generating single-domain antibody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for SARS-CoV-2; and (ii) cells that express heavy chain antibodies, in which substeps (i) and (ii) can be performed essentially as a single screening step or in any suitable order as two separate screening steps, to provide at least one cell that expresses a heavy chain antibody that can bind to and/or has affinity for SARS-CoV-2; and
c) either (i) isolating from said cell the VHH sequence present in said heavy chain antibody; or (ii) isolating from said cell a nucleic acid sequence that encodes the VHH sequence present in said heavy chain antibody, followed by expressing said VHH domain.

In the method according to this aspect, the collection or sample of cells may, for example, be a collection or sample of B cells. Also, in this method, the sample of cells may be derived from a Camelid that has been immunized with SARS-CoV-2 or a suitable antigenic determinant based thereon or derived therefrom, such as an antigenic part, fragment, region, domain, loop, or other epitope thereof. In one aspect, said antigenic determinant may be the RBD and/or the Spike protein of SARS-CoV-2 or a fragment thereof.

The above method may be performed in any suitable manner. Reference is for example made to EP 0 542 810, WO 05/19824, WO 04/051268, WO 04/106377, and WO 06/079372. The screening of step b) is preferably performed using a flow cytometry technique such as FACS. For this, reference is, for example, made to Lieby et al., Blood, Vol. 97, No. 12, 3820.

In another aspect, the method for generating an amino acid sequence directed against SARS-CoV-2 may comprise at least the steps of:
a) providing a panel, set, collection, or library of nucleic acid sequences encoding heavy chain antibodies or VHH sequences;
b) screening said panel, set, collection, or library of nucleic acid sequences for nucleic acid sequences that encode a heavy chain antibody or a VHH sequence that can bind to and/or has affinity for SARS-CoV-2;
c) isolating said nucleic acid sequence, followed by expressing the VHH sequence present in said heavy chain antibody or by expressing said VHH sequence, respectively.

In such a method, the panel, set, collection or library of nucleic acid sequences encoding heavy chain antibodies or VHH sequences may, for example, be a panel, set, collection, or library of nucleic acid sequences encoding a naive panel, set, collection, or library of heavy chain antibodies or VHH sequences; a panel, set, collection or library of nucleic acid sequences encoding a synthetic or semi-synthetic panel, set, collection or library of VHH sequences; and/or a panel, set, collection or library of nucleic acid sequences encoding a panel, set, collection, or library of VHH sequences that have been subjected to affinity maturation.

In a preferred aspect of this method, the panel, set, collection, or library of nucleic acid sequences may be an immune panel, set, collection, or library of nucleic acid sequences encoding heavy chain antibodies or VHH sequences derived from a Camelid that has been immunized with SARS-CoV-2 or with a suitable antigenic determinant based thereon or derived therefrom, such as an antigenic part, fragment, region, domain, loop or other epitope thereof. In one aspect, said antigenic determinant may be the RBD and/or Spike protein of SARS-CoV-2 or fragments thereof.

In the above methods, the panel, set, collection, or library of nucleotide sequences may be displayed on a phage, phagemid, ribosome, or suitable microorganism (e.g., yeast), to facilitate screening. Suitable methods, techniques, and host organisms for displaying and screening (a panel, set, collection, or library of) nucleotide sequences encoding amino acid sequences will be clear to the person skilled in the art, for example, on the basis of the further disclosure herein. See also WO 03/054016 and Hoogenboom (Nature Biotechnology, 23(9): 1105-1116 (2005)).

The screening step of the methods described herein can also be performed as a selection step. Accordingly, the term “screening” as used in the present description can comprise selection, screening, or any suitable combination of selection and/or screening techniques. Also, when a panel, set, collection, or library of sequences is used, it may comprise any suitable number of sequences, such as 1, 2, 3, or about 5, 10, 50, 100, 500, 1000, 5000, 10⁴, 10⁵, 10⁶, 10⁷, 10⁸ or more sequences.

Also, one or more or all of the sequences in the above panel, set, collection, or library of amino acid sequences may be obtained or defined by rational, or semi-empirical approaches such as computer modelling techniques or biostatics or data-mining techniques.

Furthermore, such a panel, set, collection, or library can comprise one, two or more sequences that are variants from one another (e.g., with designed point mutations or with randomized positions), compromise multiple sequences derived from a diverse set of naturally-diversified sequences (e.g., an immune library), or any other source of diverse sequences (as described, for example, in Hoogenboom et al, Nat Biotechnol 23:1105 (2005) and Binz et al, Nat Biotechnol 23:1247 (2005)). Such panel, set, collection, or library of sequences can be displayed on the surface of a phage particle, a ribosome, a bacterium, a yeast cell, a mammalian cell, and linked to the nucleotide sequence encoding the amino acid sequence within these carriers. This makes such panel, set, collection, or library amenable to selection procedures to isolate the desired amino acid sequences described herein. More generally, when a sequence is displayed on a suitable host or host cell, it is also possible to first isolate from said host or host cell a nucleotide sequence that encodes the desired sequence, and then to obtain the desired sequence by expressing said nucleotide sequence in a suitable host organism. Again, this can be performed in any suitable manner.

Yet another technique for obtaining VHH sequences or single-domain antibody sequences directed against SARS-CoV-2 involves immunizing a transgenic mammal that is capable of expressing heavy chain antibodies (e.g., to raise an immune response and/or heavy chain antibodies directed against SARS-CoV-2), obtaining a suitable biological sample from said transgenic mammal that contains (nucleic acid sequences encoding) said VHH sequences or single-domain antibody sequences (e.g., a blood sample, serum sample, or sample of B-cells), and then generating VHH sequences directed against SARS-CoV-2 starting from said sample using any suitable technique (e.g., any of the methods described herein or a hybridoma technique). For example, for this purpose, heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103(41):15130-5 can be used. For example, such heavy chain antibody-expressing mice can express heavy chain antibodies with any suitable single variable domain, such as single variable domains from natural sources (e.g., human single variable domains, Camelid single variable domains or shark single variable domains), as well as, for example, synthetic or semi-synthetic single variable domains.

The present disclosure also relates to the VHH sequences or single-domain antibody sequences that are obtained by the above methods, or alternatively, by a method that comprises the one of the above methods and, in addition, at least the steps of determining the nucleotide sequence or amino acid sequence of said VHH sequence or single-domain antibody sequence; and of expressing or synthesizing said VHH sequence or single-domain antibody sequence in a known manner, such as by expression in a suitable host cell or host organism or by chemical synthesis.

Once a single-domain antibody molecule described herein (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the single-domain antibodies described herein may be fused to heterologous polypeptide sequences described herein or otherwise known in the art, to facilitate purification.

The present disclosure also provides methods for recombinantly producing the anti-SARS-CoV-2 single-domain antibodies described herein. Methods of producing the single-domain antibodies described herein are well known to those of ordinary skill in the art. The anti-SARS-CoV-2 single-domain antibodies described herein may also be produced by constructing, using conventional techniques well known to those of ordinary skill in the art, an expression vector comprising an operon and a DNA sequence encoding the anti-SARS-CoV-2 single-domain antibodies described herein. Furthermore, the present disclosure relates to vectors, especially plasmids, cosmids, viruses, bacteriophages and other vectors common in genetic engineering, which comprise the above-mentioned nucleic acid molecules described herein. The nucleic acid molecules contained in the vectors may be linked to regulatory elements that ensure the transcription in prokaryotic and eukaryotic cells.

Vectors comprise elements that facilitate manipulation for the expression of a foreign protein within the target host cell. Conveniently, manipulation of sequences and production of DNA for transformation is first performed in a bacterial host (e.g., E. coli) and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector comprising the selection gene will not survive in the culture medium. Typical selection genes encode proteins that confer resistance to antibiotics or other toxins, complement auxotrophic deficiencies, or supply critical nutrients not available from complex media. Exemplary vectors and methods for transformation of yeast are described in the art. See, e.g., Burke, et al. (2000) Methods in Yeast Genetics Cold Spring Harbor Laboratory Press; Gold Sprinq Harbor Protocols (2021) Cold Spring Harbor Laboratory Press.

The polynucleotide coding for the anti-SARS-CoV-2 single-domain antibodies may be operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included (e.g., a signal sequence).

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” refers broadly to contiguous linked DNA sequences, and, in the case of a secretory leader, contiguous and in reading frame. However, enhancers do not have to be contiguous.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (e.g., that increase levels of transcription in response to absence of a repressor). Inducible promoters may initiate increased levels of transcription from DNA under their control in response to some change in culture conditions (e.g., the presence or absence of a nutrient or a change in temperature.)

The expression vectors are transfected into a host cell by convention techniques well known to those of ordinary skill in the art to produce a transfected host cell, said transfected host cell cultured by conventional techniques well known to those of ordinary skill in the art to produce said anti-SARS-CoV-2 single-domain antibodies.

The host cells used to express the anti-SARS-CoV-2 single-domain antibodies may be either a bacterial cell such as E. coli, yeast (e.g., S. cerevisiae), or a eukaryotic cell (e.g., a mammalian cell line). A mammalian cell of a well-defined type for this purpose, such as a myeloma cell, 3T3, HeLa, C6A2780, Vero, MOCK II, a Chinese hamster ovary (CHO), Sf9, Sf21, COS, NSO, or HEK293 cell line may be used.

The general methods by which the vectors may be constructed, transfection methods required to produce the host cell and culturing methods required to produce the single-domain antibodies, and fragments thereof, from said host cells all include conventional techniques. Although preferably the cell line used to produce the anti-SARS-CoV-2 single-domain antibodies is a mammalian cell line, any other suitable cell line, such as a bacterial cell line such as an E. coli-derived bacterial strain, or a yeast cell line, may be used.

Similarly, once produced the anti-SARS-CoV-2 single-domain antibodies may be purified according to standard procedures in the art, such as for example cross-flow filtration, ammonium sulphate precipitation, and affinity column chromatography.

### Diagnostic Uses of Anti-SARS-CoV-2 Single-Domain Antibodies

Labeled single-domain antibodies described herein (including molecules comprising, or alternatively consisting of, antibody fragments or variants thereof) which specifically bind to SARS-CoV-2 can be used for diagnostic purposes to detect, diagnose, prognose, or monitor diseases and/or disorders associated with coronavirus infection. The present disclosure provides for the detection of SARS-CoV-2 comprising: (a) assaying the presence of SARS-CoV-2 in a biological sample from a subject using one or more single-domain antibodies described herein that immunospecifically binds to SARS-CoV-2; and (b) comparing the level of SARS-CoV-2 with a control, e.g., in normal biological samples with no known coronavirus infection.

By “biological sample” is intended any fluids and/or cells obtained from a subject, body fluid, body tissue, body cell, cell line, tissue culture, or other source that may comprise SARS-CoV-2 protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

In addition, the anti-SARS-CoV-2 single-domain antibodies described herein are useful in diagnostic assays for the detection of SARS-CoV-2 in cells or tissues wherein the single-domain antibodies are labeled as described below and/or are immobilized on an insoluble matrix. Anti-SARS-CoV-2 single-domain antibodies also are useful for the affinity purification of SARS-CoV-2 polypeptides, such as SARS-CoV-2 RBD polypeptides, from recombinant cell culture or natural sources.

Anti-SARS-CoV-2 single-domain antibodies can be used for the detection of SARS-CoV-2 in any one of a number of well-known diagnostic assay methods. For example, a biological sample may be assayed for SARS-CoV-2 by obtaining the sample from a desired source, admixing the sample with anti-SARS-CoV-2 single-domain antibody to allow the single-domain antibody to form antibody/SARS-CoV-2 complex with any SARS-CoV-2 present in the mixture, and detecting any antibody/SARS-CoV-2 complex present in the mixture. The biological sample may be prepared for assay by methods known in the art that are suitable for the particular sample. The methods of admixing the sample with single-domain antibodies and the methods of detecting antibody/SARS-CoV-2 complex are chosen according to the type of assay used. Such assays include competitive and sandwich assays, and steric inhibition assays. Competitive and sandwich methods employ a phase-separation step as an integral part of the method while steric inhibition assays are conducted in a single reaction mixture.

Analytical methods for SARS-CoV-2 detection use one or more of the following reagents: labeled SARS-CoV-2 analogue, immobilized SARS-CoV-2 analogue, labeled anti-SARS-CoV-2 single-domain antibody, immobilized anti-SARS-CoV-2 single-domain antibody and steric conjugates. The labeled reagents also are known as “tracers.”

The label used is any detectable functionality that does not interfere with the binding of SARS-CoV-2 and anti-SARS-CoV-2 single-domain antibody. Numerous labels are known for use in immunoassay, examples including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the single-domain antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al., Nature, 144:945 (1962); David eta al., Biochemistry, 13: 1014-1021 (1974); Pain et al., J. Immunol. Methods, 40: 219-230 (1981); and Nygren, J. Histochem. and Cytochem., 30: 407-412 (1982). Preferred labels herein are enzymes such as horseradish peroxidase and alkaline phosphatase.

The conjugation of such label, including the enzymes, to the single-domain antibody is a standard manipulative procedure for one of ordinary skill in immunoassay techniques. See, for example, O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Immobilization of reagents is required for certain assay methods. Immobilization entails separating the anti-SARS-CoV-2 single-domain antibody from any SARS-CoV-2 that remains free in solution. This conventionally is accomplished by either insolubilizing the anti-SARS-CoV-2 antibody or SARS-CoV-2 analogue before the assay procedure, as by adsorption to a water-insoluble matrix or surface (Bennich et al., U.S. Pat. No. 3,720,760), by covalent coupling (for example, using glutaraldehyde cross-linking), or by insolubilizing the anti-SARS-CoV-2 antibody or SARS-CoV-2 analogue afterward, e.g., by immunoprecipitation.

Other assay methods, known as competitive or sandwich assays, are well established and widely used in the commercial diagnostics industry.

Competitive assays rely on the ability of a tracer SARS-CoV-2 analogue to compete with the test sample SARS-CoV-2 for a limited number of anti-SARS-CoV-2 single-domain antibody antigen-binding sites. The anti-SARS-CoV-2 single-domain antibody generally is insolubilized before or after the competition and then the tracer and SARS-CoV-2 bound to the anti-SARS-CoV-2 single-domain antibody are separated from the unbound tracer and SARS-CoV-2. This separation is accomplished by decanting (where the binding partner was preinsolubilized) or by centrifuging (where the binding partner was precipitated after the competitive reaction). The amount of test sample SARS-CoV-2 is inversely proportional to the amount of bound tracer as measured by the amount of marker substance. Dose-response curves with known amounts of SARS-CoV-2 are prepared and compared with the test results to quantitatively determine the amount of SARS-CoV-2 present in the test sample. These assays are called ELISA systems when enzymes are used as the detectable markers.

Another species of competitive assay, called a “homogeneous” assay, does not require a phase separation. Here, a conjugate of an enzyme with the SARS-CoV-2 is prepared and used such that when anti-SARS-CoV-2 single-domain antibody binds to the SARS-CoV-2 the presence of the anti-SARS-CoV-2 single-domain antibody modifies the enzyme activity. In this case, the SARS-CoV-2 or its immunologically active fragments are conjugated with a bifunctional organic bridge to an enzyme such as peroxidase. Conjugates are selected for use with anti-SARS-CoV-2 single-domain antibody so that binding of the anti-SARS-CoV-2 single-domain antibody inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small SARS-CoV-2 fragment so that antibody to hapten is substantially unable to bind the conjugate at the same time as anti-SARS-CoV-2 single-domain antibody. Under this assay procedure the SARS-CoV-2 present in the test sample will bind anti-SARS-CoV-2 single-domain antibody, thereby allowing anti-hapten to bind the conjugate, resulting in a change in the character of the conjugate hapten, e.g., a change in fluorescence when the hapten is a fluorophore.

Sandwich assays particularly are useful for the determination of SARS-CoV-2 or anti-SARS-CoV-2 single-domain antibodies. In sequential sandwich assays an immobilized anti-SARS-CoV-2 single-domain antibody is used to adsorb test sample SARS-CoV-2, the test sample is removed as by washing, the bound SARS-CoV-2 is used to adsorb a second, labeled anti-SARS-CoV-2 single-domain antibody and bound material is then separated from residual tracer. The amount of bound tracer is directly proportional to test sample SARS-CoV-2. In “simultaneous” sandwich assays, the test sample is not separated before adding the labeled anti-SARS-CoV-2. A sequential sandwich assay using an anti-SARS-CoV-2 single-domain antibody as one antibody and a polyclonal anti-SARS-CoV-2 antibody as the other is useful in testing samples for SARS-CoV-2.

The foregoing are merely exemplary diagnostic assays for SARS-CoV-2. Other methods now or hereafter developed that use anti-SARS-CoV-2 single-domain antibody for the determination of SARS-CoV-2 are included within the scope hereof, including the bioassays described above.

### Therapeutic Methods

Single-domain antibodies described herein (including molecules comprising, or alternatively consisting of, antibody fragments or variants thereof) which specifically bind to SARS-CoV-2 can be used for diagnostic purposes to detect, diagnose, prognose, or monitor coronavirus infections (preferably SARS-CoV-2 infections), and/or diseases or conditions associated therewith (e.g., COVID-19).

### Therapeutic Compositions and Administration of Anti-SARS-CoV-2 Single-Domain Antibodies

Therapeutic formulations of the anti-SARS-CoV-2 single-domain antibodies described herein are prepared for storage by mixing said antibody having the desired degree of purity with optional physiologically acceptable carriers, excipients, or stabilizers (Remington: The Science and Practice of Pharmacy, 22nd Edition, Alfonso, R., ed, Mack Publishing Co. (Easton, Pa.: 2012)), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The anti-SARS-CoV-2 single-domain antibody to be used for in vivo administration must be sterile. This is readily accomplished by filtration through sterile filtration membranes, prior to or following lyophilization and reconstitution. The anti-SARS-CoV-2 single-domain antibody ordinarily will be stored in lyophilized form or in solution.

Therapeutic anti-SARS-CoV-2 single-domain antibody compositions generally are placed into a container having a sterile access port, for example, an intravenous solution bag or vial having a stopper pierceable by a hypodermic injection needle.

The route of anti-SARS-CoV-2 single-domain antibody administration is in accord with known methods, e.g., injection or infusion by intravenous, intraperitoneal, intracerebral, subcutaneous, intramuscular, intraocular, inhaled, optionally intranasal, intrapulmonary, intraarterial, intracerebrospinal, or intralesional routes, orally, intrathecally, parentally, or by sustained release systems as noted below. Any suitable combination of administration routes may also be used. In an embodiment, the single-domain antibody is given systemically.

Suitable examples of sustained-release preparations include semipermeable polymer matrices in the form of shaped articles, e.g., films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al., Biopolymers, 22: 547-556 (1983)), poly (2-hydroxyethyl-methacrylate) (Langer et al., J. Biomed. Mater. Res., 15: 167-277 (1981) and Langer, Chem. Tech., 12: 98-105 (1982)), ethylene vinyl acetate (Langer et al., supra) or poly-D-(−)-3-hydroxybutyric acid (EP 133,988). Sustained-release anti-SARS-CoV-2 single-domain antibody compositions also include liposomally entrapped antibody. Liposomes comprising antibody are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal antibody therapy.

The anti-SARS-CoV-2 single-domain antibodies described herein may be formulated to be administered via intranasal and/or intrapulmonary routes, for example, via inhalation. Likewise, the anti-SARS-CoV-2 single-domain antibodies described herein may be administered via intranasal and/or intrapulmonary routes, for example, via inhalation.

For example, the anti-SARS-CoV-2 single-domain antibodies and compositions comprising the same described herein may be delivered in the form of vapors, drops, sprays, aerosols, or a powder. Commercially available nebulizers for liquid formulations, including jet nebulizers and ultrasonic nebulizers are useful for administration. Liquid formulations can be directly nebulized and lyophilized powder can be nebulized after reconstitution. Alternatively, anti-SARS-CoV-2 single-domain antibody can be aerosolized using a fluorocarbon formulation and a metered dose inhaler, or inhaled as a lyophilized and milled powder.

The anti-SARS-CoV-2 single-domain antibodies and compositions comprising the same may be delivered by a device configured for inhalation administration. The device configured for inhalation delivery of the single-domain antibody or antigen binding fragment or composition described herein. The device may be configured for inhalation delivery via intranasal, intrapulmonary, or a combination thereof.

The device may be an insufflator, breath actuated inhaler, mechanical powder sprayer, electrically power nebulizer (atomizer), nebulizer, atomizer, gas driven spray systems, gas driven atomixers, or mechanical pump sprays.

An “effective amount” of anti-SARS-CoV-2 single-domain antibody to be employed therapeutically will depend, for example, upon the therapeutic objectives, the route of administration, the type of anti-SARS-CoV-2 single-domain antibody employed, and the condition of the patient. Accordingly, it will be necessary for the clinician to titer the dosage and modify the route of administration as required to obtain the optimal therapeutic effect. The clinician may administer the anti-SARS-CoV-2 single-domain antibody until a dosage is reached that achieves the desired effect. The progress of this therapy is easily monitored by conventional assays.

Anti-SARS-CoV-2 single-domain antibodies described herein can be used to neutralize SARS-CoV-2 and variants thereof so as to prevent or reduce the ability of SARS-CoV-2 to bind to ACE2, thereby preventing or reducing viral entry into host cells. Accordingly, therapeutic administration of anti-SARS-CoV-2 single-domain antibodies described herein may be particularly useful to treat and/or prevent SARS-CoV-2 infection (e.g., COVID-19).

Therapeutic or pharmaceutical compositions comprising the anti-SARS-CoV-2 single-domain antibodies described herein may be used to treat, prevent or ameliorate diagnose or prognose, coronavirus infection (e.g., SARS-CoV-2 infection) and/or medical conditions associated therewith (e.g., COVID-19, asthma, acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof).

Therapeutic or pharmaceutical compositions comprising the anti-SARS-CoV-2 single-domain antibodies described herein may be administered to an animal (preferably, a mammal; more preferably, a human) to treat, prevent or ameliorate coronavirus infections and/or conditions associated with coronavirus infections. Examples of conditions associated with coronavirus infections include, but are not limited to, COVID-19, asthma, acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

As a general proposition, the initial pharmaceutically effective amount of the single-domain antibody administered parenterally will be in the range of about 0.1 to 500 mg/kg of patient body weight per day, with the typical initial range of single-domain antibody used being 0.2 to 100 mg/kg/day, more preferably 0.3 to 20 mg/kg/day. In an embodiment, the effective amount of the single-domain antibody is between about 1 ng and 1,000 ng, between about 1 pg and 1,000 pg, between about 1 mg and 1,000 mg, or between about 1 g and 1,000 g. The desired dosage can be delivered by a single bolus administration, by multiple bolus administrations, or by continuous infusion administration of single-domain antibody, depending on the pattern of pharmacokinetic decay that the practitioner wishes to achieve. As a non-limiting example, the single-domain antibody can be administered at least 1, 2, 3, 4, 5, 6, 7, 8, 9, or 10 times. In an embodiment, the single-domain antibody or antigen binding fragment thereof is administered over the course of 1, 2, 3, 4, 5, 6, or 7 days. In another embodiment, the single-domain antibody or antigen binding fragment thereof is administered over the course of 1, 2, 3, or 4 weeks.

The single-domain antibody need not be, but is optionally, formulated with one or more agents currently used to prevent or treat coronavirus infection or the condition associated with coronavirus infection in question. The effective amount of such other agents depends on the amount of anti-SARS-CoV-2 single-domain antibody present in the formulation, the type of disorder or treatment, and other factors discussed above. These are generally used in the same dosages and with administration routes as used hereinbefore or about from 1 to 99% of the heretofore employed dosages.

### Kits

A pharmaceutical pack or kit may comprise one or more containers filled with one or more of the ingredients of the pharmaceutical compositions comprising the anti-SARS-CoV-2 single-domain antibodies described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits are provided that can be used in the methods described herein. A kit may comprise a single-domain antibody described herein, preferably a purified single-domain antibody, in one or more containers. In an alternative embodiment, a kit may comprise an antibody fragment that immunospecifically binds to SARS-CoV-2. Kits may comprise a substantially isolated SARS-CoV-2 polypeptide as a control.

Kits may further comprise a control antibody that does not react with SARS-CoV-2. In another specific embodiment, the kits described herein comprise a means for detecting the binding of an antibody to SARS-CoV-2 (e.g., the single-domain antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized SARS-CoV-2 polypeptide. The SARS-CoV-2 polypeptide provided in the kit may also be attached to a solid support. In a more specific embodiment, the detecting means of the above-described kit includes a solid support to which SARS-CoV-2 polypeptide is attached. Such a kit may also include a non-attached reporter-labeled anti-human antibody. In this embodiment, binding of the single-domain antibody to SARS-CoV-2 can be detected by binding of the said reporter-labeled antibody.

A diagnostic kit for use in screening a biological sample comprising antigens of the polypeptide described herein. The diagnostic kit includes a substantially isolated single-domain antibody specifically immunoreactive with SARS-CoV-2, and means for detecting the binding of SARS-CoV-2 to the single-domain antibody. In one embodiment, the single-domain antibody is attached to a solid support. The detecting means of the kit may include a second, labeled antibody. Alternatively, or in addition, the detecting means may include a labeled, competing antigen.

In one diagnostic configuration, a biological sample is reacted with a solid phase reagent having a surface-bound SARS-CoV-2 polypeptide obtained by the methods described herein. After SARS-CoV-2 binds to a specific single-domain antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-SARS-CoV-2 single-domain antibody on the solid support. The reporter may be an enzyme, which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, through a free amine group, to a chemically reactive group on the solid support, such as an activated carboxyl, hydroxyl, or aldehyde group. Alternatively, streptavidin coated plates can be used in conjunction with biotinylated antigen(s).

Thus, the present disclosure provides an assay system or kit for carrying out this diagnostic method. The kit generally includes a support with surface-bound recombinant SARS-CoV-2 polypeptide, and a reporter-labeled anti-human antibody for detecting surface-bound anti-SARS-CoV-2 single-domain antibody.

Further details described herein can be found in the following examples, which further defines the scope described herein. All references cited throughout the specification, and the references cited therein, are hereby expressly incorporated by reference in their entirety.

### Example 1

Isolation of Single-Domain Antibodies to the Receptor Binding Domain of SARS-CoV-2 from Immunized Llamas

One llama was immunized subcutaneously with SARS-CoV-2 virus antigens (recombinant protein of RBD (SEQ ID NO: 129) and Spike (SEQ ID NO: 130)) in the presence of Freund's adjuvant according to the scheme described in Table 6 below.

TABLE 6
Immunization protocol

| | | |
|---|---|---|
| | Day 0 | Bleed for test serum |
| Immu. 1 | Day 0 | 1 mg RBD, CFA |
| Immu. 2 | Day 14 | 0.5 mg RBD, IFA |
| | Day 24 | Bleed for test serum |
| Immu. 3 | Day 28 | 0.5 mg RBD, IFA |
| | Day 38 | Bleed for test serum |
| Immu. 4 | Day 42 | 0.5 mg RBD, IFA |
| | Day 52 | Bleed for test serum |
| Immu. 5 | Day 56 | 0.5 mg Spike, IFA |
| | Day 66 | Bleed for test serum |
| Immu. 6 | Day 70 | 0.5 mg Spike, IFA |
| | Day 80 | 500 ml blood |

The RBD polypeptide used for immunization includes the native Spike protein signal sequence at the N-terminus and is His-tagged at the C-terminus:

(SEQ ID NO: 129)
MFVFLVLLPLVSSQRVQPTESIVRFPNITNLCPFGEVFNATRFASVYAWN
RKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVG
YQPYRVVVLSFELLHAPATVCGPKKSTNLVKNKCVNFHHHHHH

The Spike polypeptide used for immunization includes its native signal sequence at the N-terminus, a stabilizing mutation in which 4 amino acids (RRAR at position 682 to 685 in the native Spike protein) are modified to a single alanine (A), and amino acids K986 and V987 are changed to PP (positions 983 and 984 in the below sequence), and is His-tagged at the C-terminus:

(SEQ ID NO: 130)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPONGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPASVASQSIIAYTMSLGAEN
SVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVN
QNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQYIKWPSGRLVPRGSPGSGYIPEAPRDGQAYVRKDGEWVLLSTFLG
HHHHHH

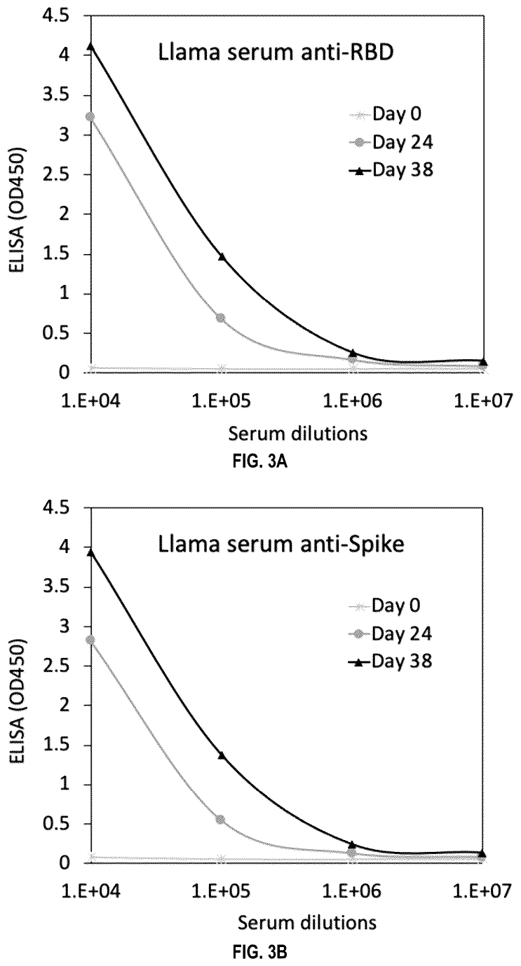

Test serum was collected on day 0, 24, 38, 52, 66 and 80 as indicated in Table 6. Immune response of the llama was analyzed for day 0, 24 and 38 serum samples by ELISA assay. To this end, recombinant RBD or Spike protein was captured in Maxisorp 96-well microtiter plates. After blocking, serial dilutions of serum samples were added, and bound llama IgG was detected by addition of goat anti-llama IgG-HRP (Invitrogen, A16060). Results are shown in FIGS. 3A & 3B. These data show that the immunized llama generated a good immune response against RBD, and the induced antibody can recognize Spike as well.

At day 80, 500 ml of whole blood was collected from the immunized llama and peripheral blood mononuclear cells (PBMC) were isolated using Ficoll-Paque plus (GE Healthcare) according to manufacturer's instructions. Total RNA extracted from PBMC was used as starting material for RT-PCR to amplify the VHH gene fragments by two steps of PCR reactions. These fragments were digested with Pstl and BstEll enzymes and cloned into pMES4 phagemid. Ligated vectors were electroporated into TG-1 bacteria (Lucigen) to construct VHH domain library. Library size was estimated to be 1.04E+10. VHH phage library was prepared by using VSCM13 helper phages and used for SARS-CoV-2 RBD antibody selection.

Single domain antibody fragments were selected using the VHH phage library described above and selections for RBD binders were performed with recombinant RBD of SARS-CoV-2 (SEQ ID NO: 129) as target protein. Two wells of Maxisorp 96-well plate were coated overnight in 4 degree with 50 ul of recombinant RBD protein (100 ug/ml diluted in PBS) and blocked with blocking buffer (5% non-fat milk in PBS). One well added with PBS was used as un-coated control. Phage library was blocked in room temperature for 1 hour in 5% non-fat milk and added to wells. After 2 hours incubation at room temperature wells were washed (15 times with 0.1% Tween-20 in PBS). Bound phages were eluted for 30 minutes with TrypLE™ Express Enzyme (ThermoFisher Scientific). 10 ul of phages eluted from RBD-coated or un-coated wells were serial diluted and used to infect TG-1 cells and plated. Selection efficiency was calculated based on the colonies grown from the coated and un-coated wells. The rest of phages eluted from RBD-coated wells were recovered by infecting TG-1 cells and incubated overnight in 37-degree bacteria shaker incubator.

The ratio of phages eluted from RBD-coated and un-coated wells reached to 280:1 after one round of selection. Recovered TG-1 cells were plated and individual colonies were picked to prepare periplasmic extracts comprising crude monoclonal single domain antibodies. Briefly, individual colonies were picked and grown in 96 deep-well plates (2×YT medium, 100 ug/ml Carbenicillin, 0.1% Glucose). Antibody expression was induced by adding IPTG (1 mM). Periplasmic extracts were prepared by resuspending bacteria pellet in 200 ul of PBS and rapid froze in liquid nitrogen. Frozen cells were thawed slowly in room temperature and centrifuged at 4200 rpm for 15 minutes. Single domain antibodies comprising supernatant was used for ELISA screening for RBD or Spike binders. To this end, recombinant RBD or Spike protein was captured in Maxisorp 96-well microtiter plates. After blocking, 100 ul of supernatant comprising antibodies was added, and bound llama IgG was detected by addition of goat anti-alpaca IgG (VHH domain)-HRP (Jackson ImmunoResearch, 128-035-232).

From this screening, 108 single-domain antibody candidates were identified and 50 candidates representing different CDR3 families were isolated for further testing.

### Example 2

Isolation of Single-Domain Antibodies to the Receptor Binding Domain of SARS-CoV-2 from Transgenic Mouse Model Expressing Camel, Alpaca and Dromedary Antibody Genes

We have generated a transgenic mouse model expressing camel, alpaca and dromedary antibody genes in place of endogenous mouse antibody gene loci. This transgenic mouse model was engineered by identifying 30 distinct immunoglobulin variable (VHH) domain genes from camel, dromedary and alpaca. The genes were synthesized and assembled into a 25 Kb minigene, which was then used to replace the entire VH domain of the mouse genome (2.5 Mb) in embryonic stem (ES) cells using CRISPR-Cas9 technology. In addition, the CH1 exon of IgM and IgG1 constant domains was deleted to abolish the pairing of light chain with heavy chain. Modified ES cells were used to generate chimera mice and F1 progeny carrying the VHH minigene knock-in were backcrossed with C57BL/6 mice. Homozygous mice carrying the VHH minigene knock-in were obtained by breeding heterozygous mice and used for immunizations.

Two groups of the homozygous transgenic mice described above (5 mice in group 1, 6 mice in group 2) were immunized with SARS-CoV-2 virus antigens (recombinant protein of RBD (SEQ ID NO: 129) and Spike (SEQ ID NO: 130)) in the presence of Freund's adjuvant according to the scheme described in Tables 7 and 8 below.

TABLE 7
Immunization protocol

| | | |
|---|---|---|
| Group 1 | Day 0 | Bleed for test serum |
| Immu. 1 | Day 0 | 50 ug RBD, CFA, i.p. |
| Immu. 2 | Day 14 | 25 ug RBD, IFA, i.p. |
| | Day 21 | Bleed for test serum |
| Immu. 3 | Day 28 | 25 ug RBD, IFA, i.p. |
| | Day 35 | Bleed for test serum |
| Immu. 4 | Day 42 | 25 ug RBD, IFA, i.p. |
| | Day 49 | Bleed for test serum |
| Immu. 5 | Day 56 | 25 ug Spike in PBS, i.p. |
| Immu. 6 | Day 59 | 25 ug Spike in PBS, i.v. |
| | Day 62 | Harvest Experiment |

TABLE 8
Immunization protocol

| | | |
|---|---|---|
| Group 2 | Day 0 | Bleed for test serum |
| Immu. 1 | Day 0 | 50 ug Spike, CFA, i.p. |
| Immu. 2 | Day 14 | 25 ug Spike, IFA, i.p. |
| | Day 21 | Bleed for test serum |
| Immu. 3 | Day 28 | 25 ug Spike, IFA, i.p. |
| | Day 35 | Bleed for test serum |
| Immu. 4 | Day 42 | 25 ug Spike, IFA, i.p. |
| | Day 49 | Bleed for test serum |
| Immu. 5 | Day 56 | 25 ug Spike in PBS, i.p. |
| Immu. 6 | Day 59 | 25 ug Spike in PBS, i.v. |
| | Day 62 | Harvest Experiment |

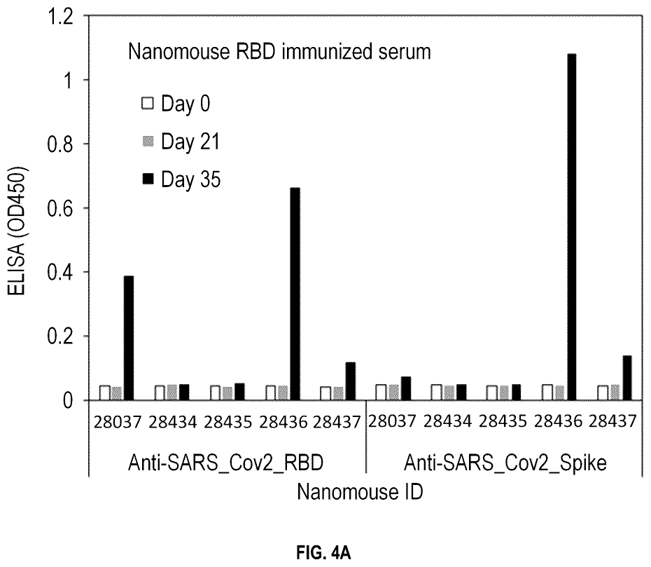

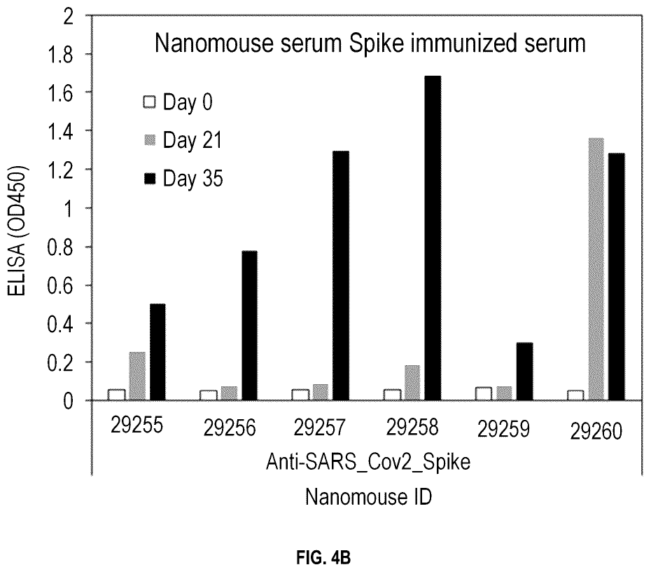

Test serum was collected on day 0, 21, 35, 49, 62 as indicated in Tables 7 and 8. Immune response of the immunized transgenic mice was analyzed for day 0, 21 and 35 serum samples by ELISA assay. To this end, recombinant RBD or Spike protein was captured in Maxisorp 96-well microtiter plates. After blocking, serial dilutions of serum samples were added, and bound mouse IgG was detected by addition of goat anti-mouse IgG-HRP. Results are shown in FIGS. 4A & 4B for Groups 1 and 2, respectively. These data show that immunized transgenic mice generated different level of immune responses and the best ones (#28436, #29258, #29260) were picked for single antibody library construction.

At day 62, splenic cells were collected from the immunized transgenic mice and total RNA extracted for RT-PCR to amplify the VHH-D-JH gene fragments. These fragments were digested with Sfil enzyme and cloned into pMES4 phagemid. Ligated vectors were electroporated into TG-1 bacteria (Lucigen) to construct single domain antibody library. Libraries size were estimated to be 9.68E+08, 1.35E+09 and 3.5E+09 for transgenic mice #28436, #29258 and #29260, respectively. VHH phage libraries were prepared by using VSCM13 helper phages and used for SARS-CoV-2 RBD and Spike antibody selection, the same way as llama VHH library screening.

Library from transgenic mouse #28436 was selected with RBD protein as target, and the ratio of phages eluted from RBD-coated and un-coated wells reached to 900:1 after one round of selection. Libraries from transgenic mice #29258 and #29260 were selected with Spike protein as target, and the ratio of phages eluted from Spike-coated and un-coated wells reached to 1600:1 and 700:1 after two rounds of selection.

Recovered TG-1 cells were plated and individual colonies were picked to prepare periplasmic extracts comprising crude monoclonal single domain antibodies, the same way as described in llama library screening.

From these screenings, in total 158 unique single-domain antibody candidates were identified and 63 candidates representing different CDR3 families were isolated for further testing.

### Example 3

Neutralization of SARS-CoV-2 Pseudotyped Viruses with Isolated Anti-SARS-CoV-2 Single-Domain Antibodies

Pseudotyped SARS-CoV-2 viruses were produced as described by Davide F. Robbiani et al (“Convergent antibody responses to SARS-CoV-2 in convalescent individuals.” Nature. 2020 August; 584(7821):437-442. doi: 10.1038/s41586-020-2456-9. Epub 2020 Jun. 18. PMID: 32555388). A plasmid expressing a C-terminally truncated SARS-CoV-2 S protein (pSARS-CoV2-Strunc) was generated by insertion of a human codon-optimized cDNA encoding SARS-CoV-2 S lacking the C-terminal 19 codons (Geneart) into pCR3.1. The S open-reading frame was taken from 'Wuhan seafood market pneumonia virus isolate Wuhan-Hu-1′ (GenBank: NC_045512). An env-inactivated HIV-1 reporter construct (pNL4-3ΔEnv-nanoluc) was generated from pNL4-332 by introducing a 940-bp deletion 3′ in the vpu stop codon, resulting in a frameshift in env. The human codon-optimized nanoluc Luciferase reporter gene (Nluc, Promega) was inserted in place of nucleotides 1-100 of the nef gene. To generate pseudotyped viral stocks, 293T cells were transfected with pNL4-3ΔEnv-nanoluc and pSARS-CoV2-Strunc or pSARS-CoV-S using polyethylenimine. Co-transfection of pNL4-3ΔEnv-nanoluc and S-expression plasmids leads to production of HIV-1-based virions that carried either the SARS-CoV-2 or SARS-CoV S protein on the surface. After transfection for 8 h, cells were washed twice with PBS and fresh medium was added. Supernatants comprising virions were collected 48 h after transfection, filtered and stored at −80° C.

Surrogate Virus Neutralization Test (sVNT) was performed according to the manufacturer's instructions (GenScript, L00847).

Pseudovirus neutralization assay was as described by Davide F. Robbiani et al (“Convergent antibody responses to SARS-CoV-2 in convalescent individuals.” Nature. 2020 August; 584(7821):437-442. doi: 10.1038/s41586-020-2456-9. Epub 2020 Jun. 18. PMID: 32555388). Fourfold serially diluted single domain antibodies were incubated with the SARS-CoV-2 pseudotyped virus for 1 h at 37° C. The mixture was subsequently incubated with 293TACE2 cells for 48 h after which cells were washed twice with PBS and lysed with Luciferase Cell Culture Lysis 5× reagent (Promega). Nanoluc Luciferase activity in lysates was measured using the Nano-Glo Luciferase Assay System (Promega) with Modulus II Microplate Reader User interface (TURNER BioSystems). The obtained relative luminescence units were normalized to those derived from cells infected with SARS-CoV-2 pseudotyped virus in the absence of antibodies. The half-maximal inhibitory concentration for single domain antibodies (IC50) was determined using four-parameter nonlinear regression (GraphPad Prism).

TABLE A
Neutralization of pseudotyped SARS-CoV-2 virus by single-
domain antibodies generated from llamas and transgenic
mice expressing camelid immunoglobulin genes.

| | IC50 | IC90 | IC50 | IC90 |
|---|---|---|---|---|
| ID | [ng/ml] | [ng/ml] | [nM] | [nM] |
| Nb1 | 596.4 | 3568.5 | 44.2 | 264.1 |
| Nb2 | nd | nd | nd | nd |
| Nb4 | 553.8 | 4485.3 | 41.0 | 331.9 |
| Nb6 | 141.2 | 354.1 | 10.5 | 26.3 |
| Nb7 | 43 | 450.9 | 3.2 | 33.4 |
| Nb11 | nd | nd | nd | nd |
| Nb12 | nd | nd | nd | nd |
| Nb13 | 354.4 | 1279.4 | 26.3 | 94.7 |
| Nb15 | 16.6 | 38.0 | 1.2 | 2.8 |
| Nb17 | 17.6 | 73.6 | 1.3 | 5.4 |
| Nb19 | 14.6 | 40.5 | 1.1 | 3.0 |
| Nb21 | 1897.6 | 3229.1 | 140.6 | 239.2 |
| Nb22 | nd | nd | nd | nd |
| mNb30 | 138.8 | 1078.9 | 10.3 | 80.1 |
| mNb34 | 149.2 | 1031.9 | 11.1 | 76.6 |
| mNb35 | 209.0 | 692.4 | 15.5 | 51.3 |
| Nb40 | nd | nd | nd | nd |
| Nb43 | 281.4 | 5327.2 | 20.9 | 394.8 |
| Nb44 | 848.6 | >10000 | 62.9 | >500 |
| Nb45 | nd | nd | nd | nd |
| Nb47 | 731.2 | >10000 | 54.2 | >500 |

nd = not detectable.

TABLE B
Surrogate virus neutralization test (sVNT) results
from selected llama-derived SARS-CoV-2 RBD antibodies.
Llama RBD derived antibodies

| ID | Inhibition Rate (%) |
|---|---|
| Nb1 | −3.7 |
| Nb2 | 4.2 |
| Nb4 | 19.6 |
| Nb6 | 22.4 |
| Nb7 | 73.9 |
| Nb11 | 12.2 |
| Nb12 | 9.8 |
| Nb13 | 13.6 |
| Nb15 | 93.6 |
| Nb17 | 87.0 |
| Nb18 | 6.0 |
| Nb19 | 92.6 |
| Nb21 | 14.0 |
| Nb22 | 10.3 |
| Nb40 | −1.9 |
| Nb43 | 23.1 |
| Nb44 | 10.9 |
| Nb45 | 12.8 |
| Nb47 | 22.0 |
| Nb48 | 1.6 |
| Nb49 | 0.0 |
| Nb50 | 36.5 |
| Nb51 | 7.6 |
| Nb52 | 14.7 |
| Nb53 | 28.3 |
| Nb54 | 14.8 |
| Nb55 | 22.3 |
| Nb56 | 84.9 |
| Nb57 | 0.4 |
| Nb58 | 10.3 |
| Nb59 | 14.1 |
| Nb60 | 78.8 |
| Nb61 | −0.6 |
| Nb62 | −5.6 |
| Nb63 | −5.2 |

TABLE C
Surrogate virus neutralization test (sVNT) results from
selected transgenic mouse-derived SARS-CoV-2 RBD antibodies.
Mouse RBD derived antibodies

| ID | Inhibition Rate (%) |
|---|---|
| mNb25 | 15.1 |
| mNb26 | 5.6 |
| mNb27 | 3.8 |
| mNb28 | 21.2 |
| mNb29 | 7.5 |
| mNb30 | 40.6 |
| mNb31 | 18.1 |
| mNb32 | 16.7 |
| mNb33 | −3.4 |
| mNb34 | 31.1 |
| mNb35 | 25.4 |
| mNb36 | 2.6 |
| mNb37 | 5.5 |

TABLE D
Surrogate virus neutralization test (sVNT) results from
selected transgenic mouse-derived SARS-CoV-2 Spike antibodies.
Mouse Spike derived antibodies

| ID | Inhibition Rate (%) |
|---|---|
| msNb1 | 1.9 |
| msNb2 | 5.3 |
| msNb3 | 11.2 |
| msNb4 | 13.3 |
| msNb5 | 13.3 |
| msNb6 | 10.9 |
| msNb7 | 4.3 |
| msNb8 | 5.5 |
| msNb9 | 12.4 |
| msNb10 | 11.6 |
| msNb11 | 13.3 |
| msNb12 | 34.6 |
| msNb13 | 18.1 |
| msNb14 | 11.8 |
| msNb15 | 10.6 |
| msNb16 | 3.9 |
| msNb17 | 4.9 |
| msNb18 | 15.1 |
| msNb19 | 46.0 |
| msNb20 | 15.3 |
| msNb21 | 12.2 |
| msNb22 | 11.3 |
| msNb23 | 6.9 |
| msNb24 | 8.8 |
| msNb25 | 5.5 |
| msNb26 | 11.5 |
| msNb27 | 11.3 |
| msNb28 | 12.5 |
| msNb29 | 20.7 |
| msNb30 | 18.4 |
| msNb31 | 20.3 |
| msNb32 | 2.5 |

Data from the above Tables A-D demonstrate that several single-domain antibodies generated against the RBD/Spike of SARS-CoV-2 were able to neutralize SARS-CoV-2. In particular, the most-effective single-domain antibodies derived from immunized llamas are Nb15, Nb17, Nb19, and Nb56. The most-effective single-domain antibodies derived from immunized transgenic mice capable of producing camelid antibodies are msNb12 and mNb30.

### Example 4

Neutralization of SARS-CoV-2 Pseudotyped Viruses with Isolated Anti-SARS-CoV-2 Single-Domain Antibody Fc Fusions

Monomeric or trimeric single domain antibodies were fused to the Fc region of human IgG1 molecule with additional 6×His tag on the C terminus. The Fc region contains a hinge region followed by CH2 and CH3 domain. In some cases, llama IgG2a hinge region was used in place of human IgG1 hinge. The Fc-fusion constructs were expressed in Expi293 cells and antibodies secreted into medium as dimeric Fc molecules, which were purified using cOmplete His-tag Purification Resin (Roche 05893801001) or POROS MabCapture A Select resin (Thermoscientific, A26457).

Table E below provides a full set of pseudovirus neutralization assay results for 6 selected single domain antibodies and their monomer/trimer Fc fusion version.

TABLE E
SARS-CoV-2 pseudovirus neutralization results

| | IC50 | IC90 | IC50 | IC90 |
|---|---|---|---|---|
| ID | [ng/ml] | [ng/ml] | [nM] | [nM] |
| Nb15 | 6.22 | 16.06 | 434.60 | 1122.24 |
| Nb17 | 7.98 | 37.26 | 577.91 | 2696.73 |
| Nb19 | 4.63 | 14.69 | 335.90 | 1063.56 |
| Nb56 | 13.71 | 27.21 | 931.94 | 1849.48 |
| msNb12 | 168.39 | 282.86 | 11716.29 | 19701.18 |
| mNb30 | 99.64 | 270.77 | 6924.85 | 18847.78 |
| Nb15-IgG1 | 14.25 | 32.79 | 357.03 | 821.21 |
| Nb17-IgG1 | 3.05 | 22.80 | 78.93 | 589.92 |
| Nb19-IgG1 | 2.54 | 10.51 | 65.79 | 271.87 |
| Nb56-IgG1 | 4.57 | 25.51 | 113.22 | 630.88 |
| msNb12-IgG2a | 29.95 | 65.80 | 700.84 | 1539.57 |
| mNb30-IgG2a | 81.68 | 299.91 | 1913.67 | 7038.87 |
| Nb15-tri-IgG1 | 10.02 | 26.91 | 142.02 | 381.37 |
| Nb17-tri-IgG1 | 1.24 | 4.70 | 17.96 | 68.21 |
| Nb19-tri-IgG1 | 0.65 | 3.96 | 9.38 | 57.62 |
| Nb56-tri-IgG1 | 3.91 | 18.65 | 54.47 | 259.50 |
| msNb12-tri-IgG2a | 4.71 | 74.04 | 64.85 | 1019.46 |
| mNb30-tri-IgG2a | 228.83 | 962.12 | 3153.11 | 13246.34 |

### Example 5

### Affinity of Anti-SARS-CoV-2 Single-Domain Antibodies for SARS-CoV-2 Polypeptides

The affinity of selected llama-derived and transgenic mouse-derived anti-SARS-CoV-2 antibodies and Fc fusion constructs thereof as described in Examples 1-4 for SARS-CoV-2 RBD and/or SARS-CoV-2 Spike polypeptides was determined.

Bio-Layer Interferometry (BLI) assay was performed to determine the affinity of single-domain antibodies to RBD. Briefly, biotinylated-RBD was immobilized onto streptavidin coated biosensors and then diluted single-domain antibodies were allowed to associate for 30 seconds followed by dissociation for 2-3 minutes. Curve fitting was applied using global fitting of the sensor data and a steady state analysis calculated to determine the association and dissociation constants.

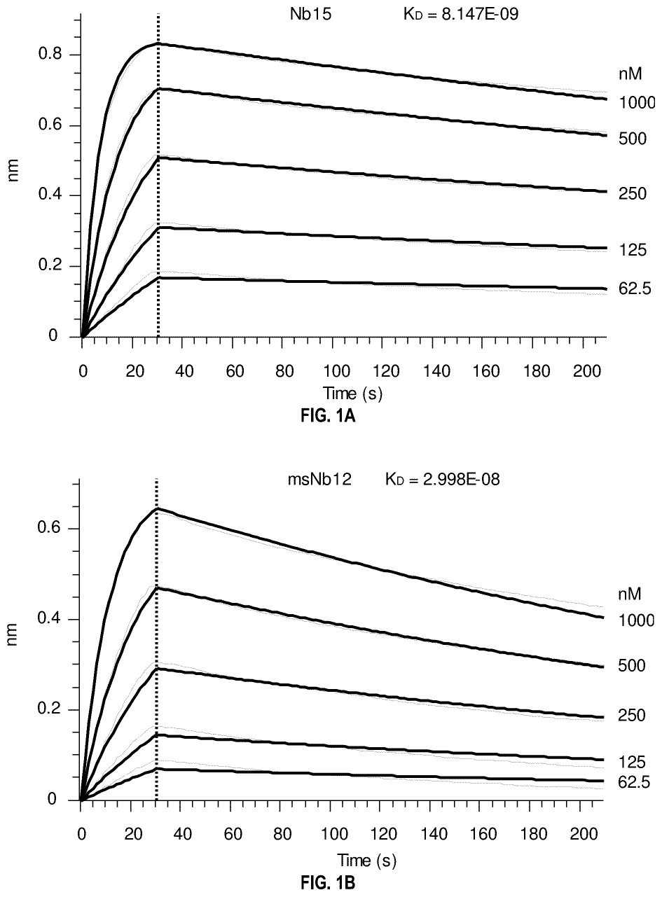

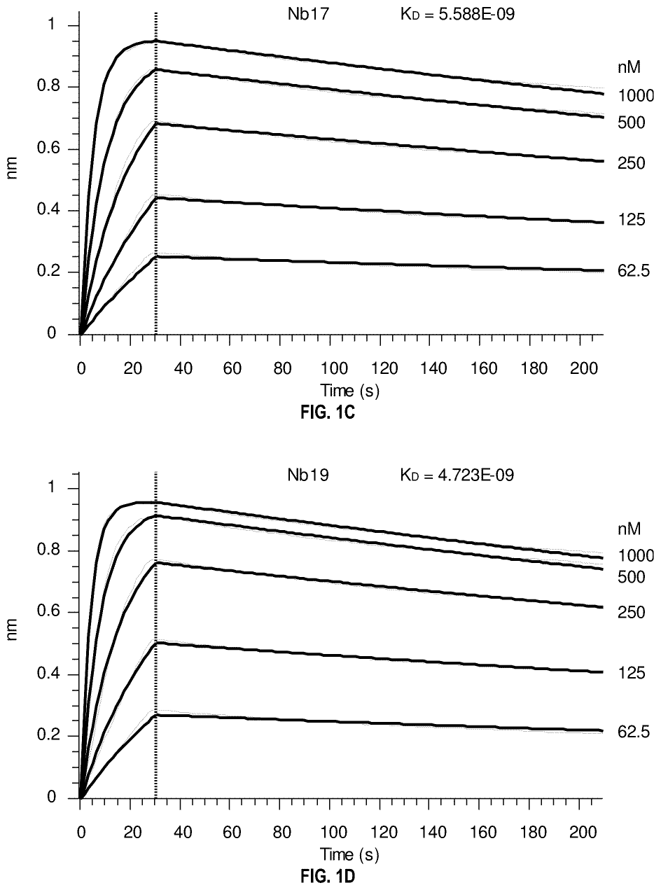

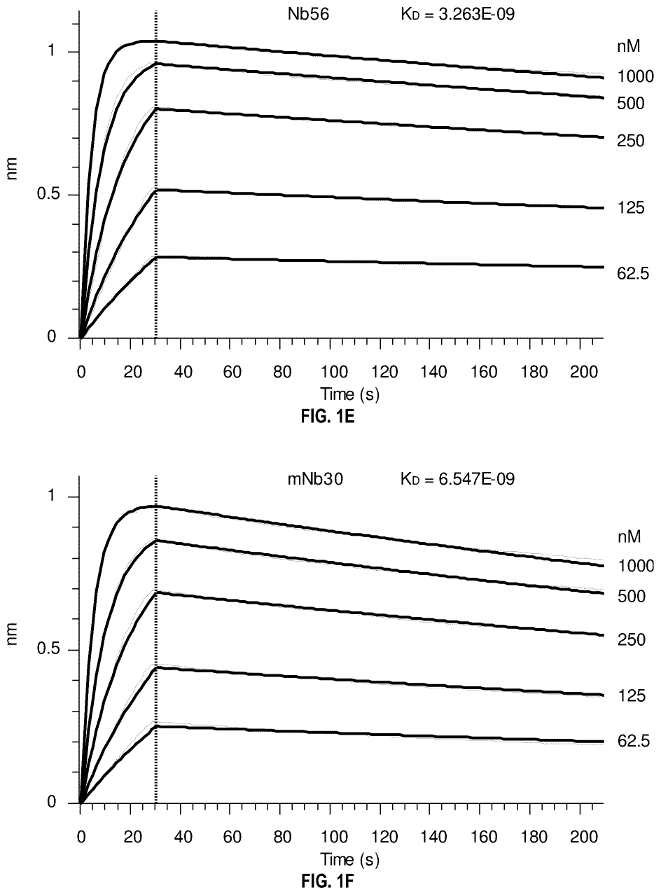

Results for six exemplary single-domain anti-SARS-CoV-2 antibodies (Nb15, msNb12, Nb17, Nb19, Nb56, mNb30) are provided in FIGS. 1A-1F.

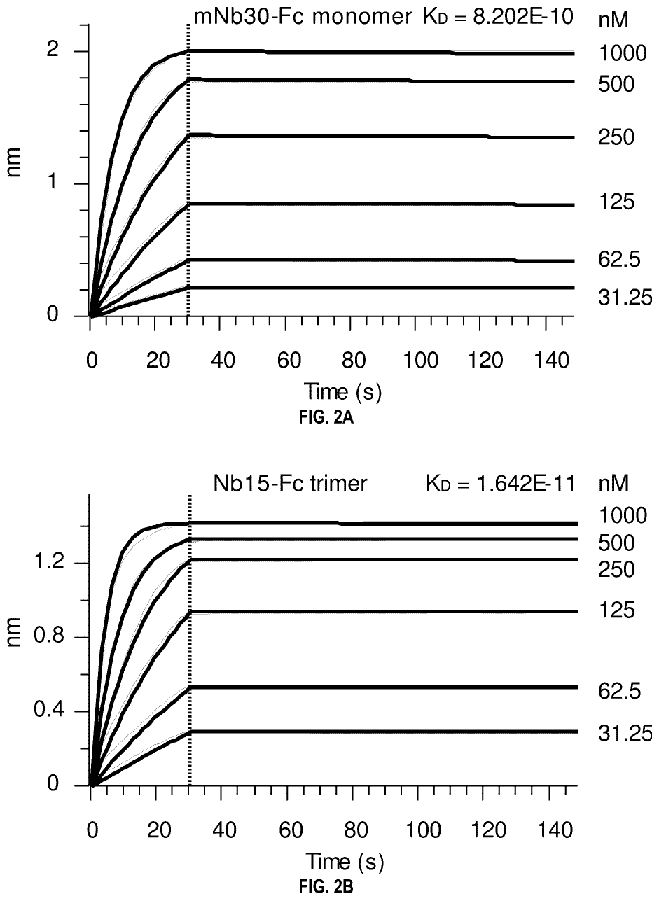

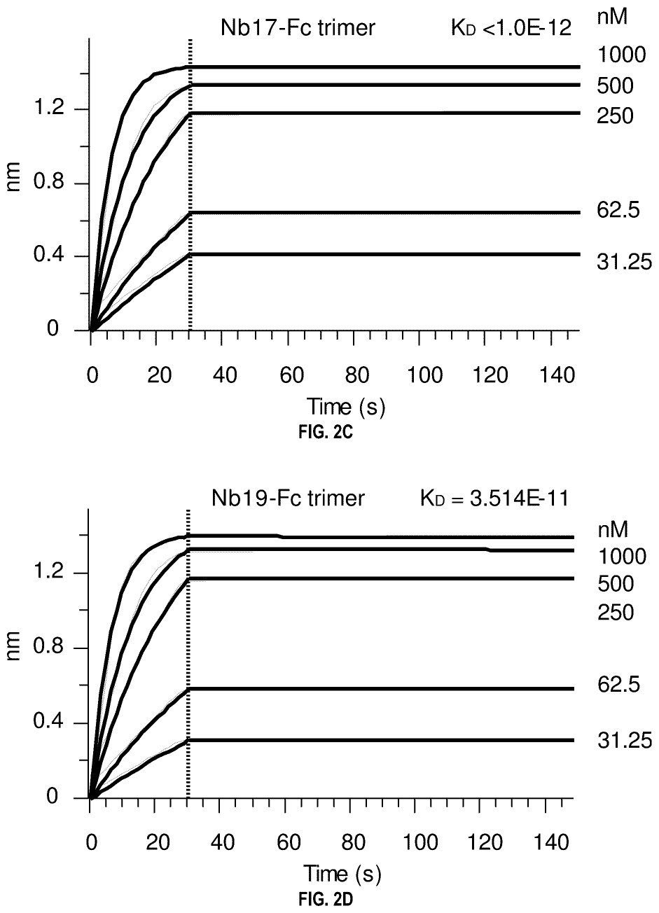

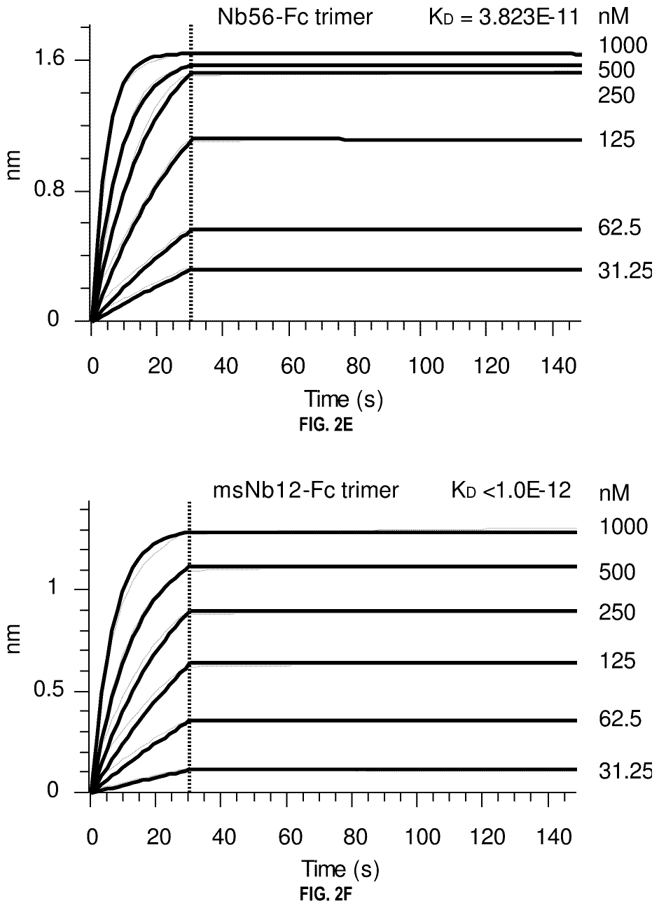

Results for Fc-fusions of six exemplary single-domain anti-SARS-CoV-2 antibodies (mNb30-IgG2a, Nb15-tri-IgG1, Nb17-tri-IgG1, Nb19-tri-IgG1, Nb56-tri-IgG1, msNb12-tri-IgG2a) are provided in FIGS. 2A-2F.

### Example 6

Neutralization of Mutant SARS-CoV-2 Pseudotyped Viruses with Isolated Anti-SARS-CoV-2 Single-Domain Antibodies

Select llama-derived and transgenic mouse-derived anti-SARS-CoV-2 antibodies and Fc fusion constructs thereof as described in Examples 1-4 were tested for their ability to neutralize SARS-CoV-2 variants using a pseudovirus neutralization assay as described above. All mutations were in the Spike protein. Results are shown below in Table F.

TABLE F
SARS-CoV-2 variant pseudovirus neutralization results

| | IC50 [pM] neutralizing SARS-CoV-2 mutations | | | | | |
|---|---|---|---|---|---|---|
| | WT | R683G | K417N | E484K | N501Y | KEN* |
| Nb15 | 320 | 73 | 237 | 1217 | 50000 | 50000 |
| Nb17 | 668 | 403 | 508 | 50000 | 5284 | 50000 |
| Nb19 | 309 | 122 | 394 | 50000 | 327 | 50000 |
| Nb56 | 533 | 237 | 436 | 15902 | 996 | 50000 |
| msNb12 | 7145 | 6910 | 6575 | 10048 | 5912 | 9658 |
| mNb30 | 3705 | 3837 | 2607 | 7353 | 3677 | 5240 |
| Nb15-tri-IgG1 | 73 | 13 | 27 | 41 | 21 | 30 |
| Nb17-tri-IgG1 | 15 | 4 | 8 | 50000 | 10 | 47350 |
| Nb19-tri-IgG1 | 12 | 3 | 6 | 95 | 10 | 115 |
| Nb56-tri-IgG1 | 43 | 8 | 21 | 37 | 28 | 14 |
| msNb12-tri- | 91 | 178 | 68 | 149 | 82 | 90 |
| IgG2a | | | | | | |
| mNb30-IgG2a | 614 | 464 | 389 | 1151 | 552 | 858 |

*KEN = K417N + E484K + N501Y

This data demonstrates that the single-domain antibodies disclosed herein are able to neutralize SARS-CoV-2 variants as well as wild-type SARS-CoV-2.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1-70. (canceled)

71. An isolated single-domain antibody, or an antigen binding fragment thereof, comprising a complementarity determining region 3 (CDR3), wherein:
an amino acid sequence of the CDR3 is selected from the group consisting of:
an amino acid sequence of any one of SEQ ID NOS: 77 to 91;
an amino acid sequence having at least 80% identity to at least one of the amino acid sequences of SEQ ID NOS: 77 to 91;
an amino acid sequence having 3, 2, or 1 amino acid differences with at least one of the amino acid sequences of SEQ ID NOS: 77 to 91; and
the antigen is a receptor binding domain of SARS-CoV-2 (SARS-CoV-2 RBD).

72. The single-domain antibody, or antigen binding fragment thereof, of claim 71, further comprising a complementarity determining region 1 (CDR1), or a complementarity determining region 2 (CDR2), or both, wherein:
an amino acid sequence of the CDR1 is selected from the group consisting of:
an amino acid sequence of any one of SEQ ID NOS: 27 to 40,
an amino acid sequence having at least 80% identity to at least one of the amino acid sequences of SEQ ID NOS: 27 to 40, and
an amino acid sequence having 3, 2, or 1 amino acid differences with at least one of the amino acid sequences of SEQ ID NOS: 27 to 40; and
an amino acid sequence of the CDR2 is selected from the group consisting of:
an amino acid sequence of any one of SEQ ID NOS: 49 to 62,
an amino acid sequence having at least 80% identity to at least one of the amino acid sequences of SEQ ID NOS: 49 to 62, and
an amino acid sequence having 3, 2, or 1 amino acid differences with at least one of the amino acid sequences of SEQ ID NOS: 49 to 62.

73. The single-domain antibody, or antigen binding fragment thereof, of claim 71, wherein the respective amino acid sequences of the respective CDRs of the single-domain antibody, or antigen binding fragment thereof each have at least 70% identity to a respective sequence of at least one amino acid sequence of SEQ ID NOS: 1 to 15.

74. The single-domain antibody, or antigen binding fragment thereof, of claim 71, further comprising a framework region 1 (FR1), framework region 2 (FR2), framework region 1 (FR3), or a framework region 1 (FR4), or any combination thereof, wherein:
an amino acid sequence of the FR1 is selected from the group consisting of:
an amino acid sequence of any one of SEQ ID NOS: 16 to 26,
an amino acid sequence having at least 80% identity with at least one of the amino acid sequences of SEQ ID NOS: 16 to 26,
an amino acid sequence having 5, 4, 3, 2, or 1 amino acid differences with at least one of the amino acid sequences of SEQ ID NOS: 16 to 26, and
an amino acid sequence of the FR2 is selected from the group consisting of:
an amino acid sequence of any one of SEQ ID NOS: 41 to 48,
an amino acid sequence having at least 80% identity with at least one of the amino acid sequences of SEQ ID NOS: 41 to 48,
an amino acid sequence having 5, 4, 3, 2, or 1 amino acid differences with at least one of the amino acid sequences of SEQ ID NOS: 41 to 48;
an amino acid sequence of the FR3 is selected from the group consisting of:
an amino acid sequence of any one of SEQ ID NOS: 63 to 76,
an amino acid sequence having at least 80% identity with at least one of the amino acid sequences of SEQ ID NOS: 63 to 76,
an amino acid sequence having 5, 4, 3, 2, or 1 amino acid differences with at least one of the amino acid sequences of SEQ ID NOS: 63 to 76; and
an amino acid sequence of the FR4 is selected from the group consisting of:
an amino acid sequence of any one of SEQ ID NOS: 92 to 96,
an amino acid sequence having at least 80% identity with at least one of the amino acid sequences of SEQ ID NOS: 92 to 96,
an amino acid sequence having 5, 4, 3, 2, or 1 amino acid differences with at least one of the amino acid sequences of SEQ ID NOS: 92 to 96.

75. The single-domain antibody, or antigen binding fragment thereof, of claim 71, having an amino acid sequence comprising an amino acid sequence selected from the group consisting of an amino acid sequence having at least 70% identity to an amino acid sequence selected from the group consisting of SEQ ID NOS: 1 to 15.

76. The single-domain antibody, or antigen binding fragment thereof, of claim 71, having an amino acid sequence comprising an amino acid sequence selected from the group consisting of SEQ ID NOS: 1 to 15.

77. The single-domain antibody, or antigen binding fragment thereof, of claim 71, encoded by a nucleic acid comprising a nucleotide sequence having at least 70% identity to a nucleic acid sequence selected from the group consisting of SEQ ID NOS: 97 to 111.

78. The single-domain antibody, or antigen binding fragment thereof, of claim 71, having an amino acid sequence comprising an amino acid sequence selected from the group consisting of SEQ ID NOS: 112 to 123.

79. The single-domain antibody, or antigen binding fragment thereof, of claim 71, wherein the SARS-CoV-2 RBD has an amino acid sequence comprising an amino acid sequence of SEQ ID NO: 124 or SEQ ID NO: 127.

80. The single-domain antibody, or antigen binding fragment thereof, of claim 71, wherein the single-domain antibody prevents the receptor binding domain from binding angiotensin converting enzyme 2 (ACE2).

81. A nucleic acid or nucleotide sequence encoding the single-domain antibody or antigen binding fragment thereof of claim 71.

82. A vector comprising the nucleic acid or nucleotide sequence of claim 81.

83. A host or host cell comprising the nucleic acid or nucleotide sequence of claim 81.

84. A pharmaceutical composition comprising the single-domain antibody or antigen binding fragment thereof of claim 71 and a pharmaceutical excipient, carrier, diluent, adjuvant, or a combination thereof.

85. A method for detecting a coronavirus polypeptide comprising contacting a sample with the single-domain antibody, or antigen binding fragment thereof, of claim 71.

86. The method of claim 85, wherein the single-domain antibody, or antigen binding fragment thereof, or polypeptide is attached to a solid phase support.

87. A method for treating and/or preventing a coronavirus infection comprising administering, to a patient in need thereof, an effective amount of the single-domain antibody, or antigen binding fragment thereof, of claim 71.

88. A method for treating a condition associated with a coronavirus infection comprising administering, to a patient in need thereof, an effective amount of the single-domain antibody, or antigen binding fragment thereof, of claim 71.

89. A kit comprising the single-domain antibody or antigen binding fragment of claim 71.

90. A device configured for inhalation delivery comprising the single-domain antibody or antigen binding fragment of claim 71.


SEQUENCE LISTING (130 sequences)

SEQ ID NO: 1 - protein, 124 aa, Artificial Sequence
  Single-domain antibody sequence (Nb15)
QVQLQESGGGLVQAGGSLRVSCAASGLPFSDYLMGWFRQAPGKEREYVAAISQNGGHTYADSVLGRFTISRDNAKNTVYLQMNMLTPGDTAVYSCAARRPGGGRWDAAHDYNYWGQGTQVTVSS

SEQ ID NO: 2 - protein, 119 aa, Artificial Sequence
  Single-domain antibody sequence (Nb17)
QVQLQESGGGLVQTGGSLRLSCAASGRTFGIYRMGWFRQAPGKEREFVAAITSSADTAQYRDSVKGRFAISRDNAKNTLYLQMNSLKPEDTAIYYCAARDPTTLEYGNWGQGTQVTVSS

SEQ ID NO: 3 - protein, 119 aa, Artificial Sequence
  Single-domain antibody sequence (Nb19)
QVQLQESGGGLVQAGGSLRLSCAASGSGFSIHAMGWYRQAPGKQREFVAVVGHKTNYADSVKGRFTISRDVGKNTVELQMNSLKVEDTAVYYCYCNTIVTMTGVPDAVWGQGTQVTVSS

SEQ ID NO: 4 - protein, 128 aa, Artificial Sequence
  Single-domain antibody sequence (Nb56)
QVQLQESGGGLVQAGDSLRLSCVASERTFRRYGMGWFRQAPGKEREFVAAVDRSHTKTGYADFVKGRFTISTNYENMVYLQMNSLKPEDTAVYYCAAPSYEKGSDPTSWNTDRGYDYWGQGTQVTVSS

SEQ ID NO: 5 - protein, 114 aa, Artificial Sequence
  Single-domain antibody sequence (Nb6)
QVQLQESGGGLVQAGGSLRLSCAASGNIANSNSMAWWRQTPGNQHERVAIISHGVTNYADSVKGRFTVSRDNAKNTLYLQMNNLKPEDTAAYYCYADLFGNTYWGQGTQVTVSS

SEQ ID NO: 6 - protein, 129 aa, Artificial Sequence
  Single-domain antibody sequence (Nb7)
QVQLQESGGGLVQAGDSLRLSCLSSERTFRRYGIAWFRQAPGKEREFVAAVDRSHSQTNYADFVQGRFTISTVYAKNMVYLQMNSLKPEDTAVYYCAAASYEKGSDYTSWNTDRGYDYWGQGTQVTVSS

SEQ ID NO: 7 - protein, 128 aa, Artificial Sequence
  Single-domain antibody sequence (Nb43)
QVQLQESGGGLVQAGDSLKLSCAASGRTFSDSAMGWFRQAPGEEREFVAVITWNGGTTYYADSVKGRFTISRDDAKNTVYLQMNSLKPEDTAVYYCAADTSRWDYSLTYHYTREYNYWGQGTQVTVSS

SEQ ID NO: 8 - protein, 130 aa, Artificial Sequence
  Single-domain antibody sequence (Nb47)
QVQLQESGGGLVQAGGSLRLSCAASGRTLSRYSMSWFRQAPGKEREFVAGIRWSGSNTYYADSMKQRFTISQDNVKNTVHLQMNSLKPEDTAVYYCAATSRSDHYLNAVAWTLPNEYDYWGQGTQVTVSS

SEQ ID NO: 9 - protein, 120 aa, Artificial Sequence
  Single-domain antibody sequence (Nb50)
QVQLQESGGGLVQAGGSLRLSCAASGRTFDGTYRMGWFRQGPGKEREFVAAIGFGVSTTSYADSVKGRFTISRNNAKNTVYLQMNSLKPEDTAVYYCAARPPPYTEYNYWGQGTQVTVSS

SEQ ID NO: 10 - protein, 124 aa, Artificial Sequence
  Single-domain antibody sequence (Nb53)
QVQLQESGGGVVQAGDSLRLSCAASGLTFSSYLMAWFRQAPGKEREFVARINWNGRVPYTVDSVKGRFIISRDNAKNTVWLQMNSLKPEDTAVYYCAADRNYGTGGAETVYEYWGQGTQVTVSS

SEQ ID NO: 11 - protein, 125 aa, Artificial Sequence
  Single-domain antibody sequence (Nb55)
QVQLQESGGGLVQAGGSLRLSCEASGRAFSDSSAMAWFRQAPGKEREFVAALNRVNVAYCRDSVSGRFTISRDNGKNTAYLEMNSVKPEDTAIYYCASDLILNDCSRNPARYAYWGQGTQVTVSS

SEQ ID NO: 12 - protein, 129 aa, Artificial Sequence
  Single-domain antibody sequence (Nb60)
QVQLQESGGGLVQAGDSLRLSCLASERTFRRYGMGWFRQAPGKEREFVAAIDRSHSNTDYADFVKGRFTISTVYAKNMVYLQMNSLKPEDTAVYYCAAATYEKGSDPTSWNTDRGYDVWGQGTQVTVSS

SEQ ID NO: 13 - protein, 121 aa, Artificial Sequence
  Single-domain antibody sequence (msNb12)
QVKLEESGGGSVQAGGSLRLICTAPGLTHNNCGLDWYRRAPGKEREFVSSISADGTTSYADSVKGRFTISKDKVEDTVYLQMNSLKPEDTAIYSCKTAFPYFGNSCVLDYWGQGTSVTVSS

SEQ ID NO: 14 - protein, 121 aa, Artificial Sequence
  Single-domain antibody sequence (mNb30)
QVQLVESGGGLVQAGGSLRLSCAASGLTFSKYAMGWFRQAPGKERKFVATISWSGDSAFYADSVKGRFTISRDNARNTVYLQMNSLKPEDTAVYYCAADRGMGYGDFMDYWGQGTSVTASS

SEQ ID NO: 15 - protein, 121 aa, Artificial Sequence
  Single-domain antibody sequence (mNb35)
QVQLVESGGGLVQAGGSLRLSCAASGRTFSKYAMGWFRQAPGKERKFVATISWSGDSAFYADSVKGRFTISRDNARNTVYLQMNSLKPEDTAVYYCTADRGMGYGDFMDYWGQGTSVTVSS

SEQ ID NO: 16 - protein, 26 aa, Artificial Sequence
  FR1 sequence
QVQLQESGGGLVQAGGSLRVSCAASG

SEQ ID NO: 17 - protein, 26 aa, Artificial Sequence
  FR1 sequence
QVQLQESGGGLVQTGGSLRLSCAASG

SEQ ID NO: 18 - protein, 26 aa, Artificial Sequence
  FR1 sequence
QVQLQESGGGLVQAGGSLRLSCAASG

SEQ ID NO: 19 - protein, 26 aa, Artificial Sequence
  FR1 sequence
QVQLQESGGGLVQAGDSLRLSCVASE

SEQ ID NO: 20 - protein, 26 aa, Artificial Sequence
  FR1 sequence
QVQLQESGGGLVQAGDSLRLSCLSSE

SEQ ID NO: 21 - protein, 26 aa, Artificial Sequence
  FR1 sequence
QVQLQESGGGLVQAGDSLKLSCAASG

SEQ ID NO: 22 - protein, 26 aa, Artificial Sequence
  FR1 sequence
QVQLQESGGGVVQAGDSLRLSCAASG

SEQ ID NO: 23 - protein, 26 aa, Artificial Sequence
  FR1 sequence
QVQLQESGGGLVQAGGSLRLSCEASG

SEQ ID NO: 24 - protein, 26 aa, Artificial Sequence
  FR1 sequence
QVQLQESGGGLVQAGDSLRLSCLASE

SEQ ID NO: 25 - protein, 26 aa, Artificial Sequence
  FR1 sequence
QVKLEESGGGSVQAGGSLRLICTAPG

SEQ ID NO: 26 - protein, 26 aa, Artificial Sequence
  FR1 sequence
QVQLVESGGGLVQAGGSLRLSCAASG

SEQ ID NO: 27 - protein, 9 aa, Artificial Sequence
  CDR1 sequence
LPFSDYLMG

SEQ ID NO: 28 - protein, 9 aa, Artificial Sequence
  CDR1 sequence
RTFGIYRMG

SEQ ID NO: 29 - protein, 9 aa, Artificial Sequence
  CDR1 sequence
SGFSIHAMG

SEQ ID NO: 30 - protein, 9 aa, Artificial Sequence
  CDR1 sequence
RTFRRYGMG

SEQ ID NO: 31 - protein, 9 aa, Artificial Sequence
  CDR1 sequence
NIANSNSMA

SEQ ID NO: 32 - protein, 9 aa, Artificial Sequence
  CDR1 sequence
RTFRRYGIA

SEQ ID NO: 33 - protein, 9 aa, Artificial Sequence
  CDR1 sequence
RTFSDSAMG

SEQ ID NO: 34 - protein, 9 aa, Artificial Sequence
  CDR1 sequence
RTLSRYSMS

SEQ ID NO: 35 - protein, 10 aa, Artificial Sequence
  CDR1 sequence
RTFDGTYRMG

SEQ ID NO: 36 - protein, 9 aa, Artificial Sequence
  CDR1 sequence
LTFSSYLMA

SEQ ID NO: 37 - protein, 10 aa, Artificial Sequence
  CDR1 sequence
RAFSDSSAMA

SEQ ID NO: 38 - protein, 9 aa, Artificial Sequence
  CDR1 sequence
LTHNNCGLD

SEQ ID NO: 39 - protein, 9 aa, Artificial Sequence
  CDR1 sequence
LTFSKYAMG

SEQ ID NO: 40 - protein, 9 aa, Artificial Sequence
  CDR1 sequence
RTFSKYAMG

SEQ ID NO: 41 - protein, 14 aa, Artificial Sequence
  FR2 sequence
WFRQAPGKEREYVA

SEQ ID NO: 42 - protein, 14 aa, Artificial Sequence
  FR2 sequence
WFRQAPGKEREFVA

SEQ ID NO: 43 - protein, 14 aa, Artificial Sequence
  FR2 sequence
WYRQAPGKQREFVA

SEQ ID NO: 44 - protein, 14 aa, Artificial Sequence
  FR2 sequence
WWRQTPGNQHERVA

SEQ ID NO: 45 - protein, 14 aa, Artificial Sequence
  FR2 sequence
WFRQAPGEEREFVA

SEQ ID NO: 46 - protein, 14 aa, Artificial Sequence
  FR2 sequence
WFRQGPGKEREFVA

SEQ ID NO: 47 - protein, 14 aa, Artificial Sequence
  FR2 sequence
WYRRAPGKEREFVS

SEQ ID NO: 48 - protein, 14 aa, Artificial Sequence
  FR2 sequence
WFRQAPGKERKFVA

SEQ ID NO: 49 - protein, 9 aa, Artificial Sequence
  CDR2 sequence
AISQNGGHT

SEQ ID NO: 50 - protein, 10 aa, Artificial Sequence
  CDR2 sequence
AITSSADTAQ

SEQ ID NO: 51 - protein, 7 aa, Artificial Sequence
  CDR2 sequence
VVGHKTN

SEQ ID NO: 52 - protein, 10 aa, Artificial Sequence
  CDR2 sequence
AVDRSHTKTG

SEQ ID NO: 53 - protein, 8 aa, Artificial Sequence
  CDR2 sequence
IISHGVTN

SEQ ID NO: 54 - protein, 10 aa, Artificial Sequence
  CDR2 sequence
AVDRSHSQTN

SEQ ID NO: 55 - protein, 10 aa, Artificial Sequence
  CDR2 sequence
VITWNGGTTY

SEQ ID NO: 56 - protein, 10 aa, Artificial Sequence
  CDR2 sequence
GIRWSGSNTY

SEQ ID NO: 57 - protein, 10 aa, Artificial Sequence
  FR2 sequence
AIGFGVSTTS

SEQ ID NO: 58 - protein, 10 aa, Artificial Sequence
  CDR2 sequence
RINWNGRVPY

SEQ ID NO: 59 - protein, 9 aa, Artificial Sequence
  CDR2 sequence
ALNRVNVAY

SEQ ID NO: 60 - protein, 10 aa, Artificial Sequence
  CDR2 sequence
AIDRSHSNTD

SEQ ID NO: 61 - protein, 9 aa, Artificial Sequence
  CDR2 sequence
SISADGTTS

SEQ ID NO: 62 - protein, 10 aa, Artificial Sequence
  CDR2 sequence
TISWSGDSAF

SEQ ID NO: 63 - protein, 37 aa, Artificial Sequence
  FR3 sequence
YADSVLGRFTISRDNAKNTVYLQMNMLTPGDTAVYSC

SEQ ID NO: 64 - protein, 37 aa, Artificial Sequence
  FR3 sequence
YRDSVKGRFAISRDNAKNTLYLQMNSLKPEDTAIYYC

SEQ ID NO: 65 - protein, 37 aa, Artificial Sequence
  FR3 sequence
YADSVKGRFTISRDVGKNTVELQMNSLKVEDTAVYYC

SEQ ID NO: 66 - protein, 36 aa, Artificial Sequence
  FR3 sequence
YADFVKGRFTISTNYENMVYLQMNSLKPEDTAVYYC

SEQ ID NO: 67 - protein, 37 aa, Artificial Sequence
  FR3 sequence
YADSVKGRFTVSRDNAKNTLYLQMNNLKPEDTAAYYC

SEQ ID NO: 68 - protein, 37 aa, Artificial Sequence
  FR3 sequence
YADFVQGRFTISTVYAKNMVYLQMNSLKPEDTAVYYC

SEQ ID NO: 69 - protein, 37 aa, Artificial Sequence
  FR3 sequence
YADSVKGRFTISRDDAKNTVYLQMNSLKPEDTAVYYC

SEQ ID NO: 70 - protein, 37 aa, Artificial Sequence
  FR3 sequence
YADSMKQRFTISQDNVKNTVHLQMNSLKPEDTAVYYC

SEQ ID NO: 71 - protein, 37 aa, Artificial Sequence
  FR3 sequence
YADSVKGRFTISRNNAKNTVYLQMNSLKPEDTAVYYC

SEQ ID NO: 72 - protein, 37 aa, Artificial Sequence
  FR3 sequence
TVDSVKGRFIISRDNAKNTVWLQMNSLKPEDTAVYYC

SEQ ID NO: 73 - protein, 37 aa, Artificial Sequence
  FR3 sequence
CRDSVSGRFTISRDNGKNTAYLEMNSVKPEDTAIYYC

SEQ ID NO: 74 - protein, 37 aa, Artificial Sequence
  FR3 sequence
YADFVKGRFTISTVYAKNMVYLQMNSLKPEDTAVYYC

SEQ ID NO: 75 - protein, 37 aa, Artificial Sequence
  FR3 sequence
YADSVKGRFTISKDKVEDTVYLQMNSLKPEDTAIYSC

SEQ ID NO: 76 - protein, 37 aa, Artificial Sequence
  FR3 sequence
YADSVKGRFTISRDNARNTVYLQMNSLKPEDTAVYYC

SEQ ID NO: 77 - protein, 17 aa, Artificial Sequence
  CDR3 sequence
AARRPGGGRWDAAHDYN

SEQ ID NO: 78 - protein, 11 aa, Artificial Sequence
  CDR3 sequence
AARDPTTLEYG

SEQ ID NO: 79 - protein, 14 aa, Artificial Sequence
  CDR3 sequence
YCNTIVTMTGVPDA

SEQ ID NO: 80 - protein, 21 aa, Artificial Sequence
  CDR3 sequence
AAPSYEKGSDPTSWNTDRGYD

SEQ ID NO: 81 - protein, 8 aa, Artificial Sequence
  CDR3 sequence
YADLFGNT

SEQ ID NO: 82 - protein, 21 aa, Artificial Sequence
  CDR3 sequence
AAASYEKGSDYTSWNTDRGYD

SEQ ID NO: 83 - protein, 20 aa, Artificial Sequence
  CDR3 sequence
AADTSRWDYSLTYHYTREYN

SEQ ID NO: 84 - protein, 22 aa, Artificial Sequence
  CDR3 sequence
AATSRSDHYLNAVAWTLPNEYD

SEQ ID NO: 85 - protein, 11 aa, Artificial Sequence
  CDR3 sequence
AARPPPYTEYN

SEQ ID NO: 86 - protein, 16 aa, Artificial Sequence
  CDR3 sequence
AADRNYGTGGAETVYE

SEQ ID NO: 87 - protein, 17 aa, Artificial Sequence
  CDR3 sequence
ASDLILNDCSRNPARYA

SEQ ID NO: 88 - protein, 21 aa, Artificial Sequence
  CDR3 sequence
AAATYEKGSDPTSWNTDRGYD

SEQ ID NO: 89 - protein, 14 aa, Artificial Sequence
  CDR3 sequence
KTAFPYFGNSCVLD

SEQ ID NO: 90 - protein, 13 aa, Artificial Sequence
  CDR3 sequence
AADRGMGYGDFMD

SEQ ID NO: 91 - protein, 13 aa, Artificial Sequence
  CDR3 sequence
TADRGMGYGDFMD

SEQ ID NO: 92 - protein, 12 aa, Artificial Sequence
  FR4 sequence
YWGQGTQVTVSS

SEQ ID NO: 93 - protein, 12 aa, Artificial Sequence
  FR4 sequence
NWGQGTQVTVSS

SEQ ID NO: 94 - protein, 12 aa, Artificial Sequence
  FR4 sequence
VWGQGTQVTVSS

SEQ ID NO: 95 - protein, 12 aa, Artificial Sequence
  FR4 sequence
YWGQGTSVTVSS

SEQ ID NO: 96 - protein, 12 aa, Artificial Sequence
  FR4 sequence
YWGQGTSVTASS

SEQ ID NO: 97 - DNA, 372 bp, Artificial Sequence
  Single-domain antibody nt sequence (Nb15)
caggtgcagctgcaggagtctgggggagggttggtgcaggctgggggctctctgagagtctcctgtgcagcgtctggacttcccttcagtgactatttaatgggctggttccgccaggcgccagggaaggagcgtgagtatgtagccgctattagtcagaatggtggacacacttatgcagactccgtgctgggccgattcaccatctccagagacaacgccaagaatacggtgtatctgcaaatgaacatgttgacacctggggacacggccgtttatagttgtgctgcccgaaggcccggtgggggtaggtgggatgccgcccatgactataactactggggccaggggacccaggtcaccgtctcctca

SEQ ID NO: 98 - DNA, 357 bp, Artificial Sequence
  Single-domain antibody nt sequence (Nb17)
caggtgcagctgcaggagtctgggggaggattggtgcaaactgggggctctctgagactctcctgtgcagcctctggacgcaccttcggtatctatcgcatgggctggttccgccaggctccagggaaggagcgtgagtttgtagcagctatcacttcgagtgctgataccgcacagtatcgagactccgtgaagggccgattcgccatctccagagacaacgccaagaacacgctgtatctgcaaatgaacagcctgaaacctgaggacacggccatttattattgtgcagcacgggatcccactacattggagtatggcaactggggccaggggacccaggtcaccgtctcctca

SEQ ID NO: 99 - DNA, 357 bp, Artificial Sequence
  Single-domain antibody nt sequence (Nb19)
caggtgcagctgcaggagtctgggggaggcttggtgcaggctggggggtctctgagactgtcctgtgcagcctctggaagcggctttagtattcatgccatgggctggtaccgccaggctccagggaagcagcgcgagttcgtcgctgtcgttggccataagacgaactatgcagactccgttaagggccgattcaccatctccagagacgttggcaagaacacggtggagctgcaaatgaacagcctgaaagttgaggacacagccgtctattattgttactgcaatactatcgtgactatgacaggggttcctgatgccgtctggggccaggggacccaggtcaccgtctcctca

SEQ ID NO: 100 - DNA, 384 bp, Artificial Sequence
  Single-domain antibody nt sequence (Nb56)
caggtgcagctgcaggagtctgggggaggattggtgcaggctggggactctctgagactctcctgtgtagcctctgaacgcacattcaggcgctatggcatgggctggttccgccaggctccagggaaggagcgtgagtttgtagcagctgttgaccggagtcatactaagacaggctatgcagacttcgtgaagggccgattcaccatctccacgaactacgagaacatggtgtatctgcaaatgaacagcctgaaacctgaggacacggccgtttattactgtgctgcgccgtcgtacgagaaagggtcggaccctactagttggaacaccgacagagggtatgactactggggccaggggacccaggtcaccgtctcctca

SEQ ID NO: 101 - DNA, 342 bp, Artificial Sequence
  Single-domain antibody nt sequence (Nb6)
caggtgcagctgcaggagtctgggggaggcttggtgcaggctggggggtctctgagactctcctgtgcagcctctggaaacatcgcgaattccaattccatggcctggtggcgccagactccaggaaaccagcacgagcgggtcgccattattagtcatggtgtaacaaactatgcagattccgtgaagggccgattcacagtgtccagagacaacgccaagaatactttgtatctgcaaatgaacaacctgaaacctgaggacacagccgcctattattgttatgcagatctcttcggaaacacctactggggccaggggacccaggtcaccgtctcctca

SEQ ID NO: 102 - DNA, 387 bp, Artificial Sequence
  Single-domain antibody nt sequence (Nb7)
caggtgcagctgcaggagtctggaggaggattggtgcaggctggggactctctgagactctcctgtctatcctctgaacgcacattcaggcgctatggcatagcctggttccgccaggctccagggaaggagcgtgagtttgtagcagctgttgaccggagtcatagtcagacaaactatgcagacttcgtacagggccgattcaccatctccacggtctacgccaagaacatggtgtatctgcaaatgaacagcctgaaacctgaggacacggccgtttattactgtgctgcggcgtcgtacgagaaagggtcggactatactagttggaacaccgacagagggtatgactactggggccaggggacccaggtcaccgtctcctca

SEQ ID NO: 103 - DNA, 384 bp, Artificial Sequence
  Single-domain antibody nt sequence (Nb43)
caggtgcagctgcaggagtctgggggaggattggtgcaggctggggactctctgaaactctcctgtgcagcctctggacgcaccttcagtgacagtgccatgggctggttccgccaggctccaggagaggagcgtgagtttgtagcagttattacctggaatggtggcaccacatactatgcagactccgtgaagggccgattcaccatctccagagacgacgccaagaacacggtgtacctgcaaatgaacagcctgaaacctgaggacacggccgtttattactgtgcagcggacacaagccggtgggactatagtcttacataccactacacgagggagtataactactggggccaggggacccaggtcaccgtctcctca

SEQ ID NO: 104 - DNA, 390 bp, Artificial Sequence
  Single-domain antibody nt sequence (Nb47)
caggtgcagctgcaggagtctgggggaggattggtgcaggctgggggctctctgagactctcctgtgcagcctctggacgcaccttgagtaggtattccatgagctggttccgccaggctccagggaaggagcgtgagtttgtagcaggtatacggtggagtggtagtaacacatactatgcagactccatgaagcagcgattcaccatctcccaagacaatgtcaagaacacggtgcatctgcaaatgaacagcctgaaacctgaggacacggccgtttattactgtgcagccacaagtagaagtgatcattacttgaatgccgtggcttggacccttccgaatgagtatgactactggggccaggggacccaggtcaccgtctcctca

SEQ ID NO: 105 - DNA, 360 bp, Artificial Sequence
  Single-domain antibody nt sequence (Nb50)
caggtgcagctgcaggagtctgggggaggattggtgcaggctgggggatctctgagactctcctgtgcagcctctggacgcaccttcgatggtacctatcgcatgggctggttccgccagggtccagggaaggagcgtgagtttgtagcagctataggcttcggtgttagtaccacatcgtatgcagactccgtgaagggccgattcaccatctccagaaacaacgccaagaacacggtgtatctgcaaatgaacagcctgaaacctgaggacacggccgtttattactgcgcagcgcgccccccgccttacacggagtataactactggggccaggggacccaggtcaccgtctcctca

SEQ ID NO: 106 - DNA, 372 bp, Artificial Sequence
  Single-domain antibody nt sequence (Nb53)
caggtgcagctgcaggagtctggaggaggagtggtgcaggctggggactctctgagactctcctgtgcagcctctggactcaccttcagtagttatctcatggcctggttccgccaggctccagggaaggagcgtgagtttgtagcacgtattaactggaatggtcgtgtgccatacactgtagactctgtgaagggccgattcatcatctccagagacaatgccaaaaacacggtgtggctgcaaatgaacagcctgaaacctgaggacacggccgtttattactgtgcagcagaccggaactacggcacagggggcgccgaaacagtgtatgagtactggggccaggggacccaggtcaccgtctcctca

SEQ ID NO: 107 - DNA, 375 bp, Artificial Sequence
  Single-domain antibody nt sequence (Nb55)
caggtgcagctgcaggagtctgggggaggattggtgcaggctgggggctctctgagactctcctgtgaagcctctggacgcgccttcagtgactcgtcggccatggcctggttccgccaggctccagggaaggagcgtgagtttgtagcggcgcttaacagagttaatgttgcatattgtagagactccgtgtcgggccgattcaccatctccagagacaacggcaagaatacggcatatctggaaatgaacagtgtgaaacctgaggacacggccatttattactgtgcatcagatctaatcctaaatgattgcagtcgaaaccccgcgaggtatgcctactggggccaggggacccaggtcaccgtctcctca

SEQ ID NO: 108 - DNA, 387 bp, Artificial Sequence
  Single-domain antibody nt sequence (Nb60)
caggtgcagctgcaggagtctgggggaggattggtgcaggctggggactctctgagactctcctgtctagcctctgaacgcacattcaggcgctatggcatgggctggttccgccaggctccagggaaggagcgtgagtttgtagcagctattgaccggagtcatagtaatacagactatgcagacttcgtgaagggccgattcaccatctccacggtctacgccaagaacatggtgtatctgcaaatgaacagcctgaaacctgaggacacggccgtttattactgtgctgcggcgacgtacgagaaagggtcggaccctactagttggaacaccgacagagggtatgacgtctggggccaggggacccaggtcaccgtctcctca

SEQ ID NO: 109 - DNA, 363 bp, Artificial Sequence
  Single-domain antibody nt sequence (msNb12)
caagtaaaacttgaagaatcaggtggtggaagcgttcaagctggtggatctctcagacttatttgtaccgcacccggcttaacacataataactgtggcttggactggtacagacgagcaccaggcaaggaacgcgagttcgtttcatcaataagtgcagatggaaccacttcatacgcagattccgtcaagggacggtttaccattagtaaggacaaagtcgaggacacagtctacctccaaatgaacagtttgaagccagaagacactgctatttattcatgcaagacagccttcccttacttcggtaatagctgtgttttggactactggggtcaaggaacctcagtcaccgtctcctcg

SEQ ID NO: 110 - DNA, 375 bp, Artificial Sequence
  Single-domain antibody nt sequence (mNb30)
caggtgcaattggtagagtctggcggtggactcgttcaagccggaggctcactgcggttgtcttgtgccgcatctggtctaaccttctcaaaatatgctatggggtggttccggcaggctccaggtaaagagcggaaatttgtcgcaacaattagttggtctggtgatagtgccttctatgctgattcagtaaaaggtcgattcactatatcacgggataacgcaagaaatactgtctatctccaaatgaactctctgaagcctgaagatactgctgtgtattactgtgccgcagatcgaggaatggggtatggggattttatggactactggggtcaaggaacctcagtcaccgcctcctcggcctcaggggcc

SEQ ID NO: 111 - DNA, 375 bp, Artificial Sequence
  Single-domain antibody nt sequence (mNb35)
caggtccaactggtagagtcaggcggtggactcgttcaagccggaggctcactgcggttgtcttgtgccgcatctggtcgaaccttctcaaaatatgctatggggtggttccggcaggctccaggtaaagagcggaaatttgtcgcaacaattagttggtctggtgatagtgccttctatgctgattcagtaaaaggtcgattcactatatcacgggataacgcaagaaatactgtctatctccaaatgaactctctgaagcctgaagatactgctgtgtattactgtaccgcagatcgaggaatggggtatggggattttatggactactggggtcaaggtacctcagtcaccgtctcctcggcctcaggggcc

SEQ ID NO: 112 - protein, 349 aa, Artificial Sequence
  Nb15-IgG1
QVQLQESGGGLVQAGGSLRVSCAASGLPFSDYLMGWFRQAPGKEREYVAAISQNGGHTYADSVLGRFTISRDNAKNTVYLQMNMLTPGDTAVYSCAARRPGGGRWDAAHDYNYWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 344 aa, Artificial Sequence
  Nb17-IgG1
QVQLQESGGGLVQTGGSLRLSCAASGRTFGIYRMGWFRQAPGKEREFVAAITSSADTAQYRDSVKGRFAISRDNAKNTLYLQMNSLKPEDTAIYYCAARDPTTLEYGNWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 344 aa, Artificial Sequence
  Nb19-IgG1
QVQLQESGGGLVQAGGSLRLSCAASGSGFSIHAMGWYRQAPGKQREFVAVVGHKTNYADSVKGRFTISRDVGKNTVELQMNSLKVEDTAVYYCYCNTIVTMTGVPDAVWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 353 aa, Artificial Sequence
  Nb56-IgG1
QVQLQESGGGLVQAGDSLRLSCVASERTFRRYGMGWFRQAPGKEREFVAAVDRSHTKTGYADFVKGRFTISTNYENMVYLQMNSLKPEDTAVYYCAAPSYEKGSDPTSWNTDRGYDYWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 371 aa, Artificial Sequence
  msNb12-IgG2a
QVKLEESGGGSVQAGGSLRLICTAPGLTHNNCGLDWYRRAPGKEREFVSSISADGTTSYADSVKGRFTISKDKVEDTVYLQMNSLKPEDTAIYSCKTAFPYFGNSCVLDYWGQGTSVTVSSEPKIPQPQPKPQPQPQPQPKPQPKPEPECTCPKCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 371 aa, Artificial Sequence
  mNb30-IgG2a
QVQLVESGGGLVQAGGSLRLSCAASGLTFSKYAMGWFRQAPGKERKFVATISWSGDSAFYADSVKGRFTISRDNARNTVYLQMNSLKPEDTAVYYCAADRGMGYGDFMDYWGQGTSVTASSEPKIPQPQPKPQPQPQPQPKPQPKPEPECTCPKCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 627 aa, Artificial Sequence
  Nb15-tri-IgG1
QVQLQESGGGLVQAGGSLRVSCAASGLPFSDYLMGWFRQAPGKEREYVAAISQNGGHTYADSVLGRFTISRDNAKNTVYLQMNMLTPGDTAVYSCAARRPGGGRWDAAHDYNYWGQGTQVTVSSGGGGSGGGGSGGGGSQVQLQESGGGLVQAGGSLRVSCAASGLPFSDYLMGWFRQAPGKEREYVAAISQNGGHTYADSVLGRFTISRDNAKNTVYLQMNMLTPGDTAVYSCAARRPGGGRWDAAHDYNYWGQGTQVTVSSGGGGSGGGGSGGGGSQVQLQESGGGLVQAGGSLRVSCAASGLPFSDYLMGWFRQAPGKEREYVAAISQNGGHTYADSVLGRFTISRDNAKNTVYLQMNMLTPGDTAVYSCAARRPGGGRWDAAHDYNYWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 612 aa, Artificial Sequence
  Nb17-tri-IgG1
QVQLQESGGGLVQTGGSLRLSCAASGRTFGIYRMGWFRQAPGKEREFVAAITSSADTAQYRDSVKGRFAISRDNAKNTLYLQMNSLKPEDTAIYYCAARDPTTLEYGNWGQGTQVTVSSGGGGSGGGGSGGGGSQVQLQESGGGLVQTGGSLRLSCAASGRTFGIYRMGWFRQAPGKEREFVAAITSSADTAQYRDSVKGRFAISRDNAKNTLYLQMNSLKPEDTAIYYCAARDPTTLEYGNWGQGTQVTVSSGGGGSGGGGSGGGGSQVQLQESGGGLVQTGGSLRLSCAASGRTFGIYRMGWFRQAPGKEREFVAAITSSADTAQYRDSVKGRFAISRDNAKNTLYLQMNSLKPEDTAIYYCAARDPTTLEYGNWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 612 aa, Artificial Sequence
  Nb19-tri-IgG1
QVQLQESGGGLVQAGGSLRLSCAASGSGFSIHAMGWYRQAPGKQREFVAVVGHKTNYADSVKGRFTISRDVGKNTVELQMNSLKVEDTAVYYCYCNTIVTMTGVPDAVWGQGTQVTVSSGGGGSGGGGSGGGGSQVQLQESGGGLVQAGGSLRLSCAASGSGFSIHAMGWYRQAPGKQREFVAVVGHKTNYADSVKGRFTISRDVGKNTVELQMNSLKVEDTAVYYCYCNTIVTMTGVPDAVWGQGTQVTVSSGGGGSGGGGSGGGGSQVQLQESGGGLVQAGGSLRLSCAASGSGFSIHAMGWYRQAPGKQREFVAVVGHKTNYADSVKGRFTISRDVGKNTVELQMNSLKVEDTAVYYCYCNTIVTMTGVPDAVWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 639 aa, Artificial Sequence
  Nb56-tri-IgG1
QVQLQESGGGLVQAGDSLRLSCVASERTFRRYGMGWFRQAPGKEREFVAAVDRSHTKTGYADFVKGRFTISTNYENMVYLQMNSLKPEDTAVYYCAAPSYEKGSDPTSWNTDRGYDYWGQGTQVTVSSGGGGSGGGGSGGGGSQVQLQESGGGLVQAGDSLRLSCVASERTFRRYGMGWFRQAPGKEREFVAAVDRSHTKTGYADFVKGRFTISTNYENMVYLQMNSLKPEDTAVYYCAAPSYEKGSDPTSWNTDRGYDYWGQGTQVTVSSGGGGSGGGGSGGGGSQVQLQESGGGLVQAGDSLRLSCVASERTFRRYGMGWFRQAPGKEREFVAAVDRSHTKTGYADFVKGRFTISTNYENMVYLQMNSLKPEDTAVYYCAAPSYEKGSDPTSWNTDRGYDYWGQGTQVTVSSDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 643 aa, Artificial Sequence
  msNb12-tri-IgG2a
QVKLEESGGGSVQAGGSLRLICTAPGLTHNNCGLDWYRRAPGKEREFVSSISADGTTSYADSVKGRFTISKDKVEDTVYLQMNSLKPEDTAIYSCKTAFPYFGNSCVLDYWGQGTSVTVSSGGGGSGGGGSGGGGSQVKLEESGGGSVQAGGSLRLICTAPGLTHNNCGLDWYRRAPGKEREFVSSISADGTTSYADSVKGRFTISKDKVEDTVYLQMNSLKPEDTAIYSCKTAFPYFGNSCVLDYWGQGTSVTVSSGGGGSGGGGSGGGGSQVKLEESGGGSVQAGGSLRLICTAPGLTHNNCGLDWYRRAPGKEREFVSSISADGTTSYADSVKGRFTISKDKVEDTVYLQMNSLKPEDTAIYSCKTAFPYFGNSCVLDYWGQGTSVTVSSEPKIPQPQPKPQPQPQPQPKPQPKPEPECTCPKCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 643 aa, Artificial Sequence
  mNb30-tri-IgG2a
QVQLVESGGGLVQAGGSLRLSCAASGLTFSKYAMGWFRQAPGKERKFVATISWSGDSAFYADSVKGRFTISRDNARNTVYLQMNSLKPEDTAVYYCAADRGMGYGDFMDYWGQGTSVTASSGGGGSGGGGSGGGGSQVQLVESGGGLVQAGGSLRLSCAASGLTFSKYAMGWFRQAPGKERKFVATISWSGDSAFYADSVKGRFTISRDNARNTVYLQMNSLKPEDTAVYYCAADRGMGYGDFMDYWGQGTSVTASSGGGGSGGGGSGGGGSQVQLVESGGGLVQAGGSLRLSCAASGLTFSKYAMGWFRQAPGKERKFVATISWSGDSAFYADSVKGRFTISRDNARNTVYLQMNSLKPEDTAVYYCAADRGMGYGDFMDYWGQGTSVTASSEPKIPQPQPKPQPQPQPQPKPQPKPEPECTCPKCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 194 aa, Artificial Sequence
  spike glycoprotein [Severe acute respiratory syndrome coronavirus 2]
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 125 - protein, 193 aa, Artificial Sequence
  spike glycoprotein S [SARS coronavirus BJ01]
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 126 - protein, 212 aa, Artificial Sequence
  spike glycoprotein [MERS coronavirus]
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 127 - protein, 223 aa, Artificial Sequence
  SARS CoV-2 spike receptor-binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 128 - protein, 1253 aa, Artificial Sequence
  spike glycoprotein [Severe acute respiratory syndrome coronavirus 2]
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLG

SEQ ID NO: 129 - protein, 243 aa, Artificial Sequence
  SARS-CoV-2 RBD polypeptide
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 130 - protein, 1256 aa, Artificial Sequence
  SARS-CoV-2 Spike polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH